(19) United States
(12) Patent Application Publication
(10) Pub. No.: US 2023/0059181 A1
(43) Pub. Date: Feb. 23, 2023

(54) IgE Antibody with Fcgamma Receptor binding

(71) Applicant: Epsilogen Ltd, London (GB)

(72) Inventors: Tim Wilson, London (GB); Kevin Fitzgerald, London (GB)

(21) Appl. No.: 17/764,850

(22) PCT Filed: Oct. 1, 2020
(86) PCT No.: PCT/EP2020/077608

§ 371 Date: Mar. 29, 2022

(30) Foreign Application Priority Data
Oct. 1, 2019 (GB) 1914165.4
Nov. 22, 2019 (GB) 1917059.6
Jun. 2, 2020 (GB) 2008248.3

(51) Int. Cl. C07K 16/28; C07K 16/30

(52) U.S. Cl. CPC C07K 16/283; C07K 16/30; C07K 2317/53; C07K 2317/524; C07K 2317/526; C07K 2317/732; C07K 2319/00

(57) ABSTRACT

Described herein are hybrid antibodies targeted for use in the treatment of cancer. The antibodies have binding capabilities for Fcε receptors and Fcγ receptors, which may be achieved e.g. by grafting heavy chain constant domain sequences (e.g. CH2 and CH3 domains) derived from IgG to IgE.

### FIELD OF THE INVENTION

The present invention lies in the design of synthetic (non-naturally occurring) hybrid antibodies, in particular hybrid IgE antibodies, together with their therapeutic use.

### BACKGROUND TO THE INVENTION

Immunoglobulin E (IgE) is a class of antibody (or immunoglobulin (Ig) “isotype”) that has only been found in mammals. IgE is synthesised by plasma cells. As with all antibody classes, monomers of IgE consist of two larger, identical heavy chains (ε chain) and two identical light chains (which are common to all antibody classes), with the ε chain containing four Ig-like constant domains (Cε1-Cε4: see FIG. 1).

It is the nature of the heavy chains that differentiates the different antibody classes, with those of the IgE class being larger and more heavily glycosylated than the heavy chains of the more common IgG class. Each antibody chain is comprised of a series of tandemly arranged immunoglobulin domains. The N-terminal domains (one each on the light and heavy chains) contain regions of highly variable sequence that enable binding to a huge range of antigens (the variable domains). The remaining domains consist of highly conserved so-called constant (Fc) domains.

IgE's main function is immunity to parasites such as helminths. IgE also has an essential role in type I hypersensitivity, which manifests in various allergic diseases, such as allergic asthma, most types of sinusitis, allergic rhinitis, food allergies, and specific types of chronic urticaria and atopic dermatitis. IgE also plays a pivotal role in responses to allergens, such as: anaphylactic drugs, bee stings, and antigen preparations used in desensitization immunotherapy.

Although IgE is typically the least abundant isotype, IgE levels in a normal (“non-atopic”) individual are only 0.05% of the Ig concentration, compared to 75% for the IgGs at 10 mg/ml, which are the isotypes responsible for most of the classical adaptive immune response and are capable of triggering the most powerful inflammatory reactions.

IgG is the main type of antibody found in blood and extracellular fluid, allowing it to control infection of body tissues. By binding many kinds of pathogens such as viruses, bacteria, and fungi, IgG protects the body from infection. IgG antibodies are large molecules with a molecular weight of about 150 kDa made of four peptide chains. Each molecule contains two identical class γ heavy chains of about 50 kDa and two identical light chains of about 25 kDa, thus a tetrameric quaternary structure. The two heavy chains are linked to each other and to a light chain each by disulphide bonds (see FIG. 1). The resulting tetramer has two identical halves which, together, form the Y-like shape. Each end of the fork contains an identical antigen binding site.

The structural differences confer different biological activities among the classes of antibody due to the panoply of effector cells and factors that bind to the different constant domains of each antibody class. The gamma chain of IgG binds to a family of receptors including FcγRI (CD64), FcγRIIa, FcγRIIb, FcγRIIIa (CD16) and FcγRIIIb. Similarly, the epsilon chain of IgE binds to a high affinity receptor, FcεRI and a lower affinity receptor FcεRII. The differential expression of these various receptors on differing immune effector cells determines the type of immune response that can be generated by IgG and IgE.

It is known that the receptor molecules that interact with IgG do so within the second constant domain of the gamma heavy chain (the CH2 domain). For example, the receptor on Natural Killer (NK) cells (FcγRIIIa) that interacts with IgG to enable recruitment and activation of these cells for cell/pathogen killing does so within the CH2 domain/lower hinge region. In contrast, it is the CH3 domain of IgE that is involved in the binding interaction with the IgE receptors (FcεRI and FcεRII) on its effector cells (mast cells, basophils, monocytes, macrophages, eosinophils; Scott C. et al (2012) Immunobiology 217: 1067-1079). IgE does not interact with FcγRIIIa and IgG does not interact with the FcεRI and FcεRII. Consequently, the two antibody classes mobilise distinct populations of effector cells and factors.

IgE is mostly known for its detrimental role in allergy, but several studies have long pointed towards a natural tumour surveillance function of this antibody isotype (Jensen-Jarolim E. et al (2008) Allergy 63: 1255-1266; Jensen-Jarolim E., Pawelec G. (2012) Cancer Immunol. Immunother. 61: 1355-1357). Pioneer studies with IgG and IgE antibodies of the same epitope specificity tested head-to-head revealed a higher potential of the IgE in terms of cytotoxicity (Gould H. J. et al (1999) Eur. J. Immunol. 29: 3527-3537).

IgE has evolved to kill tissue-dwelling multicellular parasites, endowing it with several key features that make it ideal for use in the treatment of solid tumours, which also mostly reside in tissue. The epsilon constant region of IgE has a uniquely high affinity for its cognate receptor (FcεRI) on the surfaces of immune effector cells including macrophages, monocytes, basophils and eosinophils (Ka˜10¹⁰/M for FcεRI and Ka˜10⁸-10⁹/M for the CD23 trimer complex; Gould H. J., Sutton B. J. (2008) Nat. Rev. Immunol. 8: 205-217). This interaction is up to 10,000-fold greater than the affinity that the gamma chain of IgG has for its cognate receptors and this results in the majority of IgE molecules being permanently attached to the surface of immune effector cells (Fridman W. H. (1991) FASEB J. 5: 2684-2690). Therefore, the latter are primed and ready to destroy cells expressing the antigen recognised by the IgE. As a result, IgE is able to permeate tissues more effectively than IgG and stimulate significantly greater levels of both antibody-dependent cell-mediated phagocytosis (ADCP) and antibody dependent cell-mediated cytotoxicity (ADCC), the two main mechanisms by which immune effector cells can kill tumour cells. Due to its rapid binding to Fcε-receptors on cells, IgE is quickly removed from the circulation and has a significantly longer tissue half-life than IgG (2 weeks versus 2-3 days), which is advantageous in terms of side-effects because of the short duration of the compound in the bloodstream and also supports a role in the killing of solid tumours.

Moreover, potential IgE-immunotherapies should be effectively distributed to tumour tissues because IgE antibodies bound to Fcε-receptors on e.g. mast cells can use those cells as shuttle systems to penetrate malignancies and, because mast cells are tissue-resident immune cells (St John A. L., Abraham S. N. (2013) J. Immunol. 190: 4458-4463), this transport would be highly efficient.

Other possible advantages include the high sensitivity of IgE-effector cells to activation by antigens and the speed and amplitude of the response, which can be seen most impressively during allergic and anaphylactic reactions, typically beginning within minutes upon allergen exposure. At the same time this is also the biggest concern of using IgE-based immunotherapies against cancer: recombinant IgE, applied intravenously, always bears the risk of anaphylactic reactions. Therefore, careful selection of the target epitope is of uttermost importance in this regard.

A challenge of current immunotherapies with IgG antibodies is that not all human Fcγ-receptors are immune-activating: one among them, FcγRIIb, is inhibiting (Nimmerjahn F., Ravetch J. V. (2006) Immunity 24: 19-28). Therefore, the tumouricidal effects of IgG-based immunotherapies also depend on the net ratio of binding to activating and inhibiting receptors. As has been shown for IgG4, a subclass that shows relatively high binding affinity to FcγRIIb (Bruhns P. et al (2009) Blood 113: 3716-3725), this antibody is not able to trigger immune cell-mediated tumour cell killing in vitro, despite being tumour associated antigen-specific. Moreover, it was demonstrated that IgG4 antibodies significantly impaired the killing potential of IgG1 antibodies of the same specificity in vitro and in vivo (Karagiannis P. et al (2013) J. Clin. Invest. 123: 1457-1474). Strategies to overcome this limitation include modification of the posttranslational glycosylation of the IgG-constant regions' heavy chains, as these sugar residues have been identified to be of high relevance for distinct binding affinities to different Fc-receptors (Schroeder H. W. Jr, Cavacini L. (2010) J. Allergy Clin. Immunol. 125: S41-52). For IgE on the other hand, there are no inhibitory receptors (Karagiannis S. N. et al (2012) Cancer Immunol. Immunother. 61: 1547-1564) so, again, this isotype could contribute to overcome a current challenge of immunotherapies of cancer.

Accordingly, there is a need for antibodies having improved properties compared to both IgE and IgG isotypes, and that are useful for example in the treatment of cancer.

### SUMMARY OF THE INVENTION

Despite the advantages of IgE over IgG in the solid tumour setting, IgG possesses certain functions that IgE lacks, such as activation of NK cells. Therefore, by exploiting the high degree of structural similarity among immunoglobulin domains, the present invention provides in one aspect IgE/IgG hybrid antibodies that possess the combined functionality of the IgG and IgE isotypes.

In one aspect, the present invention provides a hybrid antibody that binds Fcε receptors and Fcγ receptors. In this context, “binds” typically refers to binding of the hybrid antibody via one or more constant domains thereof, i.e. “binds” does not refer to specificity of the hybrid antibody binding to target antigen via its variable domains.

The term hybrid refers herein to an antibody whose structure is derived from more than one class of antibody. In the present invention, it is typically the Fc region that is a hybrid, thereby providing the antibody with the capability to bind to cell surface receptors of the immune system that are associated with different classes of antibody. Typically the hybrid antibody is capable of binding to and activating both an Fcε receptor and an Fcγ receptor, thereby transducing receptor signalling and effector functions in cells of immune system in which these receptors are expressed.

In one embodiment, the antibody of the present invention comprises one or more heavy chain constant domains derived from an IgE antibody (e.g. derived from an ε heavy chain). For instance, the antibody may comprise one or more domains selected from Cε1, Cε2, Cε3 and Cε4. Preferably the antibody comprises at least a Cε3 domain, more preferably at least Cε2, Cε3 and Cε4 domains.

In one embodiment, the hybrid antibody comprises a tetrameric IgE and at least one binding site for one or more Fcγ receptors. The one or more Fcγ receptor binding site(s) may be attached to the C-terminal of IgE. The tetrameric IgE may comprise a Fab region and an Fc region where the Fc domain comprises at least Cε2, Cε3 and Cε4 domains.

The fragment crystallisable/constant region (Fc region) is the tail region of an antibody that interacts with cell surface Fc receptors and some proteins of the complement system. This property allows antibodies to activate the immune system.

An Fcγ receptor binding site or sequence may be provided by way of one or more constant domains derived from IgG. Structural regions on IgE that exhibit homology to the regions on IgG where FcγR binds may be identified. Having identified such regions, amino acid substitutions may then be made to enable transfer of IgG functionality onto an IgE background.

Attachment of the one or more constant domains may be by any suitable attachment, link, graft, fixation or fusion. For example, the construct may include all or part of the hinge region derived from IgG. It will be appreciated that all or part of the constant domain sequence may be used, as well as variants thereof.

Thus in one embodiment, the hybrid antibody of the present invention comprises one or more heavy chain constant domains derived from an IgG antibody (e.g. derived from an γ heavy chain). For instance, the antibody may comprise one or more domains selected from Cγ1, Cγ2 and Cγ3. Preferably the antibody comprises at least a Cγ2 domain, more preferably at least Cγ2 and Cγ3 domains.

In one embodiment of the invention, the antibody has an Fc region comprising CH2, CH3 and CH4 domains derived from IgE (i.e. Cε2, Cε3 and Cε4 domains) and a CH2 domain, or variant thereof, derived from IgG (i.e. a Cγ2 domain). The antibody may further comprise the CH3 domain, or variant thereof, derived from IgG (i.e. a Cγ3 domain) and/or all or part of the hinge region derived from IgG.

In some embodiments, the antibody may comprise a wild type IgG hinge region, e.g. as shown in SEQ ID NO:9:

EPKSCDKTHTCPPCP (SEQ ID NO:9)

In some embodiments, the antibody may comprise a modified IgG hinge region. For instance, a potential free cysteine residue within the IgG hinge region may be replaced with another amino acid residue, e.g. to improve the stability of the hybrid antibody. In one embodiment, a cysteine residue present in the hinge region at position 220 of an IgG heavy chain sequence (numbering based upon the EU numbering scheme with reference to the IgG portion of the hybrid antibody) may be substituted for alternative amino acid residue (e.g. serine). Thus, the hybrid antibody may comprise e.g. a Cys220Ser amino acid substitution in the heavy chain IgG hinge region. Position 220 in the IgG heavy chain sequence referred to above corresponds to position 5 in SEQ ID NO:9, i.e. the hybrid antibody may comprise a variant of SEQ ID NO:9 lacking a C residue at position 5 (i.e. the antibody comprises a hinge region comprising a variant of SEQ ID NO:9 having a substitution at position 5).

Thus, in one embodiment, the antibody comprises a modified IgG hinge region as shown in SEQ ID NO:174:

EPKSSDKTHTCPPCP (SEQ ID NO:174)

The one or more IgG constant domains may include one or more amino acid substitutions or post-translational modifications to promote Fc receptor-mediated activity. For example, the CH2 domain may include glycosylation at position Asn297 thereof to assist with Fc receptor-mediated activity.

In a particular embodiment, the sequences, domains and regions derived from an IgG are derived from an IgG1 antibody. The antibody domains described herein may be derived from any species, preferably a mammalian species, more preferably from human.

In one embodiment, the hybrid antibody binds to FcγRIIIa. In another embodiment, the antibody binds to FcεRI. Preferably the hybrid antibody binds to both FcγRIIIa and FcεRI.

In some embodiments, the hybrid antibody is capable of binding to a neonatal Fc receptor (FcRn), typically in addition to a Fcγ receptor as described above. In alternative embodiments, the hybrid antibody is incapable of binding to FcRn, i.e. the antibody lacks FcRn-binding ability. For instance, the hybrid antibody may comprise one or more modified heavy chain constant domains derived from an IgG antibody, e.g. such that FcRn-binding of the modified antibody is reduced or eliminated (compared to a native IgG antibody). In one embodiment, the ability of the IgG portion of the hybrid antibody to bind to FcRn is removed by amino acid substitutions at specific residues known to be involved in FcRn binding. Such residues include Ile253, His310 and His435 in the IgG heavy chain sequence (numbering is based upon the EU numbering scheme with reference to the IgG portion of the hybrid antibody sequence). Thus, the hybrid antibody may comprise an IgG portion having one or more amino acid substitutions at positions 253, 310 or 435 in an IgG heavy chain sequence. For instance, the IgG portion of the hybrid antibody may comprise one or more of the following mutations: Ile253Ala, His310Ala and His435Ala. The sequence of a wild type IgG CH2 domain is shown in SEQ ID NO:10:

(SEQ ID NO: 10)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK

The sequence of a wild type IgG CH3 domain is shown in SEQ ID NO:11:

(SEQ ID NO: 11)
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

Positions 253, 310 or 435 in an IgG heavy chain sequence correspond to positions 23 and 80 in SEQ ID NO:10 and position 95 in SEQ ID NO:11 respectively. Thus, the hybrid antibody may comprise a variant of SEQ ID NO:10 (i.e. a modified IgG CH2 domain) comprising one or more amino acid substitutions at positions 23 and/or 80 (e.g. Ile23Ala and/or His80Ala). Alternatively, the hybrid antibody may comprise a variant of SEQ ID NO:11 (i.e. a modified IgG CH3 domain) comprising an amino acid substitution at position 95 (e.g. His95Ala). Preferably the hybrid antibody comprises a modified IgG CH2 domain and a modified IgG CH3 domain as described herein.

Thus in one embodiment, the hybrid antibody comprises a modified IgG CH2 domain and/or modified IgG CH3 domain (i.e. modified Cγ2 and/or Cγ3 domains) as shown in SEQ ID NO:175 and/or SEQ ID NO:176:

(SEQ ID NO: 175)
APELLGGPSVFLFPPKPKDTLMASRTPEVTCVVVDVSHEDPEVKFNWYVD
GVEVHNAKTKPREEQYNSTYRVVSVLTVLAQDWLNGKEYKCKVSNKALPA
PIEKTISKAK
(SEQ ID NO: 176)
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNAYTQKS
LSLSPGK

It will be appreciated that other receptor binding sites and desirable functions specific to IgG in the context of tumour targeting may also be grafted onto or into an IgE molecule to alter its functionality.

The hybrid antibody may further comprise a variable domain sequence that determines specific binding to one or more target antigen(s). Such variable domain sequences may be derived from any immunoglobulin isotype (e.g. IgA, IgD, IgE, IgG or IgM). In one embodiment, the variable domain sequence may be derived from IgE. In another embodiment, the variable domain sequence may be derived from IgG, e.g. IgG1. Alternatively, the variable domains may comprise sequences derived from two or more different isotypes, e.g. the variable domain may comprise a partial sequence derived from IgE and a partial sequence derived from IgG1. In one embodiment, the hybrid antibody comprises one or more complementarity-determining regions (CDRs) derived from an immunoglobulin isotype other than IgE (e.g. IgA, IgD, IgG or IgM, for example IgG1), and one or more framework regions and/or constant domains derived from an immunoglobulin of the isotype IgE.

The variable domains or portions thereof (e.g. the complementarity-determining regions (CDRs) or framework regions) may also be derived from the same or a different mammalian species to the constant domains present in the hybrid antibody. Thus, the hybrid antibody may be a chimaeric antibody, a humanized antibody or a human antibody.

Typically the variable domain(s) of the antibody binds to one or target antigens useful in the treatment of cancer, e.g. to a cancer antigen (i.e. an antigen expressed selectively on cancer cells or overexpressed on cancer cells) or to an antigen that inhibits or suppresses immune-mediated tumor cell killing. A sequence of one such variable domain sequence (i.e. of trastuzumab (Herceptin) IgE that binds to the cancer antigen HER2/neu) is shown in SEQ ID NO:1.

In some embodiments, the antibody may comprise an IgE amino acid sequence as defined in any one or more of SEQ ID NO:s 1 to 5, or a variant or fragment thereof. For instance, the hybrid antibody may comprise an amino acid sequence having at least 85%, 90%, 95% or 99% sequence identity with any one or more of the sequences of SEQ ID NOs:1 to 5. Preferably the antibody comprises at least SEQ ID NO:s 3, 4 and 5, or variants thereof, i.e. the antibody comprises amino acid sequences having at least 85%, 90%, 95% or 99% sequence identity with each of SEQ ID NO:3, SEQ ID NO:4 and SEQ ID NO:5.

In another embodiment, the hybrid antibody comprises an IgG CH2 amino acid sequence having at least 85%, 90%, 95% or 99% sequence identity with SEQ ID NO:10 or SEQ ID NO:175. In another embodiment, the antibody further comprises an IgG CH3 amino acid sequence having at least 85%, 90%, 95% or 99% sequence identity with SEQ ID NO:11 SEQ ID NO:176. In another embodiment, the antibody further comprises an IgG hinge amino acid sequence having at least 85%, 90%, 95% or 99% sequence with SEQ ID NO:9 or SEQ ID NO:174.

In a particular embodiment, the antibody comprises: i) an (e.g. IgE-derived) amino acid sequence having at least 85%, 90%, 95% or 99% sequence identity with any one or more of the sequences of SEQ ID NOs:1 to 5, preferably an amino acid sequence having at least 85%, 90%, 95% or 99% sequence identity with each of SEQ ID NO:3, SEQ ID NO:4 and SEQ ID NO:5 (more preferably at least SEQ ID NO:4); and ii) an (e.g. IgG1-derived) amino acid sequence having at least 85%, 90%, 95% or 99% sequence identity with SEQ ID NO:9, 10, 11, 174, 175 and/or 176 (more preferably at least SEQ ID NO:10 and SEQ ID NO:11 or at least SEQ ID NO:175 and SEQ ID NO:176).

The IgG-derived amino acid sequence is preferably attached to the C terminal of the IgE-derived amino acid sequence, either directly or using a suitable linker sequence. For instance, the sequence of SEQ ID NO:5 may be adjacent to the sequence of SEQ ID NO:9, 10, 11, 174, 175 or 176, preferably SEQ ID NO:9 or SEQ ID NO:174. Thus, in some embodiments, the hybrid antibody may comprise at least a Cε4 domain and at least an IgG hinge region and Cγ2 domains (including modified IgG hinge and/or Cγ2 domains), preferably at least a Cε4 domain and at least an IgG hinge region and Cγ2 and Cγ3 domains. Thus, the antibody may comprise an amino acid sequence having at least 85%, 90%, 95% or 99% sequence identity with SEQ ID NO:23 or SEQ ID NO:24.

In preferred embodiments, the antibody comprises a (e.g. heavy chain) amino acid sequence having at least 85%, 90%, 95% or 99% sequence identity with SEQ ID NO:25 or SEQ ID NO:26, most preferably SEQ ID NO:26, for example over at least 50, 100, 200, 300, 500 or 700 amino acid residues of, or over the full length of, SEQ ID NO:25 or SEQ ID NO:26.

In further embodiments, the antibody comprises a (e.g. heavy chain) amino acid sequence having at least 85%, 90%, 95% or 99% sequence identity with SEQ ID NO:163, 164, 165 or 166, most preferably SEQ ID NO:164 or 166, for example over at least 50, 100, 200, 300, 500 or 700 amino acid residues of, or over the full length of any of SEQ ID NOs: 163-166. In these embodiments, the antibody may optionally further comprise a light chain amino acid sequence having at least 85%, 90%, 95% or 99% sequence identity with SEQ ID NO:167, for example over at least 50, 100, 200, 300, 500 or 700 amino acid residues of, or over the full length of SEQ ID NO:167.

In further embodiments, the antibody comprises a (e.g. heavy chain) amino acid sequence having at least 85%, 90%, 95% or 99% sequence identity with SEQ ID NO:169, 170, 171 or 172, most preferably SEQ ID NO:170 or 172, for example over at least 50, 100, 200, 300, 500 or 700 amino acid residues of, or over the full length of any of SEQ ID NOs: 169-172. In these embodiments, the antibody may optionally further comprise a light chain amino acid sequence having at least 85%, 90%, 95% or 99% sequence identity with SEQ ID NO:173, for example over at least 50, 100, 200, 300, 500 or 700 amino acid residues of, or over the full length of SEQ ID NO:173.

Also described herein are antibodies comprising at least a CH3 domain or fragment thereof derived from IgE (i.e. a Cε3 domain) and one or more loop sequences from an IgG CH2 domain (i.e. a Cγ2 domain). Such antibodies may comprise a Cε3 domain in which one or more loop sequences (e.g. as defined in SEQ ID NO:s 6 to 8) are replaced by one or more FcγR-binding loops derived from a Cγ2 domain (e.g. as defined in SEQ ID NO:s 12 to 14). The loop sequences that are replaced in the Cε3 domain of IgE may show structural homology to the FcγR-binding loops in the Cγ2 domain of IgG. Such antibodies may comprise an amino acid sequence (e.g. encoding a hybrid Cε3/Cγ2 domain) having at least 85%, 90%, 95% or 99% sequence identity with any one or more of the sequences of SEQ ID NOs:15 to 22.

In another aspect the invention encompasses a hybrid antibody as defined hereinabove for use in treating or preventing cancer, e.g. benign or malignant tumours such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas.

Expressed in another way, the invention encompasses use of a hybrid antibody as described hereinabove in the manufacture of a medicament for administration to a human or animal for treating, preventing or delaying cancer, e.g. benign or malignant tumours such as melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas.

Expressed in a yet further way, the invention encompasses a method of preventing, treating and/or delaying cancer (e.g. benign or malignant tumours) in a mammal suffering therefrom, the method comprising administering to the mammal a therapeutically effective amount of the hybrid antibody as described hereinabove. It will be appreciated that the hybrid antibody of the invention may be administered in the form of a pharmaceutically acceptable composition or formulation.

In yet another aspect, the present invention resides in a composition comprising a hybrid antibody as described hereinabove and a pharmaceutically acceptable excipient, diluent or carrier. Optionally, the composition may further comprise a therapeutic agent such as another antibody or fragment thereof, aptamer or small molecule. The composition may be in sterile aqueous solution.

In a yet further aspect, there is provided a (recombinant) nucleic acid that encodes all or part of a heavy chain of a hybrid antibody, wherein the heavy chain comprises an amino acid sequence having at least 85%, 90%, 95% or 99% sequence identity with (i) one or more of SEQ ID NO:s 3 to 5 and (ii) SEQ ID NOs:10 and/or SEQ ID NO:11 or SEQ ID NO:175 and/or SEQ ID NO:176. In one embodiment, the nucleic acid encodes an amino acid sequence having at least 85%, 90%, 95% or 99% sequence identity with SEQ ID NO:23, SEQ ID NO:24, SEQ ID NO:25 or SEQ ID NO:26, preferably SEQ ID NO:24 or SEQ ID NO:26, or any one of SEQ ID NOs: 163-166 or SEQ ID NOs: 169-172.

There is also provided a vector comprising the nucleic acid as defined above, optionally wherein the vector is a CHO vector (i.e. an expression vector suitable for expression of the hybrid antibody in Chinese Hamster Ovary cells).

In a further aspect, there is provided a host cell comprising a recombinant nucleic acid encoding a hybrid antibody as described hereinabove or a vector as described herein, wherein the encoding nucleic acid is operably linked to a promoter suitable for expression in mammalian cells.

Also provided herein is a method of producing the hybrid antibody described hereinabove comprising culturing host cells as described herein under conditions for expression of the antibody and recovering the antibody or a fragment thereof from the host cell culture.

The hybrid antibodies described herein are highly stable, e.g. they typically show high thermal stability in denaturation studies. Preferably the hybrid antibodies are at least as thermally stable as a corresponding IgE antibody (e.g. an IgE antibody from which the hybrid antibody comprises one or more domains). More preferably the hybrid antibodies show improved stability compared to an IgE antibody.

### BRIEF DESCRIPTION OF THE DRAWINGS

FIG. 1: Schematic representation of IgE and IgG antibodies.

FIG. 2: A. Schematic of a hybrid antibody comprising IgG1 hinge, CH2 and CH3 domains (i.e. hinge, Cγ2 and Cγ3 domains) fused to a full IgE molecule via the C-terminal Cε4 domains thereof. B. SEC-HPLC chromatogram of the purified hybrid antibody. C. SDS-polyacrylamide gel electrophoresis of the purified hybrid antibody under non-denaturing or denaturing conditions, i.e. showing the size of the full antibody or single chains thereof against protein markers (in kDa).

FIG. 3: A schematic diagram of single cycle kinetic analysis of purified IgE-IgG Hinge-CH2-CH3 fusion protein binding to CD64 (FcγRI).

FIG. 4: Assay results showing binding of antibodies to CD64 (FcγRI). Binding of IgE-IgG Hinge-CH2-CH3 fusion protein to CD64 is similar to that of wild-type IgG1.

FIG. 5: Ribbon diagram illustrating the crystal structure of IgG1 Fc complexed with soluble FcγRIII (shown in green).

FIG. 6: Ball and stick image of overlay of IgE CH3 (top) and CH4 (bottom) in green and IgG CH2 (top) and CH3 (bottom) in blue.

FIG. 7: Schematic of domain engrafted IgE molecules prepared in accordance with an embodiment of the invention. Red domains are IgG CH domains, blue domains are IgE C domains, yellow domains are VH domains and green domains are the light chain V and C domains. A. IgG CH2 domains are fused to the C terminus of IgE. B. IgG CH2-CH3 domains are fused to the C terminus of IgE.

FIG. 8: A schematic diagram of an alternative single cycle kinetic analysis of purified hybrid antibodies binding to CD64 (FcγRI) or CD16A (FcγRIIIa).

FIG. 9: Assay results showing binding of hybrid antibodies to CD64 (FcγRI). Only hybrid antibodies comprising IgG CH2 or IgG CH2-CH3 domains are capable of binding CD64, although the off-rate for the fusion containing only IgG CH2 is faster than for the fusion containing IgG CH2-CH3.

FIG. 10: Assay results showing binding of hybrid antibodies to FcγRIIIA (CD16A). Only the hybrid antibody comprising IgG CH2-CH3 domains is capable of binding to CD16A.

FIG. 11: A schematic diagram of multiple cycle kinetic binding analysis of purified wild type IgE, Herceptin (trastuzumab IgG) and IgE-IgG hinge-CH2-CH3 fusion binding to FcεRIα.

FIG. 12: Assay results showing binding of hybrid antibodies to FcεRIα. Wild type IgE and the IgE-IgG hinge-CH2-CH3 fusion bind similarly to FcεRIα, whereas Herceptin does not bind to FcεRIα.

FIG. 13: Schematic of the vector expressing the IGEG.

FIG. 14: Schematic of the Biacore assay used to assess the binding of the Trastuzumab IGEG variants to human Her2 antigen by single cycle kinetic analysis.

FIG. 15: Human HER2: 1:1 binding of Trastuzumab IGEG variants.

FIG. 16: Schematic of the Biacore assay used to assess antibody binding to Fc gamma receptors.

FIG. 17: HMW-MAA IGEG (CH) variant binding to human Fc receptors. (a) Human FcgRI: 1:1 binding of CSP4 IGEG variants. (b) Human Fcε RIa: 1:1 binding of HMW-MAA IGEG variants. (c) Human FcγRIIIA_{176Val}: Binding of HMW-MAA IGEG variants—Raw Sensorgrams. (d) Human FcγRIIIA_{176Val}: Steady State binding of HMW-MAA IGEG variants—Analysed Data. In this figure, “CH” refers to anti-HMW-MAA (i.e. CSPG4), the variant designations are otherwise as described in Example 6.

FIG. 18: Schematic of the Biacore assay used to assess antibody binding to FcRn.

FIG. 19: HMW-MAA (CH) IGEG variant binding to human FcRn (a) FcRn pH 6.0: Binding of HMW-MAA IGEG variants—Raw Sensorgrams. (b) FcRn pH 6.0: Steady State binding of HMW-MAA IGEG variants—Analysed Data. (c) FcRn pH 7.4: Binding of HMW-MAA IGEG variants—Raw Sensorgrams (d) FcRn pH 7.4: Steady State binding of HMW-MAA IGEG variants—Analysed Data. In this figure, “CH” refers to anti-HMW-MAA (i.e. CSPG4), the variant designations are otherwise as described in Example 6.

FIG. 20: Biostability analysis of HMW-MAA (Hu CH) IGEG variants. (a) Fluorescence Thermal Melting Curves Overlay. (b) SLS 473 Stability Profile Curves Overlay. In this figure, “CH” refers to anti-HMW-MAA (i.e. CSPG4), the variant designations are otherwise as described in Example 6.

FIG. 21. Binding of anti-HMW-MAA (HuCH) IGEG Antibodies to A375 cells (a) Detection with anti-IgG secondary Antibody. (b) Detection with anti-IgE secondary Antibody. In this figure, “CH” refers to anti-HMW-MAA (i.e. CSPG4), the variant designations are otherwise as described in Example 6.

FIG. 22: R1, R2, R3 gating of data acquired from the Attune™ NxT Acoustic Focusing Cytometer.

FIG. 23: Effects of the Trastuzumab IgG, Herceptin IgG, Trastuzumab-IGEG (labelled CH2CH3), Trastuzumab-IGEG-C220S (labelled CH2CH3C220S) and Isotype IgG antibodies on antibody-dependent cell-mediated phagocytosis (ADCP) and antibody-dependent cell-mediated cytotoxicity (ADCC). (a) The effects of the antibodies on ADCP and ADCC at different concentrations (120-7.5 nM). (b) Graph showing the effects of the antibodies on ADCP and ADCC.

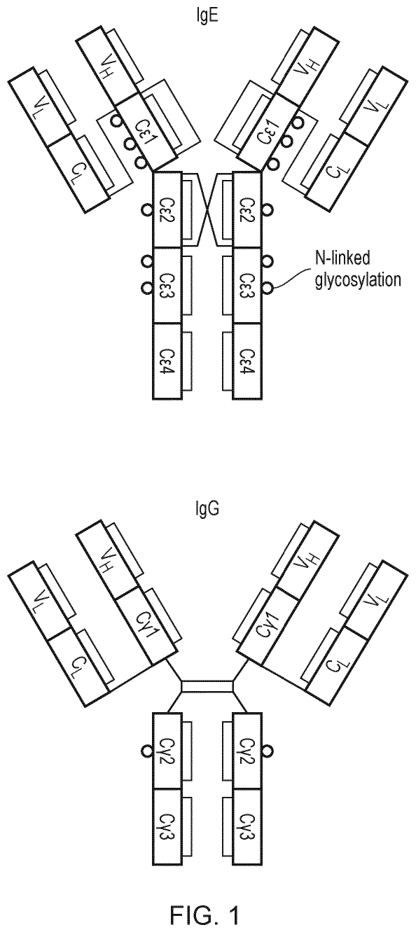

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein, the term “antibody” is used in its broadest sense and generally refers to an immunologic binding agent. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495) or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody may include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

Hence, also disclosed are methods for immunising animals, e.g., non-human animals such as laboratory or farm, animals using (i.e., using as the immunising antigen) any one or more (isolated) markers, peptides, polypeptides or proteins and fragments thereof as taught herein, optionally attached to a presenting carrier. Immunisation and preparation of antibody reagents from immune sera is well-known per se and described in documents referred to elsewhere in this specification. The animals to be immunised may include any animal species, preferably warm-blooded species, more preferably vertebrate species, including, e.g., birds, fish, and mammals. Without limitation, the antibodies may be chicken, turkey, goose, duck, guinea fowl, shark, quail or pheasant. Also without limitation, the antibodies may be human, murine (e.g., mouse, rat, etc.), donkey, rabbit, goat, sheep, guinea pig, shark, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

The invention described herein resides in IgE antibodies with an engineered heavy chain (Fc) portion resulting in hybrid IgE molecules. Structural regions on IgE were identified that exhibited homology to the regions on IgG where FcγRIIIa binds. Having identified such regions amino acid substitutions were made that enabled transfer of IgG functionality onto an IgE background. In particular, the IgG CH2 domain and the IgG CH2-CH3 region were fused to the C-terminus of IgE to impart gamma functionality onto IgE.

The hybrid antibodies described herein are typically capable of binding to Fcε receptors, e.g. to the FcεRI and/or the FcεRII receptors. Preferably the antibody is at least capable of binding to FcεRI (i.e. the high affinity Fcε receptor) or is at least capable of binding to FcεRII (CD23, the low affinity Fcε receptor).

Typically the antibodies are also capable of activating Fcε receptors, e.g. expressed on cells of the immune system, in order to initiate effector functions mediated by IgE. For instance, the antibodies may be capable of binding to FcγRI and activating mast cells, basophils, monocytes/macrophages and/or eosinophils.

The sites on IgE responsible for these receptor interactions have been mapped to peptide sequences on the Cc chain, and are distinct. The FcεRI site lies in a cleft created by residues between Gln 301 and Arg 376, and includes the junction between the Ca and Cε3 domains (Helm, B. et al. (1988) Nature 331, 180183). The FcεRII binding site is located within Cε3 around residue Val 370 (Vercelli, D. et al. (1989) Nature 338, 649-651). A major difference distinguishing the two receptors is that FcεRI binds monomeric Cc, whereas FcεRII will only bind dimerised Cc, i.e. the two Cc chains must be associated. Although IgE is glycosylated in vivo, this is not necessary for its binding to FcεRI and FcεRRII. Binding is in fact marginally stronger in the absence of glycosylation (Vercelli, D. et al. (1989) et. supra).

Thus binding to Fcε receptors and related effector functions are typically mediated by the heavy chain constant domains of the antibody, in particular by domains which together form the Fc region of the antibody. The antibodies described herein typically comprise at least a portion of an IgE antibody e.g. one or more constant domains derived from an IgE, preferably a human IgE. In particular embodiments, the antibodies comprise one or more domains (derived from IgE) selected from Cε1, Cε2, Cε3 and Cε4. In one embodiment, the antibody comprises at least Cε2 and Cε3, more preferably at least Cε2, Cε3 and Cε4, preferably wherein the domains are derived from a human IgE. In one embodiment, the antibody comprises an epsilon (c) heavy chain, preferably a human c heavy chain.

Constant domains derived from human IgE, in particular Cε1, Cε2, Cε3 and Cε4 domains, are shown in SEQ ID NO:s 2, 3, 4 and 5 respectively. Nucleic acid sequences encoding these acid sequences can be deduced by a skilled person according to the genetic code. The amino acid sequences of other human and mammalian IgEs and domains thereof, including human Cε1, Cε2, Cε3 and Cε4 domains and human c heavy chain sequences, are known in the art and are available from public-accessible databases. For instance, databases of human immunoglobulin sequences are accessible from the International ImMunoGeneTics Information System (IMGT®) website at http://www.imgt.org. As one example, the sequences of various human IgE heavy (c) chain alleles and their individual constant domains (Cε1-4) are accessible at http://www.imgt.org/IMGT GENE-DB/GENElect? query=2+IGHE& species=Homo+sapiens.

The hybrid antibodies described herein are typically capable of further binding to (e.g. human) Fcγ receptors, e.g. FcγRI (CD64), FcγRIIa, FcγRIIb, FcγRIIIa (CD16a) and/or FcγRIIIb (CD16b). In one embodiment the hybrid antibodies bind to FcγRI (CD64) and/or FcγRIIIa (CD16a). In another embodiment, the hybrid antibodies bind to FcγRI (CD64), FcγRIIIa (CD16a) and FcγRIIIb (CD16b). The hybrid antibodies may also bind to variants of FcγRIIIa (CD16a), e.g. human CD16a 176Phe and/or human CD16a 176Val. Preferably the antibody is at least capable of binding to FcγRI or is at least capable of binding to FcγRIIIa. More preferably the hybrid antibodies are capable of binding to and activating Fcγ receptors, and/or activating cells of the immune system expressing such receptors (including e.g. monocytes/macrophages and/or natural killer cells).

In some embodiments, the hybrid antibodies may further bind to the neonatal Fc receptor (FcRn). The hybrid antibody may bind to FcRn in a pH-dependent manner. For instance, the hybrid antibody may have a higher affinity for FcRn at pH 6.0 than at pH 7.4. The neonatal Fc receptor (FcRn) belongs to the extensive and functionally divergent family of MEW molecules. Contrary to classical MHC family members, FcRn possesses little diversity and is unable to present antigens. Instead, through its capacity to bind IgG and albumin with high affinity at low pH, it regulates the serum half-lives of both of these proteins. IgG enjoys a serum half-life that is substantially longer than similarly-sized globular proteins, including IgE which does not bind to FcRn (approximately 21 days for IgG and <2 days for IgE). In addition, FcRn plays important role in immunity at mucosal and systemic sites through both its ability to affect the lifespan of IgG as well as its participation in innate and adaptive immune responses.

FcRn has emerged as major modifier of monoclonal antibody (mAb) efficacy (Chan A. C., Carter P. J. (2010) Nat. Rev. Immunol. 10:301-16; Weiner L. M. et al (2010) Nat. Rev. Immunol. 10:317-27). This is directly related to the persistence of the therapeutic antibody in the bloodstream, which in turn can increase localisation to the target site. pH dependent binding and FcRn dependent recycling may be relevant to antibody function. Importantly, limited binding at neutral pH is required for proper release of IgG from cells and increasing the mAb affinity to FcRn at acidic pH correlates with half-life extension. Thus, IgG Fc engineering to optimise pH dependent binding to FcRn may be used in some cases to increase antibody half-life (see Dall'Acqua W. F. et al (2006) J. Biol. Chem. 281:23514-24; Yeung Y. A. et al (2009) J. Immunol. 182:7663-1; Zalevsky J. et al (2010) Nat. Biotechnol. 28:157-9).

However, in other embodiments, for instance where a shorter half-life of the antibody is desirable, it may be preferable to avoid FcRn binding. FcRn-binding ability may be conferred on the hybrid antibody by the presence of IgG heavy chain constant domains, e.g. IgG CH2 and CH3 domains as described above. In some embodiments, it may be desirable for the antibody to be capable of binding to Fcγ receptors such as FcγRI (CD64), FcγRIIIa (CD16a) and/or FcγRIIIb (CD16b), but to be incapable of binding FcRn. In such embodiments, the FcRn-binding ability of the antibody may be reduced or eliminated (compared to a native IgG antibody) by e.g. by amino acid substitutions at specific residues known to be involved in FcRn binding. Such residues include Ile253, His310 and His435 in the IgG heavy chain sequence (numbering is based upon the EU numbering scheme with reference to the IgG portion of the hybrid antibody sequence).

The antibodies described herein typically comprise at least a portion of an IgG antibody e.g. one or more constant domains derived from an IgG (e.g. an IgG1), preferably a human IgG. In particular embodiments, the antibodies comprise one or more domains (derived from IgG) selected from Cγ1, Cγ2 and Cγ3. In one embodiment, the antibody comprises at least Cγ2, more preferably at least Cγ2 and Cγ3, preferably wherein the domains are derived from a human IgG1 antibody. In one embodiment, the antibody further comprises a hinge region derived from IgG, e.g. IgG1.

Constant domains derived from human IgG, in particular Cγ2 and Cγ3 domains, are shown in SEQ ID NO:s 10 and 11 respectively. Nucleic acid sequences encoding these acid sequences can be deduced by a skilled person according to the genetic code. The amino acid sequences of other human and mammalian IgG constant domains, including human Cγ2 and Cγ3 domains and hinge sequences, are known in the art and are available from public-accessible databases, as described above for IgE constant domains.

The amino acid sequences of one or more IgE domain and one or more IgG domains may be linked directly or via a suitable linker. Suitable linkers for joining polypeptide domains are well known in the art, and may comprise e.g. 1, 2, 3, 4, 5, 6, 7, 8, 9 or 10 amino acid residues. In some embodiments, the linker sequence may comprise up to 20 amino acid residues.

Binding of the hybrid antibodies to FCC and Fcγ receptors may be assessed using standard techniques. Binding may be measured e.g. by determining the antigen/antibody dissociation rate, by a competition radioimmunoassay, by enzyme-linked immunosorbent assay (ELISA), or by Surface Plasmon Resonance (e.g. Biacore). Binding affinity may also be calculated using standard methods, e.g. based on the Scatchard method as described by Frankel et al., Mol. Immunol., 16:101-106, 1979.

In general, functional fragments of the sequences defined herein may be used in the present invention. Functional fragments may be of any length (e.g. at least 50, 100, 300 or 500 nucleotides, or at least 50, 100, 200, 300 or 500 amino acids), provided that the fragment retains the required activity when present in the antibody (e.g binding to an Fcγ and/or a Fcε receptors).

Variants of the amino acid and nucleotide sequences described herein may also be used in the present invention, provided that the resulting antibody binds both Fcγ and Fcε receptors. Typically such variants have a high degree of sequence identity with one of the sequences specified herein.

The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of the amino acid or nucleotide sequence will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of the specific antibody or a domain thereof described herein (e.g. a VL, VH, CL or CH domain) typically have at least about 75%, for example at least about 80%, 90%, 95%, 96%, 97%, 98% or 99% sequence identity with the original sequence (e.g. a sequence defined herein), for example counted over at least 20, 50, 100, 200 or 500 amino acid residues or over the full length alignment with the amino acid sequence of the antibody or domain thereof using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Typically variants may contain one or more conservative amino acid substitutions compared to the original amino acid or nucleic acid sequence. Conservative substitutions are those substitutions that do not substantially affect or decrease the affinity of an antibody to Fcγ and/or Fcε receptors. For example, a human antibody that binds the Fcγ and/or Fcε may include up to 1, up to 2, up to 5, up to 10, or up to 15 conservative substitutions compared to the original sequence (e.g. as defined above) and retain specific binding to the Fcγ and/or Fcε receptor. The term conservative variation also includes the use of a substituted amino acid in place of an unsubstituted parent amino acid, provided that the antibody binds Fcγ and/or Fcε. Non-conservative substitutions are those that reduce an activity or binding to Fcγ and/or Fcε receptors.

Functionally similar amino acids which may be exchanged by way of conservative substitution are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The domains described above (e.g. one or more IgE and IgG constant domains) are typically present in a heavy chain in the antibody. The hybrid antibody may further comprise one or more light chains in addition to one or more heavy chain sequences as described herein. Antibodies are typically composed of a heavy and a light chain, each of which has a variable region, termed the variable heavy (VH) region and the variable light (VL) region. Together, the VH region and the VL region are responsible for binding the antigen recognized by the antibody. Typically, a naturally occurring immunoglobulin has heavy (H) chains and light (L) chains interconnected by disulfide bonds. There are two types of light chain, lambda (λ) and kappa (k). Thus the hybrid antibodies typically comprise two heavy chains and two light chains (e.g. joined by disulfide bonds), e.g. based on an IgE antibody comprising an IgG hinge, CH2 and/or CH3 domain fused at the C-terminus of each heavy chain.

The hybrid antibodies described herein may bind specifically (i.e. via their variable domains or the complementarity determining regions (CDRs) thereof) to one or more target antigens useful in treating cancer. For instance, the hybrid antibodies may bind specifically to one or more cancer antigens (i.e. antigens expressed selectively or overexpressed on cancer cells). The novel combination of effector functions transduced via the combined FcεR- and FcγR-binding capability may enhance cytotoxicity, phagocytosis (e.g. ADCC and/or ADCP) and other cancer cell-killing function of immune system cells (e.g. monocytes/macrophages and natural killer cells). Preferably the hybrid antibodies are capable of inducing cytotoxicity (e.g. ADCC) and/or phagocytosis (ADCP), particularly against cancer cells. In a particularly preferred embodiment, the hybrid antibodies induce enhanced phagocytosis by immune cells (e.g. ADCP of cancer cells, by monocytes/macrophages or other effector cells, such as in an assay as described below in Example 8) compared to a corresponding IgE and/or IgG antibody. For example, the hybrid antibodies may bind specifically e.g. to EGF-R (epidermal growth factor receptor), VEGF (vascular endothelial growth factor) or erbB2 receptor (Her2/neu). One example of an antibody comprising variable domains that bind selectively to Her2/neu is trastuzumab (Herceptin).

In some embodiments, one or more of the variable domains and/or one or more of the CDRs, preferably at least three CDRs, or more preferably all six CDRs may be derived from one or more of the following antibodies: alemtuzumab (SEQ ID NOs:27-32), atezolizumab (SEQ ID NOs:33-38), avelumab (SEQ ID NOs:39-45), bevacizumab (SEQ ID NOs:46-51), blinatumomab, brentuximab, cemiplimab, certolizumab (SEQ ID NOs:52-57), cetuximab (SEQ ID NOs:58-63), denosumab, durvalumab (SEQ ID NOs:64-69), efalizumab (SEQ ID NOs:70-75), iplimumab, nivolumab, obinutuzumab, ofatumumab, omalizumab (SEQ ID NOs:76-81), panitumumab (SEQ ID NOs:82-87), pembrolizumab, pertuzumab (SEQ ID NOs:88-93), rituximab (SEQ ID NOs:94-99), or trastuzumab (SEQ ID NOs:100-105).

In such embodiments, the variable domains of the antibody may comprise one or more of the CDRs, preferably at least three CDRs, or more preferably all six of the CDR sequences from one of the antibodies listed in Table 1.

TABLE 1
Estimated CDR Amino Acid Sequences for Examples of Antibodies used in Cancer Therapy

| Antibody | CDR H1 | CDR H2 | CDR H3 | CDR L1 | CDR L2 | CDR L3 | Notes |
|---|---|---|---|---|---|---|---|
| Alemtuzumab | GFTF . . . TDFY | IRDKAKGYTT | AREGHT . . . AAP | QNIDKY | NTN | LQHIS . . . RPRT | 1 A |
| | (27) | (28) | FDY (29) | (30) | (31) | (32) | |
| Atezolizumab | DSWIH | WISPYGGSTY | RHWPGGF | DVST.AVA | SASFLY | QQYL.YHPAT | 2 B |
| | (33) | (34) | (35) | (36) | (37) | (38) | |
| Avelumab | SYIMM | SIYPSGGITF | .IKLFT . . . VTTV | VGGYNYVS | DVSNRP | SSYTSSSTRV | 2 B |
| | (39) | (40) | (41) | (42) | (43) | (45) | |
| Bevacizumab | GYTF...TNYG | INTY . . . TGEP | AKYPHYYGSS | QDISNY | FTS | QQYSTVPWT | 1 A |
| | (46) | (47) | HWYFDV (48) | (49) | (50) | (51) | |
| Certolizumab | GYVFT.DYGMN | GWL.NTYIGEPI | AR . . . G.YRSYAM | KASQNVGTN | SASFLY | QQYNIYPL | 3 A |
| | (52) | YADSVK.G (53) | DY (54) | VA (55) | (56) | (57) | |
| Cetuximab | GFSL . . . TNYG | IWSG . . . GNT | ARALTYY . . . DY | QSIGTN | YAS | QQNNN . . . WPTT | 1 A |
| | (58) | (59) | EFAY (60) | (61) | (62) | (63) | |
| Durvalumab | RYWMS | NIKQDGSEKY | EGGWFG . . . ELAF | RVSSSYLA | DASSRA | QQYG.SLPWT | 2 B |
| | (64) | (65) | (66) | (67) | (68) | (69) | |
| Efalizumab | GYSFT.GHWMN | GIMIHPSDSETR | ARIGIYFYGTT | RASKTISKYL | SGSTLQ | QQHNEYPL | 3 A |
| | (70) | YNQKFKDI (71) | YFDYI (72) | A (73) | (74) | (75) | |
| Omalizumab | GYSITSGYSWN | ASL . . . TYDGSTNY | ARGSHYF . . . GH | RASQSV.DYDGD | AASYLE | QQSHEDPY | 3 A |
| | (76) | ADSVK.G (77) | WHFAV (78) | SYMN (79) | (80) | (81) | |
| Panitumumab | GGSVS..SGDYY | IYYS . . . GNT | VRDRVTGA | QDISNY | DAS | QHFDH . . . LPLA | 1 A |
| | (82) | (83) | FDI (84) | (85) | (86) | (87) | |
| Pertuzumab | GFTF . . . TDYT | VNPN . . . SGGS | ARNLGP . . . SFY | QDVSIG | SAS | QQYYI . . . YPYT | 1 A |
| | (88) | (89) | FDY (90) | (91) | (92) | (93) | |
| Rituximab | GYTF . . . TSYN | IYPG . . . NGDT | ARSTYYG..GD | SSVSY | ATS | QQWTS . . . NPPT | 1 A |
| | (94) | (95) | WFNV (96) | (97) | (98) | (99) | |
| Trastuzumab | GFNI . . . KDTY | IYPT . . . NGYT | SRWGGDG . . . FY | QDVNTA | SAS | QQHYT . . . TPPT | 1 A |
| | (100) | (101) | AMDY (102) | (103) | (104) | (105) | |

Numbers indicated in brackets are the corresponding SEQ ID NOs. Dots indicate sequence alignment gaps according to the IMGT and Kabata numbering systems. Letters indicate the method used to predict the CDR sequence. A—IMGT, B—Kabat. 1—Magdelaine-Beuzelin et al. (2007) Structure-function relationships of the variable domains of monoclonal antibodies approved for cancer treatment. Critical Reviews in Oncology/Hematology, 64: 210-225. 2 . . . Lee et al. (2017). Molecular mechanism of PD-l/PD-Ll blockade via anti-D-L1 antibodies atezolizumab and durvalumab. Scientific Reports, 7: 5532.3—Ling et al. (2018) Effect ofVH-VL Families in Pertuzumab and Trastuzumab Recombinant Production, Her2 and FcγllA Binding. Frontiers in Immunology, 9: 469.

In alternative embodiments, one or more of the variable domains and/or one or more CDRs, preferably at least three CDRs, or more preferably all six CDRs, may be derived from one or more of the following antibodies: abciximab, adalimumab (SEQ ID NOs:106-111), aducanumab, aducanumab, alefacept, alirocumab, anifrolumab, balstilimab, basiliximab (SEQ ID NOs:112-117), belimumab (SEQ ID NOs:118-123), benralizumab, bezlotoxumab, brodalumab, brolucizumab, burosumab, cankinumab, caplacizumab, crizanlizumab, daclizumab (SEQ ID NOs:124-129), daratumumab, dinutuximab, dostarlimab, duplilumab, eclizumab, elotuzumab, emapalumab, emicizumab, epitinezumab, erenumab, etrolizumab, evinacumab, evolocumab, fremanezumab, galcanezumab, golimumab, guselkumab, ibalizumab, idarucizumab, inebilizumab, infliximab (SEQ ID NOs:130-135), isatuximab, ixekizumab, lanadelumab, leronlimab, margetuximab, mepolizumab, mogamulizumab, muromonab, narsoplimab, natalizumab (SEQ ID NOs:136-141), naxitamab, necitumumab, obiltoxaximab, ocrelizumab, omburtamab, palivizumab (SEQ ID NOs:142-147), ramucirumab, ranibizumab (SEQ ID NOs:148-153), reslizumab, risankizumab, romosozumab, sarilumab, satralizumab, secukinumab, spartalizumab, sutimlimab, tafasitamab, tanezumab, teplizumab, teprotumumab, tildrakizumab, toclizumab, toropalimab, ustekinumab, vedolizumab or zalifrelimab.

In such embodiments, the variable domains of the antibody may comprise one or more of the CDRs, preferably at least three CDRs, or more preferably all six of the CDR sequences from one of the antibodies listed in Table 2.

TABLE 2
Estimated CDR Amino Acid Sequences for Example Therapeutic Antibodies

| Antibody | CDR HI | CDRH2 | CDRH3 | CDR Li | CDR L2 | CDR L3 | Notes |
|---|---|---|---|---|---|---|---|
| Adalimumab | DYAMH | AITWNSGHIDYADSVEG | VSYLSTASSLDY | RASQGIRNYLA | AASTLQS | QRYNRAPYT | 1 A |
| | (106) | (107) | (108) | (109) | (HO) | (111) | |
| Basiliximab | GYSFTR . . . | AIYPGNSD . . . | DYGYYFDF | SASSSRSYMQ | DTSKLAS | HQRSS..YT | 2 |
| | YWMH | TSYNQKFEG | (H4) | (H5) | (H6) | (H7) | |
| | (112) | (H3) | | | | | |
| Belimumab | GGTFNNNAIN | GIIPMFGTAKYSQNFQG | SRDLLLFPHHALSP | QGDSLRSYYAS | GKNNRPS | SSRDSSGNHWV | 3B |
| | (118) | (119) | (120) | (121) | (122) | (123) | |
| Daclizumab | GYTFTS . . . | YINPSTGY . . . | GGGVFDY | SASSSISYMH | TTSNLAS | HQRSTYPLT | 2 |
| | YRMH | TEYNQKFKD | (126) | (127) | (128) | (129) | |
| | (124) | (125) | | | | | |
| Infliximab | IFSNHW | RSKSINSATH | N . . . YYGSTY | FVGSSIH | KYASESM | QSHSW | 4 |
| | (130) | (131) | (132) | (133) | (134) | (135) | |
| Natalizumab | GFNIKD . . . | RIDPANGY . . . | EGYYGNYGVYAMDY | KTSQDINKYMA | YTSALQP | LQYDN.LWT | 2 |
| | TYIH | TKYDPKFQG | (138) | (139) | (140) | (141) | |
| | (136) | (137) | | | | | |
| Palivizumab | GFSLSTSGMSVG | DIWWDDK . . . | SM . . . ITNWYFDV | KCQLSVGYMH | DTSKLAS | FQGSGYPFT | 2 |
| | (142) | KDYNPSLKS | (144) | (145) | (146) | (147) | |
| | | (143) | | | | | |
| Ranibizumab | GYDFTH . . . | WINTYTGE . . . | YPYYYGTSHWFDV | SASQDISNYLN | FTSSLHS | QQYSTVPWT | 2 |
| | YGMN | PTYAADFKR | (150) | (151) | (152) | (153) | |
| | (148) | (149) | | | | | |

Numbers indicated in brackets are the corresponding SEQ ID NOs. Dots indicate sequence alignment gaps according to the IMGT and Kabata numbering systems. Letters indicate the method used to predict the CDR sequence. AIMGT, BTreatment of Rheumatoid Arthritis. International Journal of Molecular Sciences, 19(3): pii E768.Kabat. 1—Schroter et al. (2014) A generic approach to engineer antibody pH-switches using combinatorial histidine scanning libraries and yeast display. MAbs, 7(1): 138-151. 2—Wang et al. (2009). Potential aggregation prone regions in biotherapeutics. A survey of commercial monoclonal antibodies. MAbs, 1(3): 254-267. 3—WO 2015/173782 AL 4—Lim et al. (2018). Structural Biology of the TNFa Antagonists Used in the Treatment of Rheumatoid Arthritis. International Journal of Molecular Sciences, 19(3): pii E768.

In other embodiments, one or more of the variable domains and/or one or more of the CDR sequences, preferably at least three CDRs, or more preferably all six CDRs, may be derived from an anti-HMW-MAA antibody. In one embodiment, one or more of the variable domains and/or one or more of the CDR sequences, preferably at least three CDRs, or more preferably all six CDRs may be derived from the anti-HMW-MAA antibody described in WO 2013/050725 (SEQ ID NOs:161 and 162 for the variable domain and SEQ ID NOs:154-159 for CDR). HMW-MAA refers to high molecular weight-melanoma associated antigen, also known as chondroitin sulfate proteoglycan 4 (CSPG4) or melanoma chondroitin sulfate proteoglycan (MCSP)—see e.g. Uniprot Q6UVK1.

In such embodiments, the variable domains of the antibody may comprise one or more of the CDR sequences, preferably at least three CDRs, or more preferably all six of the CDR sequences defined in Table 3. In other embodiments, one or more of the variable domains of the antibody comprises one or more of the variable domain sequences listed in Table 3.

TABLE 3
Estimated Variable Domains and CDR Sequences
of an Anti-HMW-MAA Antibody

| | SEQ | |
|---|---|---|
| Region | ID NO. | Amino Acid Sequence |
| CDR H1 | 154 | GFTFSNYW |
| CDR H2 | 155 | IRLKSNNFGR |
| CDR H3 | 156 | TSYGNYVGHYFDH |
| CDR L1 | 157 | QNVDTN |
| CDR L2 | 158 | SAS |
| CDR L3 | 159 | QQYNSYPLT |
| Variable Domain | 161 | EQVKLQQSGGGLVQPGGSMKLSCVVSGFTFSNYWM |
| (Heavy Chain) | | NWVRQSPEKGLEWIAEIRLKSNNFGRYYAESVKGRF |
| | | TISRDDSKSSAYLQMINLRAEDTGIYYCTSYGNYVGH |
| | | YFDHWGQGTTVTVSS |
| Alternative | 177 | EVQLVQSGGGLVQPGGSLKLSCAVSGFTFSNYWMN |
| Variable Domain | | WVRQAPGKGLEWVGEIRLKSNNFGRYYAESVKGRF |
| (Heavy Chain) | | TISRDDSKNTAYLQMNSLKTEDTAVYYCTSYGNYVG |
| | | HYFDHWGQGTLVTVSS |
| Variable Domain | 162 | DIELTQSPKFMSTSVCDRVSVTCKASQNVDTNVAWY |
| (Light Chain) | | QQKPGQSPEPLLFSASYRYTGVPDRFTGSGSGTDFTL |
| | | TISNVQSEDLAEYFCQQYNSYPLTFGGGTKLEIK |
| Alternative | 178 | DIQLTQSPSFLSASVGDRVTITCKASQNVDTNVAWYQ |
| Variable Domain | | QKPGKAPKPLLFSASYRYTGVPSRFSGSGSGTDFTLTI |
| (Light Chain) | | SSLQPEDFATYFCQQYNSYPLTFGGGTKVEIK |

In some embodiments, the hybrid antibody binds to a target antigen with a dissociation constant (Kd) of less than 1 μM, preferably less than 1 nM. For instance, in one embodiment the hybrid antibody binds to human Her2 or HMW-MAA with a Kd of 1×10⁻⁹ (1 nM) or lower.

Compositions are provided herein that include a carrier and one or more hybrid antibodies that bind Fcγ and Fcε receptors, or functional fragments thereof. The compositions can be prepared in unit dosage forms for administration to a subject. The amount and timing of administration are at the discretion of the treating physician to achieve the desired purposes. The antibody can be formulated for systemic or local (such as intra-tumour) administration. In one example, the antibody is formulated for parenteral administration, such as intravenous administration.

The compositions for administration can include a solution of the antibody or a functional fragment thereof) dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical dose of the pharmaceutical composition for intravenous administration includes about 0.1 to 15 mg of antibody per kg body weight of the subject per day. Dosages from 0.1 up to about 100 mg per kg per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Antibodies may be provided in lyophilized form and rehydrated with sterile water before administration, although they are also provided in sterile solutions of known concentration. The antibody solution is then added to an infusion bag containing 0.9% sodium chloride, USP, and typically administered at a dosage of from 0.5 to 15 mg/kg of body weight. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The antibody described herein (or functional fragment thereof) can be administered to slow or inhibit the growth of cells, such as cancer cells. In these applications, a therapeutically effective amount of an antibody is administered to a subject in an amount sufficient to inhibit growth, replication or metastasis of cancer cells, or to inhibit a sign or a symptom of the cancer. In some embodiments, the antibodies are administered to a subject to inhibit or prevent the development of metastasis, or to decrease the size or number of metasases, such as micrometastases, for example micrometastases to the regional lymph nodes (Goto et al., Clin. Cancer Res. 14(11):3401-3407, 2008).

A therapeutically effective amount of the antibody will depend upon the severity of the disease and the general state of the patient's health. A therapeutically effective amount of the antibody is that which provides either subjective relief of a symptom(s) or an objectively identifiable improvement as noted by the clinician or other qualified observer. These compositions can be administered in conjunction with another chemotherapeutic agent, either simultaneously or sequentially.

Many chemotherapeutic agents are presently known in the art. In one embodiment, the chemotherapeutic agents is selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti-angiogenesis agents.

All documents cited in the present specification are hereby incorporated by reference in their entirety. The invention will now be described in more detail by way of the following non-limiting examples.

### EXAMPLES

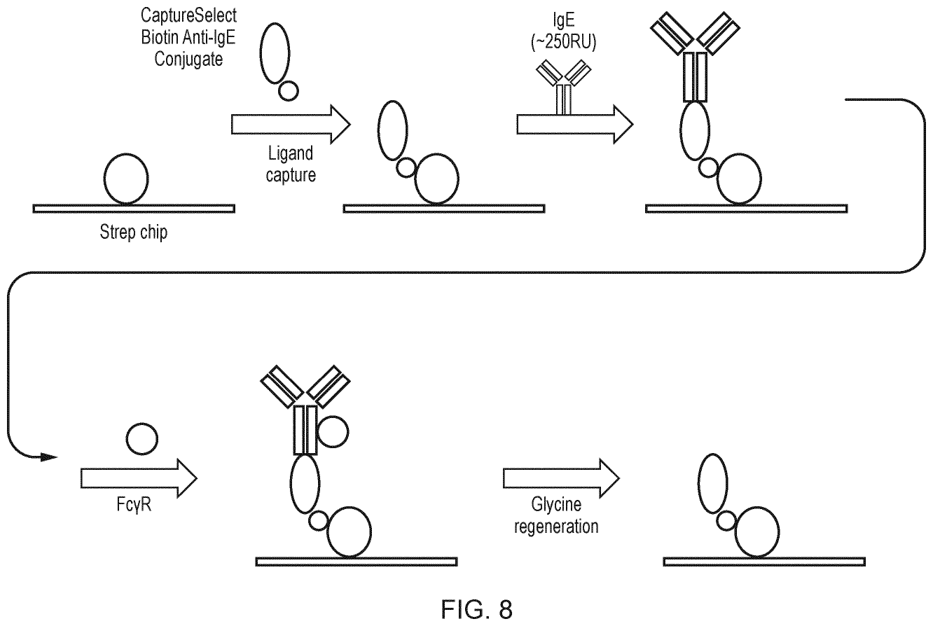

Antibody dependent cellular cytotoxicity (ADCC) as mediated by IgG occurs when antibody that is bound to a target pathogen or cell is able to coincidently bind to FcγRIIIa on natural killer cells (NK cells). The NK cells so recruited release a cocktail of factors (e.g. granzymes, perforin) that result in destruction of the antibody opsonised pathogen/targeted cell. FcγRIIIa binds to a region of IgG in the CH2 domain that is proximal to the hinge region (see FIG. 8; Sondermann et al (2000) Nature 406: 267-73).

The FcγRIIIa binding region of IgG has been compared with regions of the CH3 and CH4 domains of IgE to identify regions of structural homology. As can be seen in FIG. 6, the CH2 and CH3 domains of IgG occupy very similar 3D space to the CH3 and CH4 domains of IgE.

A combination of amino acid sequence alignment, secondary structure prediction and inspection of the structures for IgG and IgE shown in FIG. 6 resulted in the design of a number of variant IgE molecules which have been constructed to incorporate mutations aimed at substituting regions of the IgG CH2 domain into homologous regions of the IgE backbone to accommodate FcγRIIIa binding. These variants were expressed and receptor binding assays and tumour cell killing assays performed (both by NK cells as well as IgE's normal effector cells).

It is known that glycosylation at position Asn297 of the CH2 domain of IgG is also required for FcγRIIIa binding and activation of NK cells. This indicates that a potentially complex conformational epitope may be necessary for FcγRIIIa binding. The very different glycosylation of IgE might therefore make the projection of a FcγRIIIa binding site on IgE difficult to achieve through inspection of amino acid sequence alignment and structural homology modelling.

In the following examples, it is demonstrated that FcγR-binding (e.g. FcγRIIIa binding) can be conferred on an IgE antibody by fusing at least the IgG hinge, CH2 and CH3 domains onto the C-terminus of the heavy chain of the antibody.

### Example 1—FcγR Binding Site Engraftment

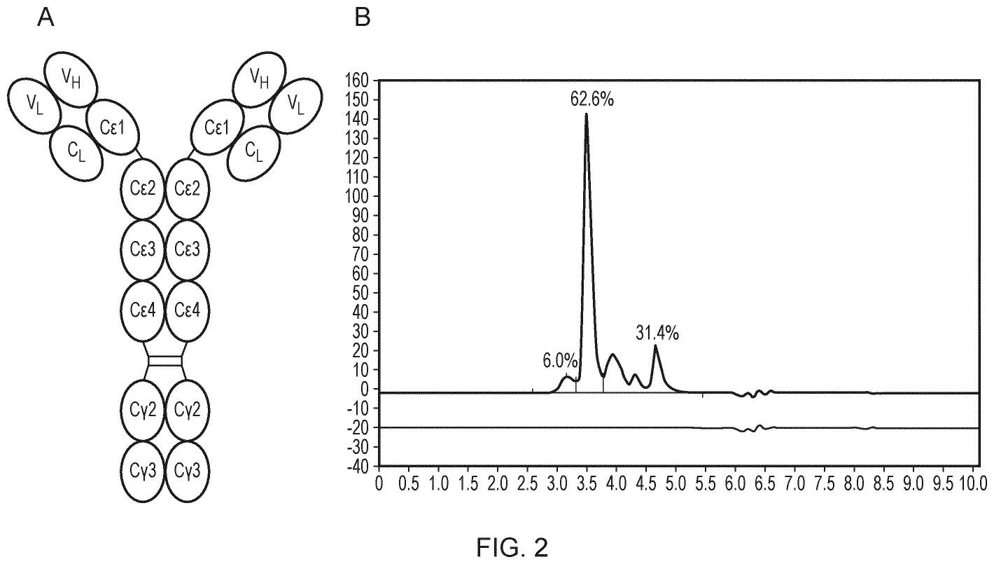

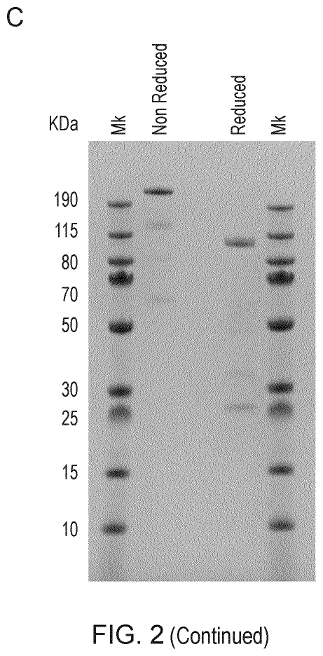

In this example, an IgE variant was created in which the IgG hinge and IgG CH2-CH3 domain pair was fused to the IgE framework at the C terminus (FIG. 2A). The IgE antibody is based on trastuzumab IgE, e.g. as disclosed in Karagiannis et al., Cancer Immunol Immunother. (2009) June; 58(6):915-30.

Another IgE variant was created in which the IgG hinge and CH2 domain was fused to the C terminus of trastuzumab IgE.

Further variant IgE antibodies were generated in which one or more loops in a Cε3 domain of the IgE were replaced by one or more FcγR-binding loops derived from a Cγ2 domain of an IgG antibody. The loops that are replaced in the Cε3 domain of the IgE show structural homology to the FcγR-binding loops in the Cγ2 domain of IgG.

Methods

Cloning:

DNA sequences corresponding to both the wild type (WT) trastuzumab IgE constant domain and separately, IgE containing IgG FcγR-binding Loop 1+Loop 2+Loop 3 were synthesised with flanking restriction enzyme sites for cloning into Abzena's pANT dual Ig expression vector system for human heavy and kappa light chains. The heavy chains, also containing Trastuzumab VH, were cloned between the Mlu I and KpnI restriction sites. Trastuzumab Vk, synthesised separately, was cloned between the Pte I and BamH I restriction sites. Individual loop variants were constructed using specific primers to amplify the loop(s) of interest and pulled through PCR to generate IgE with either one or two IgG1 loops in all possible combinations to generate a total of six additional constructs (1, 2, 3, 1+2, 1+3, 2+3).

To generate IgE-IgG1-CH2 and CH2-CH3 fusion variants, specific primers were used to amplify WT IgE whilst removing the stop codon at the end of IgE CH4 and, in a separate reaction, to amplify either IgG1 CH2 or IgG1 CH2-CH3 which were synthesised separately. Pull through PCR was used to combine both fragments and introduce Mlu I and KpnI restriction sites for cloning into the dual expression vector. FIG. 7 is a stylised diagram of the two fusion variants with FIG. 7a illustrating IgE-IgG1-CH2 and FIG. 7b illustrating IgE-IgG1-CH2-CH3.

Sequences:

The following hybrid antibody molecules have been constructed:
IgE containing IgG FcγR Loop 1;
IgE containing IgG FcγR Loop 2;
IgE containing IgG FcγR Loop 3;
IgE containing IgG FcγR Loop 1+Loop 2;
IgE containing IgG FcγR Loop 1+Loop 3;
IgE containing IgG FcγR Loop 2+Loop 3; and
IgE containing IgG FcγR Loop 1+Loop 2+Loop 3.

In addition, the following fusion proteins have been constructed:
IgE plus IgG1 Hinge-CH2
IgE plus IgG1 Hinge-CH2-CH3

The sequences for wild type Trastuzumab IgE were as follows:

WT IgE_VH:
(SEQ ID NO: 1)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS
WT IgE_VL:
(SEQ ID NO: 160)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
WT IgE_CH1:
(SEQ ID NO: 2)
ASTQSPSVFPLTRCCKNIPSNATSVTLGCLATGYFPEPVMVTWDTGSLNG
TTMTLPATTLTLSGHYATISLLTVSGAWAKQMFTCRVAHTPSSTDWVDNK
TFS
WT IgE_CH2:
(SEQ ID NO: 3)
VCSRDFTPPTVKILQSSCDGGGHFPPTIQLLCLVSGYTPGTINITWLEDG
QVMDVDLSTASTTQEGELASTQSELTLSQKHWLSDRTYTCQVTYQGHTFE
DSTKKCA
WT IgE_CH3 (loops changed are underlined):
(SEQ ID NO: 4)
DSNPRGVSAYLSRPSPFDLFIRKSPTITCLVVDLAPSKGTVNLTWSRASG
KPVNHSTRKEEKQRNGTLTVTSTLPVGTRDWIEGETYQCRVTHPHLPRAL
MRSTTKTS
WT IgE_CH4:
(SEQ ID NO: 5)
GPRAAPEVYAFATPEWPGSRDKRTLACLIQNFMPEDISVQWLHNEVQLPD
ARHSTTQPRKTKGSGFFVFSRLEVTRAEWEQKDEFICRAVHEAASPSQTV
QRAVSVNPGK

IgE Loop 1: LAPSKGT (SEQ ID NO:6);

IgE Loop 2: RNGTLT (SEQ ID NO:7)

IgE Loop 3: HPHLPRA (SEQ ID NO:8)

Sequences for wild type IgG were as follows:

WT IgG_Hinge:
(SEQ ID NO: 9)
EPKSCDKTHTCPPCP
WT IgG_CH2 (loops changed italicised and
underlined):
(SEQ ID NO: 10)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK
WT IgG_CH3:
(SEQ ID NO: 11)
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

IgG FcγR-binding Loop 1: VSHEDPE (SEQ ID NO:12)

IgG FcγR-binding Loop 2: YNSTYR (SEQ ID NO:13) IgG FcγR-binding Loop 3: NKALPAP (SEQ ID NO:14)

Sequences for the hybrid molecules were as follows. Each hybrid molecule further comprises wild-type IgE_VH, IgE_CH1, IgE_CH2 and IgE_CH4 (i.e. SEQ ID NOs:1, 2, 3 and 5)

IgE_CH3 containing IgG FcγR-binding Loop 1:
(SEQ ID NO: 15)
DSNPRGVSAYLSRPSPFDLFIRKSPTITCLVVDVSHEDPEVNLTWSRASG
KPVNHSTRKEEKQRNGTLTVTSTLPVGTRDWIEGETYQCRVTHPHLPRAL
MRSTTKTS
IgE_CH3 containing IgG FcγR-binding Loop 2:
(SEQ ID NO: 16)
DSNPRGVSAYLSRPSPFDLFIRKSPTITCLVVDLAPSKGTVNLTWSRASG
KPVNHSTRKEEKQYNSTYRVTSTLPVGTRDWIEGETYQCRVTHPHLPRAL
MRSTTKTS
IgE_CH3 containing IgG FcγR-binding Loop 3:
(SEQ ID NO: 17)
DSNPRGVSAYLSRPSPFDLFIRKSPTITCLVVDLAPSKGTVNLTWSRASG
KPVNHSTRKEEKQRNGTLTVTSTLPVGTRDWIEGETYQCRVTNKALPAPL
MRSTTKTS
IgE_CH3 containing IgG FcγR-binding Loop 1 +
Loop 2:
(SEQ ID NO: 18)
DSNPRGVSAYLSRPSPFDLFIRKSPTITCLVVDVSHEDPEVNLTWSRASG
KPVNHSTRKEEKQYNSTYRVTSTLPVGTRDWIEGETYQCRVTHPHLPRAL
MRSTTKTS
IgE_CH3 containing IgG FcγR-binding Loop 1 +
Loop 3:
(SEQ ID NO: 19)
DSNPRGVSAYLSRPSPFDLFIRKSPTITCLVVDVSHEDPEVNLTWSRASG
KPVNHSTRKEEKQRNGTLTVTSTLPVGTRDWIEGETYQCRVTNKALPAPL
MRSTTKTS
IgE_CH3 containing IgG FcγR-binding Loop 2 +
Loop 3:
(SEQ ID NO: 20)
DSNPRGVSAYLSRPSPFDLFIRKSPTITCLVVDLAPSKGTVNLTWSRASG
KPVNHSTRKEEKQYNSTYRVTSTLPVGTRDWIEGETYQCRVTNKALPAPL
MRSTTKTS
IgE_CH3 containing IgG FcγR Loop 1 + Loop 2 +
Loop 3:
(SEQ ID NO: 22)
DSNPRGVSAYLSRPSPFDLFIRKSPTITCLVVDVSHEDPEVNLTWSRASG
KPVNHSTRKEEKQYNSTYRVTSTLPVGTRDWIEGETYQCRVTNKALPAPL
MRSTTKTS

Sequences for the fusion proteins were as follows. Each fusion protein further comprises wild-type IgE_VH, IgE_CH1, IgE_CH2 and IgE_CH3 (i.e. SEQ ID NO:s 1, 2, 3 and 4):

IgE_CH4 plus IgG1 Hinge-CH2
(containing RS linker):
(SEQ ID NO: 23)
GPRAAPEVYAFATPEWPGSRDKRTLACLIQNFMPEDISVQWLHNEVQLPD
ARHSTTQPRKTKGSGFFVFSRLEVTRAEWEQKDEFICRAVHEAASPSQTV
QRAVSVNPGKRSEPKSCDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAK
IgE_CH4 plus IgG1 Hinge-CH2-CH3
(containing RS linker)
(SEQ ID NO: 24)
GPRAAPEVYAFATPEWPGSRDKRTLACLIQNFMPEDISVQWLHNEVQLPD
ARHSTTQPRKTKGSGFFVFSRLEVTRAEWEQKDEFICRAVHEAASPSQTV
QRAVSVNPGKRSEPKSCDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK

The full amino acid sequence of the heavy chain of the IgE plus IgG1 Hinge-CH2 construct is shown below:

(SEQ ID NO: 25)
EVQLVESGGGLVQPGGSLRLSCAASGFNKKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGKRS
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAK

The full amino acid sequence of the heavy chain of the IgE plus IgG1 Hinge-CH2-CH3 construct is shown below:

(SEQ ID NO: 26)
EVQLVESGGGLVQPGGSLRLSCAASGFNKKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGKRS
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK

All constructs were confirmed by sequencing. DNA was prepared and transiently transfected into CHO cells using the MaxCyte STX® electroporation system (MaxCyte Inc., Gaithersburg, USA) with OC-400 processing assemblies. 7-10 days post transfection, the supernatants were harvested.

Antibodies (i.e. comprising the variant heavy chains described above and kappa light chains derived from trastuzumab IgE) were purified from cell culture supernatant using either CaptureSelect™ IgE Affinity Matrix (ThermoFisher, Loughborough, UK) or Mab Select Sure columns (GE Healthcare, Little Chalfont, UK) for the IgG1 CH2-CH3 fusion. Eluted fractions were buffer exchanged into PBS and filter sterilised before quantification by A_{280 nm} using an extinction coefficient (E_{c (0.1%)}) based on the predicted amino acid sequence.

A fusion protein comprising IgE including IgG1 Hinge-CH2-CH3 (SEQ ID NOs:24 and 26; see FIG. 2A) was purified using a 1 mL Mab Select Prism A™ column to yield 11 mg of total protein (˜2.7 ml volume at 4.09 mg/ml; see FIG. 2B). SDS-PAGE was carried out in which 1 protein was added to each lane (see FIG. 2C).

### Example 2—Binding of Fusion Protein to CD64 (FcγRI)

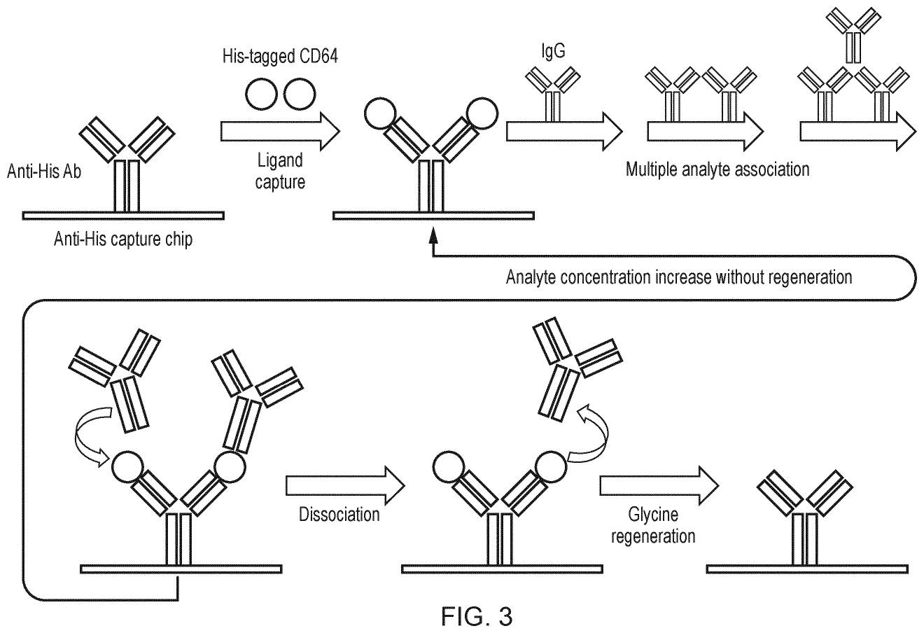

To accurately determine the kinetics of the fusion protein to CD64 (FcγRI), single cycle kinetic analysis was performed on purified antibodies. The principle of the assay is shown in FIG. 3. Kinetic experiments were performed on a Biacore T200 (serial no. 1909913) running Biacore T200 Control software V2.0.1 and Evaluation software V3.0 (GE Healthcare, Uppsala, Sweden). All single cycle kinetic experiments were run at 25° C. with HBS-P+ running buffer (pH 7.4) (GE Healthcare, Little Chalfont, UK).

At the start of each cycle, His-tagged CD64 diluted in running buffer (HBS-P+ buffer) to a final concentration was loaded on to an anti-HIS capture chip (CM5 coupled with ˜9000 RU anti-His antibody (Cat No. 28995056) using standard amine chemistry; GE Healthcare, Little Chalfont, UK) to ˜60 RU at a flow rate of 30 μl/min. The surface was then allowed to stabilise. Single cycle kinetic data was obtained with purified antibody as the analyte at a flow rate of 30 μl/min to minimise any potential mass transport limitations. A five point, three-fold dilution range from 0.411 nM to 33.33 nM of antibody without regeneration between each concentration was used. The association phase for the five injections of increasing concentrations of antibody was monitored for 200 seconds each time and a single dissociation phase was measured for 300 seconds following the last injection analyte. Regeneration of the anti-HIS capture surface was conducted using two injections of 10 mM Glycine-HCl pH 1.5. The signal from the reference channel F_{c}1 was subtracted from that of F_{c}2 to correct for differences in non-specific binding to a reference surface, and a global Rₘₐₓ parameter was used in the 1-to-1 binding model. FIG. 6 is a schematic diagram of the scientific principle behind the assay.

Langmuir (1:1) binding analysis was the model selected for kinetic evaluation. The model describes a 1:1 interaction at the surface:

$$A + B {⇌}_{{k}_{d}}^{{k}_{a}} AB \\ {K}_{D} = \frac{{k}_{d}}{{k}_{a}}$$

where: kₐ is the association rate constant (M⁻¹s⁻¹); and
k_{d} is the dissociation rate constant (s⁻¹)

The closeness of data fit is judged in terms of the Chi square value which describes the deviation between the experimental and fitted curves:

$$Chi square = \frac{\sum {( {r}_{f} - {r}_{x} )}^{2}}{n - p}$$

where: r_{f} is the fitted value at a given point;
rₓ is the experimental value at the same point;
n is the number of data points; and
p is the number of fitted parameters

The fitting algorithm seeks to minise Chi square.

Results

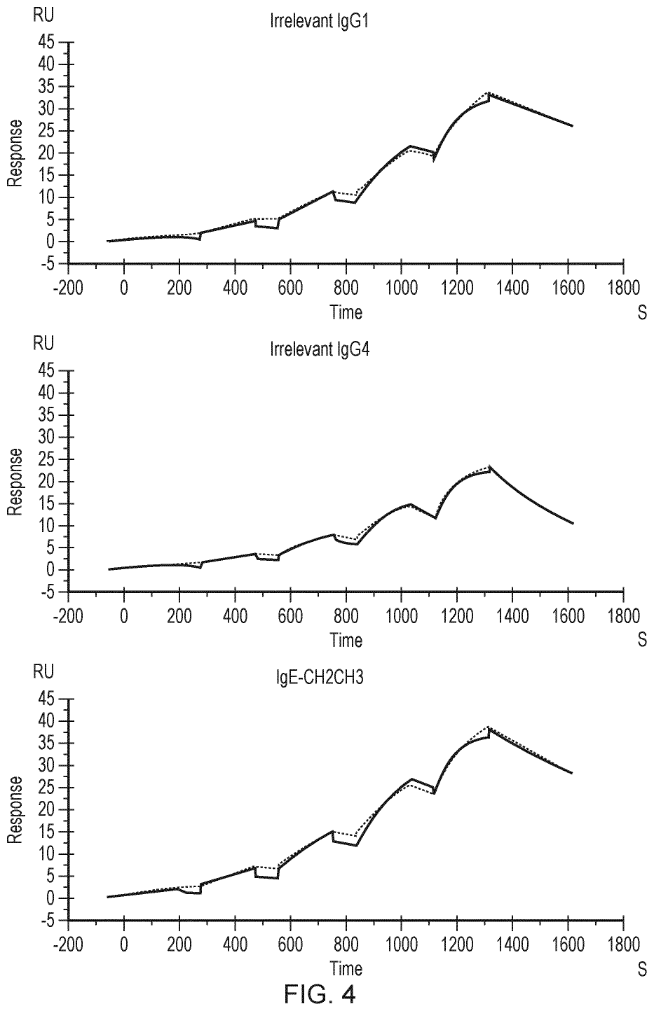

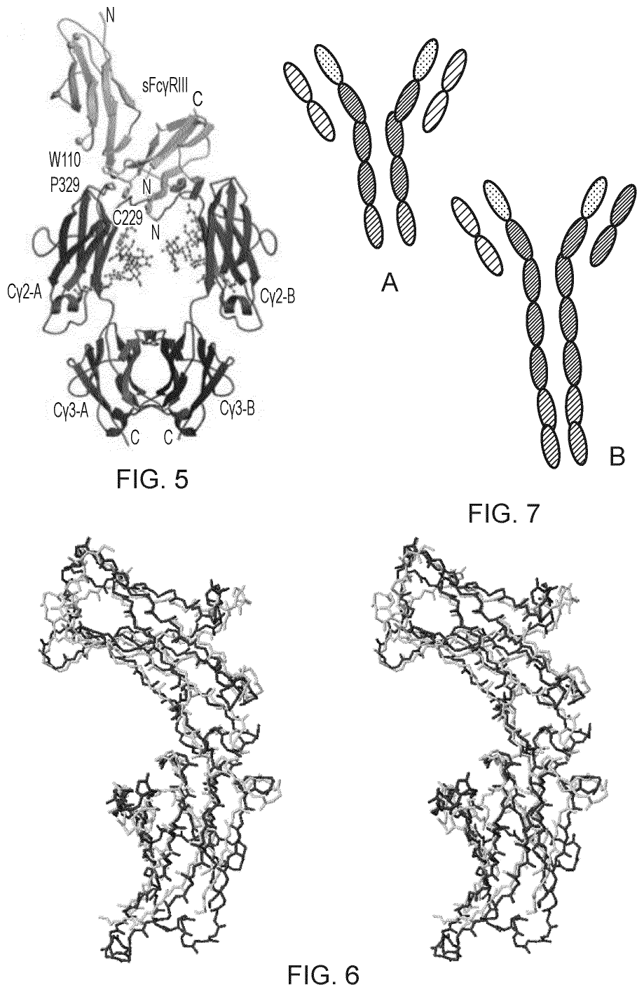

As shown in FIG. 4 and Table 4 below, the binding of IgE-CH2CH3 (SEQ ID NO:26) to FcγRI (CD64) is similar to that of wild type IgG.

TABLE 4

| Antibody | Kₐ (1/Ms) | K_{d} (1/s) | K_{D} (M) | R_{MAX} (RU) | Chi² (RU²) | Relative Binding |
|---|---|---|---|---|---|---|
| Irrelevant IgG1 | 3.19E+05 | 8.30E−04 | 2.61E−09 | 37.5 | 0.446 | ++++ |
| Irrelevant IgG4 | 4.17E+05 | 2.49E−03 | 5.97E−09 | 27.5 | 0.189 | ++++ |
| IgE-CH2CH3 | 4.26E+05 | 1.00E−03 | 2.35E−09 | 41.3 | 0.853 | ++++ |

### Example 3—Binding of Hybrid IgE Variants

To test binding of hybrid IgE variants to the high affinity FcγRI (CD64) and low affinity FcγRIIIA (CD16A) receptors, wild type IgE was used as a negative control and CHO supernatants were screed prior to variant selection and purification.

FIG. 8 is a schematic diagram illustrating the assay steps in which IgE in the supernatant was captured on the Biacore chip using CaptureSelect biotin Anti-IgE bound to a streptavidin chip. Only Fc2 was used for the capture with Fc1 used as the reference.

Antibodies were loaded to the same level. A single injection of CD64 (25 nM) and CD16A (1 μM) was used. The concentrations used were based on the affinity of binding to IgG1.

CD64 Single Cycle Kinetic Biacore™ Analysis of Purified Proteins

To accurately determine the kinetics of select variants to CD64, single cycle kinetic analysis was performed on purified antibodies. Kinetic experiments were performed on a Biacore T200 (serial no. 1909913) running Biacore T200 Control software V2.0.1 and Evaluation software V3.0 (GE Healthcare, Uppsala, Sweden). All single cycle kinetic experiments were run at 25° C. with HBS-P+ running buffer (pH 7.4) (GE Healthcare, Little Chalfont, UK).

At the start of each cycle, His-tagged CD64 diluted in running buffer (HBS-P+ buffer supplemented with 150 mM NaCl) to a final concentration was loaded on to an anti-HIS capture chip (GE Healthcare, Little Chalfont, UK) to ˜60 RU or ˜20 RU at a flow rate of 10 μl/min. The surface was then allowed to stabilise. Single cycle kinetic data was obtained with purified antibody as the analyte at a flow rate of 30 μl/min to minimise any potential mass transport limitations. A five point, three-fold dilution range from 0.411 nM to 33.33 nM of antibody without regeneration between each concentration was used. The association phase for the five injections of increasing concentrations of antibody was monitored for 200 seconds each time and a single dissociation phase was measured for 300 seconds following the last injection analyte. Regeneration of the anti-HIS capture surface was conducted using two injections of 10 mM Glycine-HCl pH 1.5. The signal from the reference channel F_{c}1 was subtracted from that of F_{c}2 to correct for differences in non-specific binding to a reference surface, and a global Rₘₐₓ parameter was used in the 1-to-1 binding model.

Biacore™ Screening (CD64 and CD16A (176 Val)):

To assess the binding of all variants to CD64 (Sino Biological Cat. No. CT009-H08H) and CD16A (176 Val) (Sino Biological cat. no. 10389-H08H1), Biacore kinetic analysis at a single concentration was performed on supernatants from transfected CHO cell cultures. Kinetic experiments were performed on a Biacore T200 (serial no. 1909913) running Biacore T200 Control software V2.0.1 and Evaluation software V3.0 (GE Healthcare, Uppsala, Sweden). All kinetic experiments were run at 25° C. with HBS-EP+ running buffer (pH 7.4) (GE Healthcare, Little Chalfont, UK). Antibodies were loaded onto Fc2 of the Straptavidin chip (GE Healthcare, Little Chalfont, UK) preloaded with CaptureSelect Biotin Anti-IgE (Thermo Cat. No. 7103542500). Antibodies were captured at a flow rate of 10 μl/min to give an immobilisation level (RL) of ˜400 RU. Binding data was obtained with either CD64 at 25 nM for 150 seconds or CD16A (176 Val) at 1 μM for 30 seconds as the analyte at a flow rate of 10 μl/min. The signal from the reference channel F_{c}1 (no antibody) was subtracted from that of F_{c}2 to correct for differences in non-specific binding to a reference surface. Regeneration of the anti-IgE capture surface was conducted using one injection of glycine pH 2.0.

Results

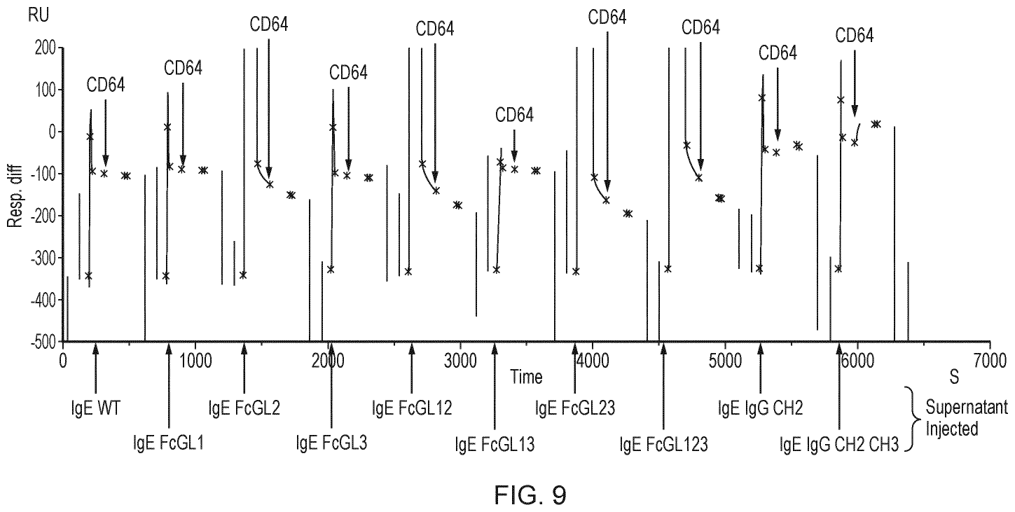

FIG. 9 shows the results from a manual run for 25 nM CD64 (FcγRI). As can be seen, CaptureSelect Biotin Anti-IgE Conjugate was able to bind all of the antibody variants tested, suggesting that the receptor does not bind to an epitope present on any of the loops swapped out. However, antibody variants containing Loop 2 (in green) appeared to be less stably bound. Only the IgE fusions (SEQ ID NO:s 25 and 26) containing IgG CH2 and IgG CH2-CH3 domains were able to bind to CD64, although the off-rate for the fusion containing only CH2 appeared to be much faster.

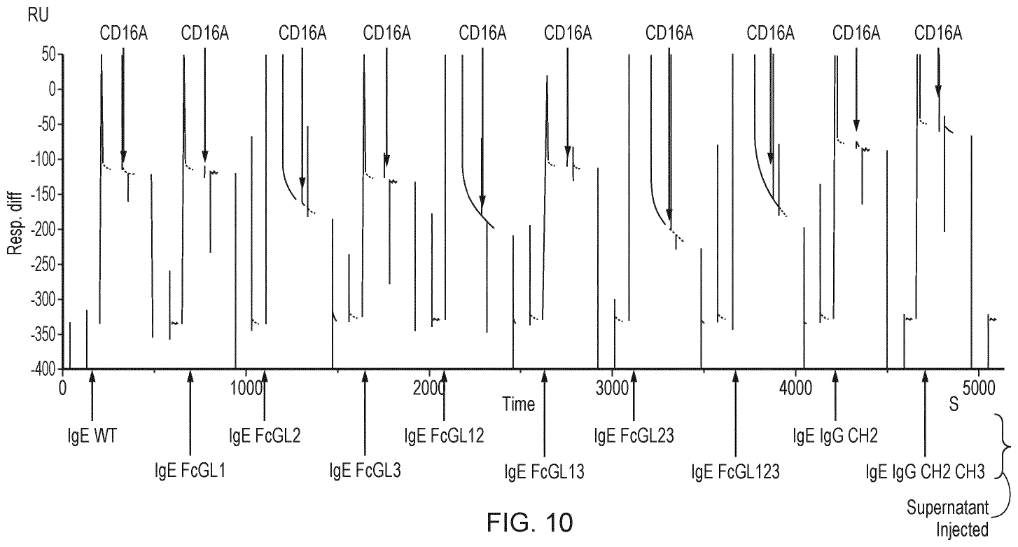

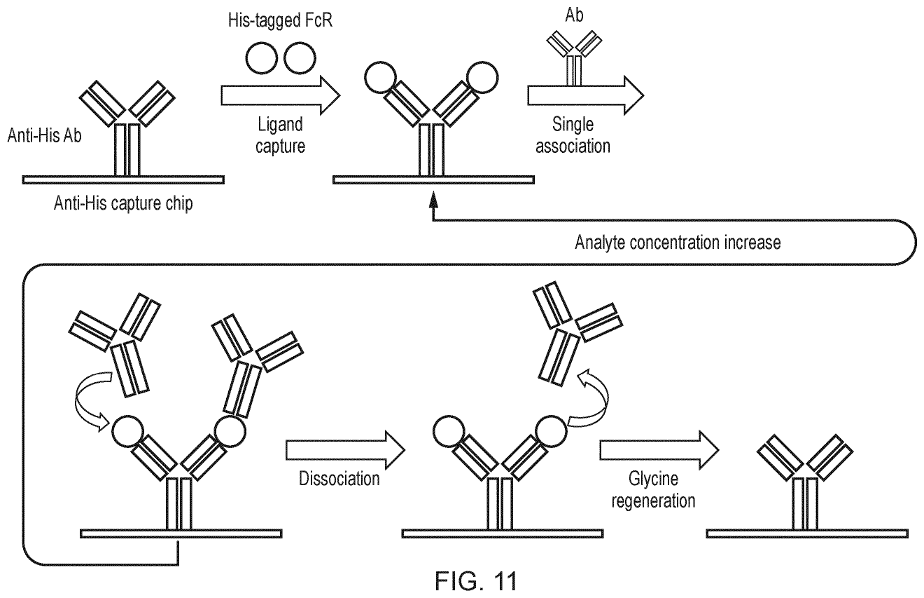

FIG. 10 shows the results from a manual run for 1 μM CD16A (FcRγIIIA) (176 Val). The figure shows that, under the specific conditions of the experiment, only the IgE fusion protein with IgG CH2-CH3 domains (SEQ ID NO:26) appeared able to bind CD16A.

In further studies, IgE-CH2-CH3 can be compared to IgG1 and IgE against a full panel of Fcγ and Fcε receptors using similar techniques.

### Example 4—Binding to Fc Epsilon RI Alpha (FcεRIα)

The aim of this experiment was to investigate the binding of purified wild type IgE and IgE CH2-CH3 to FcεRIα. Herceptin (Trastuzumab) was used as a control. The principle of the assay is shown in FIG. 11.

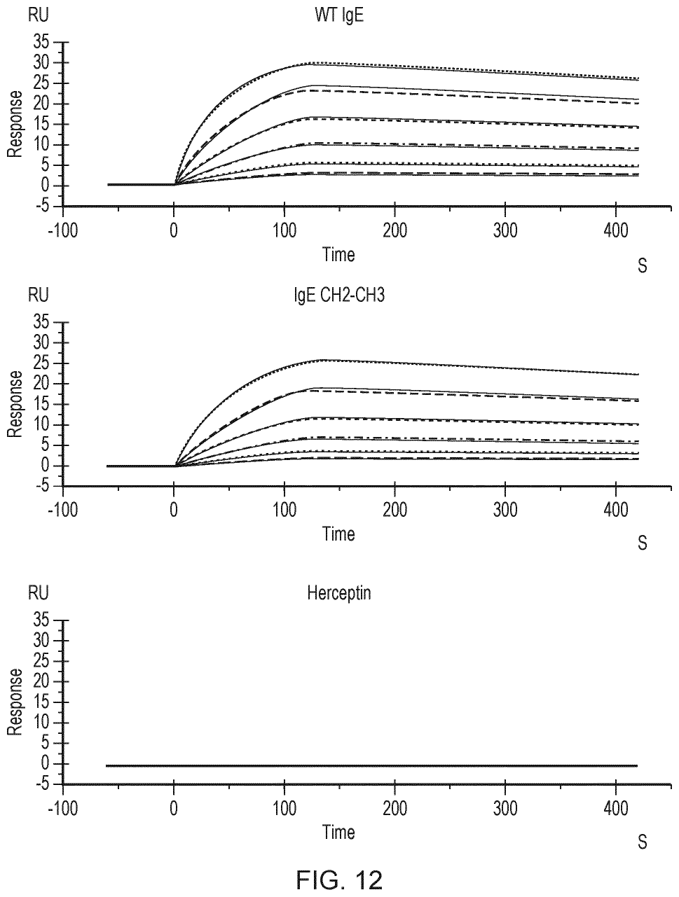

As shown in FIG. 12 and Table 5 below, wild type IgE and IgE CH2-CH3 (SEQ ID NO:26) bound similarly to the FcεRIα receptor. No binding of Herceptin to FcεRIα was observed.

TABLE 5

| | Kₐ | K_{d} | K_{D} | R_{MAX} | Chi² |
|---|---|---|---|---|---|
| Antibody | (1/Ms) | (1/s) | (M) | (RU) | (RU²) |
| WT IgE | 5.21E+05 | 4.69E−04 | 9.00E−10 | 31.6 | 0.164 |
| Herceptin | — | — | — | — | — |
| IgE−CH2CH3 | 3.52E+05 | 4.62E−04 | 1.31E−09 | 30.1 | 0.0307 |
| IgE 3His | 4.16E+05 | 5.13E−04 | 1.23E−09 | 30.8 | 0.0732 |

The studies above show that the variant IgE antibodies comprising IgG CH2 and CH3 domains bind to gamma and epsilon Fc receptors. In further studies, the antibodies can be assessed for recruitment of both IgG and IgE effector cells for tumour cell killing in vitro. An in vivo comparison of hybrid IgE vs wild type IgE vs IgG can also be performed.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

### Example 5—Anti-HMW-MAA Hybrid Antibody

In a further example, another IgE variant is created in which the IgG hinge and IgG CH2-CH3 domain pair is fused to the IgE framework at the C terminus (as in Example 1). The IgE antibody is based on an anti-HMW-MAA antibody, for example, as disclosed in WO 2013/050725.

Another anti-HMW-MAA IgE variant is created in which the IgG hinge and CH2 domain is fused to the C terminus of an anti-HMW-MAA antibody.

Further variant anti-HMW-MAA IgE antibodies are generated in which one or more loops in a Cε3 domain of the IgE are replaced by one or more FcγR-binding loops derived from a Cγ2 domain of an IgG antibody. The loops that are replaced in the Cε3 domain of the IgE show structural homology to the FcγR-binding loops in the Cγ2 domain of IgG.

The antibodies are produced and purified as described in Example 1. Analysis of antibody binding is tested as described in Examples 2-4.

The sequences for a HMW-MAA IgE are as follows:

HMW-MAA VH (SEQ ID NO: 161):
EQVKLQQSGGGLVQPGGSMKLSCVVSGFTFSNYWMNWVRQSPEKGLEWIA
EIRLKSNNFGRYYAESVKGRFTISRDDSKSSAYLQMINLRAEDTGIYYCT
SYGNYVGHYFDHWGQGTTVTVSS
HMW-MAA VL (SEQ ID NO: 162):
DIELTQSPKFMSTSVCDRVSVTCKASQNVDTNVAWYQQKPGQSPEPLLFS
ASYRYTGVPDRFTGSGSGTDFTLTISNVQSEDLAEYFCQQYNSYPLTFGG
GTKLEIK

Alternative variable domain sequences for a HMW_MAA IgE are as follows:

HMW-MAA VH Alternative
(SEQ ID NO: 177)
EVQLVQSGGGLVQPGGSLKLSCAVSGFTFSNYWMNWVRQAPGKGLEWVGE
IRLKSNNFGRYYAESVKGRFTISRDDSKNTAYLQMNSLKTEDTAVYYCTS
YGNYVGHYFDHWGQGTLVTVSS
HMW-MAA VL Alternative
(SEQ ID NO: 178)
DIQLTQSPSFLSASVGDRVTITCKASQNVDTNVAWYQQKPGKAPKPLLFS
ASYRYTGVPSRFSGSGSGTDFTLTISSLQPEDFATYFCQQYNSYPLTFGG
GTKVEIK

The constant domain sequences of the HMW-MAA IgE antibodies (comprising an IgG hinge and IgG CH2-CH3 domain (or IgG CH2 domain) fused to the IgE framework) are as shown above in Example 1, i.e. SEQ ID Nos: 2 to 4 plus SEQ ID NO:23 or SEQ ID NO:24.

### Example 6—Production of a Heterodimeric IgE

Construction of IgE-IgG-Fc (IGEG) Fusion Proteins

DNA sequences corresponding to the WT IgE constant domain were codon optimised for CHO expression and synthesised (GeneArt, ThermoFisher Scientific, Loughborough, UK) with flanking restriction enzyme sites for cloning into a pANT dual Ig expression vector system for human heavy and kappa light chains. The heavy chain, also containing Trastuzumab VH, was cloned between the Mlu I and Kpn I restriction sites. Trastuzumab Vk, synthesised separately, was cloned between the BssH II and BamH I restriction sites, upstream of the kappa constant region.

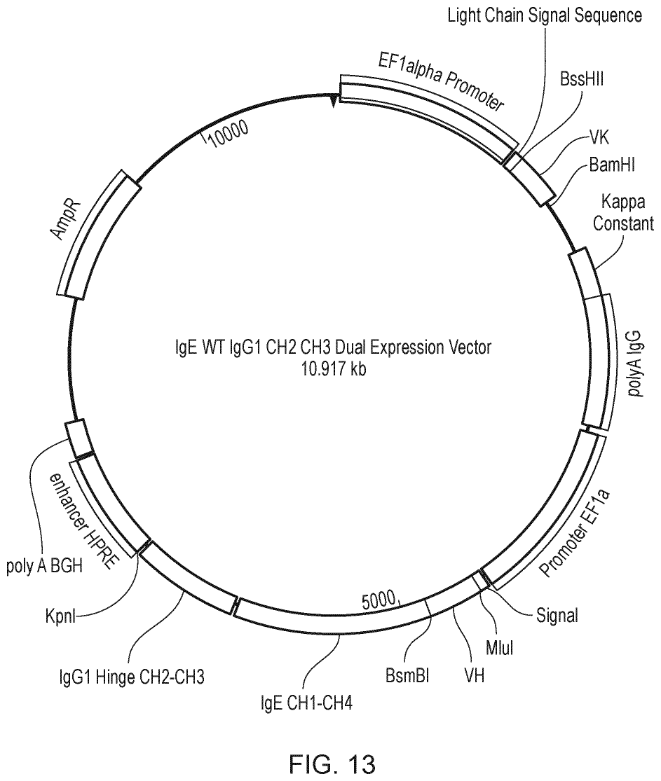

In order to generate the IgE-IgG (IGEG) fusion, specific primers were used to amplify WT IgE whilst removing the stop codon at the end of IgE CH4, and in a separate reaction to amplify IgG1 Hinge-CH2-CH3 synthesised separately. Pull-through PCR was used to combine both fragments and introduce Mlu I and KpnI restriction sites for cloning into the dual expression vector. A BsmBI restriction site was subsequently introduced by site directed mutagenesis (Quikchange, Agilent) within the FW4 region of the Trastuzumab VH which, along with Mlu I, permitted swapping of VH regions (See FIG. 13 for a diagram of the vector).

To remove a potential free cysteine residue within the IgG hinge region, primers were designed to introduce the Cys220Ser amino acid substitutions (numbering is based upon the EU numbering scheme with reference to the IgG portion of the IGEG sequence) by site directed mutagenesis using the BsmBI-containing IgE-IgG construct as template. The Cys220Ser mutation is indicated in blue in the sequences below.

To remove the ability of the IgG portion of the IGEG to bind to FcRn, amino acid substitutions were made at three residues normally involved in FcRn binding, Ile253Ala, His310Ala and His435Ala (numbering is based upon the EU numbering scheme with reference to the IgG portion of the IGEG sequence). Primers were designed and site directed mutagenesis (Agilent Quikchange) performed using the BsmBI-containing IgE-IgG constructs (containing either Cys220 or Ser220) as template.

In order to generate the HMW-MAA (CSPG4) series of constructs, the HMW-MAA VH and VK were synthesised (GeneArt) and cloned into the IGEG vectors. The HMW-MAA VH was cloned between the MluI and BsmBI restriction sites, and the HMW-MAA Vk was cloned between the BssH II and BamH I restriction sites.

All constructs were confirmed by Sanger sequencing.

The sequences were as follows (underlining shows variable domain sequences, standard text shows IgE Fc sequences, italic shows IgG-derived sequences, bold shows specific mutations):

Trastuzumab IgE/IGEG Variant Sequences

Trastuzumab IgE Heavy Chain
(SEQ ID NO: 179)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTQSPSVFPLTRCCKNIPSNATSVTLGCLATGYFPEPVMVTWDTGSL
NGTTMTLPATTLTLSGHYATISLLTVSGAWAKQMFTCRVAHTPSSTDWVDNKTFSV
CSRDFTPPTVKILQSSCDGGGHFPPTIQLLCLVSGYTPGTINITWLEDGQVMDVDLST
ASTTQEGELASTQSELTLSQKHWLSDRTYTCQVTYQGHTFEDSTKKCADSNPRGVS
AYLSRPSPFDLFIRKSPTITCLVVDLAPSKGTVNLTWSRASGKPVNHSTRKEEKQRNG
TLTVTSTLPVGTRDWIEGETYQCRVTHPHLPRALMRSTTKTSGPRAAPEVYAFATPE
WPGSRDKRTLACLIQNFMPEDISVQWLHNEVQLPDARHSTTQPRKTKGSGFFVFSRL
EVTRAEWEQKDEFICRAVHEAASPSQTVQRAVSVNPGK
Trastuzumab IgE-IgG-Fc Heavy Chain
(SEQ ID NO: 163)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTQSPSVFPLTRCCKNIPSNATSVTLGCLATGYFPEPVMVTWDTGSL
NGTTMTLPATTLTLSGHYATISLLTVSGAWAKQMFTCRVAHTPSSTDWVDNKTFSV
CSRDFTPPTVKILQSSCDGGGHFPPTIQLLCLVSGYTPGTINITWLEDGQVMDVDLST
ASTTQEGELASTQSELTLSQKHWLSDRTYTCQVTYQGHTFEDSTKKCADSNPRGVS
AYLSRPSPFDLFIRKSPTITCLVVDLAPSKGTVNLTWSRASGKPVNHSTRKEEKQRNG
TLTVTSTLPVGTRDWIEGETYQCRVTHPHLPRALMRSTTKTSGPRAAPEVYAFATPE
WPGSRDKRTLACLIQNFMPEDISVQWLHNEVQLPDARHSTTQPRKTKGSGFFVFSRL
EVTRAEWEQKDEFICRAVHEAASPSQTVQRAVSVNPGKRSEPKSCDKTHTCPPCPAP
ELLGGPSVFLFPPKPKDTLMISRTPEVTCVVVDVSHEDPEVKFNWYVDGVEVHNAKTKPR
EEQYNSTYRWSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
Trastuzumab IgE-IgG-Fc C220S Heavy Chain
(SEQ ID NO: 164)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTQSPSVFPLTRCCKNIPSNATSVTLGCLATGYFPEPVMVTWDTGSL
NGTTMTLPATTLTLSGHYATISLLTVSGAWAKQMFTCRVAHTPSSTDWVDNKTFSV
CSRDFTPPTVKILQSSCDGGGHFPPTIQLLCLVSGYTPGTINITWLEDGQVMDVDLST
ASTTQEGELASTQSELTLSQKHWLSDRTYTCQVTYQGHTFEDSTKKCADSNPRGVS
AYLSRPSPFDLFIRKSPTITCLVVDLAPSKGTVNLTWSRASGKPVNHSTRKEEKQRNG
TLTVTSTLPVGTRDWIEGETYQCRVTHPHLPRALMRSTTKTSGPRAAPEVYAFATPE
WPGSRDKRTLACLIQNFMPEDISVQWLHNEVQLPDARHSTTQPRKTKGSGFFVFSRL
EVTRAEWEQKDEFICRAVHEAASPSQTVQRAVSVNPGKRSEPKS [symbol] DKTHTCPPCPAP
ELLGGPSVFLFPPKPKDTLMISRTPEVTCVVVDVSHEDPEVKFNWYVDGVEVHNAKTKPR
EEQYNSTYRWSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
Trastuzumab IgG-IgG-Fc dFcRn Heavy Chain
(SEQ ID NO: 165)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTQSPSVFPLTRCCKNIPSNATSVTLGCLATGYFPEPVMVTWDTGSL
NGTTMTLPATTLTLSGHYATISLLTVSGAWAKQMFTCRVAHTPSSTDWVDNKTFSV
CSRDFTPPTVKILQSSCDGGGHFPPTIQLLCLVSGYTPGTINITWLEDGQVMDVDLST
ASTTQEGELASTQSELTLSQKHWLSDRTYTCQVTYQGHTFEDSTKKCADSNPRGVS
AYLSRPSPFDLFIRKSPTITCLVVDLAPSKGTVNLTWSRASGKPVNHSTRKEEKQRNG
TLTVTSTLPVGTRDWIEGETYQCRVTHPHLPRALMRSTTKTSGPRAAPEVYAFATPE
WPGSRDKRTLACLIQNFMPEDISVQWLHNEVQLPDARHSTTQPRKTKGSGFFVFSRL
EVTRAEWEQKDEFICRAVHEAASPSQTVQRAVSVNPGKRSEPKSCDKTHTCPPCPAP
ELLGGPSVFLFPPKPKDTLM [symbol] SRTPEVTCVVVDVSHEDPEVKFNWYVDGVEVHNAKTKP
REEQYNSTYRWSVLTVL [symbol] QDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVD
KSRWQQGNVFSCSVMHEALHN [symbol] YTQKSLSLSPGK
Trastuzumab IgG-IgG-Fc dFcRn C220S Heavy Chain
(SEQ ID NO: 166)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTQSPSVFPLTRCCKNIPSNATSVTLGCLATGYFPEPVMVTWDTGSL
NGTTMTLPATTLTLSGHYATISLLTVSGAWAKQMFTCRVAHTPSSTDWVDNKTFSV
CSRDFTPPTVKILQSSCDGGGHFPPTIQLLCLVSGYTPGTINITWLEDGQVMDVDLST
ASTTQEGELASTQSELTLSQKHWLSDRTYTCQVTYQGHTFEDSTKKCADSNPRGVS
AYLSRPSPFDLFIRKSPTITCLVVDLAPSKGTVNLTWSRASGKPVNHSTRKEEKQRNG
TLTVTSTLPVGTRDWIEGETYQCRVTHPHLPRALMRSTTKTSGPRAAPEVYAFATPE
WPGSRDKRTLACLIQNFMPEDISVQWLHNEVQLPDARHSTTQPRKTKGSGFFVFSRL
EVTRAEWEQKDEFICRAVHEAASPSQTVQRAVSVNPGKRSEPKSSDKTHTCPPCPAP
ELLGGPSVFLFPPKPKDTLM [symbol] SRTPEVTCVVVDVSHEDPEVKFNWYVDGVEVHNAKTKP
REEQYNSTYRWSVLTVL [symbol] QDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVD
KSRWQQGNVFSCSVMHEALHN [symbol] YTQKSLSLSPGK
Kappa Trastuzumab Light Chain
(SEQ ID NO: 167)
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC

HMW-MAA IgE/IGEG Variant Sequences

HMW-MAA IgE Heavy Chain
(SEQ ID NO: 168)
EVQLVQSGGGLVQPGGSLKLSCAVSGFTFSNYWMNWVRQAPGKGLEWVGEIRLKS
NNFGRYYAESVKGRFTISRDDSKNTAYLQMNSLKTEDTAVYYCTSYGNYVGHYFD
HWGQGTLVTVSSASTQSPSVFPLTRCCKNIPSNATSVTLGCLATGYFPEPVMVTWDT
GSLNGTTMTLPATTLTLSGHYATISLLTVSGAWAKQMFTCRVAHTPSSTDWVDNKT
FSVCSRDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKCADSNPRG
VSAYLSRPSPFDLFIRKSPTITCLVVDLAPSKGTVNLTWSRASGKPVNHSTRKEEKQR
NGTLTVTSTLPVGTRDWIEGETYQCRVTHPHLPRALMRSTTKTSGPRAAPEVYAFAT
PEWPGSRDKRTLACLIQNFMPEDISVQWLHNEVQLPDARHSTTQPRKTKGSGFFVFS
RLEVTRAEWEQKDEFICRAVHEAASPSQTVQRAVSVNPGK
HMW-MAA IgE IgG-Fc Heavy Chain
(SEQ ID NO: 169)
EVQLVQSGGGLVQPGGSLKLSCAVSGFTFSNYWMNWVRQAPGKGLEWVGEIRLKS
NNFGRYYAESVKGRFTISRDDSKNTAYLQMNSLKTEDTAVYYCTSYGNYVGHYFD
HWGQGTLVTVSSASTQSPSVFPLTRCCKNIPSNATSVTLGCLATGYFPEPVMVTWDT
GSLNGTTMTLPATTLTLSGHYATISLLTVSGAWAKQMFTCRVAHTPSSTDWVDNKT
FSVCSRDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKCADSNPRG
VSAYLSRPSPFDLFIRKSPTITCLVVDLAPSKGTVNLTWSRASGKPVNHSTRKEEKQR
NGTLTVTSTLPVGTRDWIEGETYQCRVTHPHLPRALMRSTTKTSGPRAAPEVYAFAT
PEWPGSRDKRTLACLIQNFMPEDISVQWLHNEVQLPDARHSTTQPRKTKGSGFFVFS
RLEVTRAEWEQKDEFICRAVHEAASPSQTVQRAVSVNPGKRSEPKSCDKTHTCPPCP
APELLGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
HMW-MAA IgE-IgG-Fc C220S Heavy Chain
(SEQ ID NO: 170)
EVQLVQSGGGLVQPGGSLKLSCAVSGFTFSNYWMNWVRQAPGKGLEWVGEIRLKS
NNFGRYYAESVKGRFTISRDDSKNTAYLQMNSLKTEDTAVYYCTSYGNYVGHYFD
HWGQGTLVTVSSASTQSPSVFPLTRCCKNIPSNATSVTLGCLATGYFPEPVMVTWDT
GSLNGTTMTLPATTLTLSGHYATISLLTVSGAWAKQMFTCRVAHTPSSTDWVDNKT
FSVCSRDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKCADSNPRG
VSAYLSRPSPFDLFIRKSPTITCLVVDLAPSKGTVNLTWSRASGKPVNHSTRKEEKQR
NGTLTVTSTLPVGTRDWIEGETYQCRVTHPHLPRALMRSTTKTSGPRAAPEVYAFAT
PEWPGSRDKRTLACLIQNFMPEDISVQWLHNEVQLPDARHSTTQPRKTKGSGFFVFS
RLEVTRAEWEQKDEFICRAVHEAASPSQTVQRAVSVNPGKRSEPKS [symbol] DKTHTCPPCP
APELLGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
HMW-MAA IgG-IgG-Fc dFcRn Heavy Chain
(SEQ ID NO: 171)
EVQLVQSGGGLVQPGGSLKLSCAVSGFTFSNYWMNWVRQAPGKGLEWVGEIRLKS
NNFGRYYAESVKGRFTISRDDSKNTAYLQMNSLKTEDTAVYYCTSYGNYVGHYFD
HWGQGTLVTVSSASTQSPSVFPLTRCCKNIPSNATSVTLGCLATGYFPEPVMVTWDT
GSLNGTTMTLPATTLTLSGHYATISLLTVSGAWAKQMFTCRVAHTPSSTDWVDNKT
FSVCSRDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKCADSNPRG
VSAYLSRPSPFDLFIRKSPTITCLVVDLAPSKGTVNLTWSRASGKPVNHSTRKEEKQR
NGTLTVTSTLPVGTRDWIEGETYQCRVTHPHLPRALMRSTTKTSGPRAAPEVYAFAT
PEWPGSRDKRTLACLIQNFMPEDISVQWLHNEVQLPDARHSTTQPRKTKGSGFFVFS
RLEVTRAEWEQKDEFICRAVHEAASPSQTVQRAVSVNPGKRSEPKSCDKTHTCPPCP
APELLGGPSVFLFPPKPKDTLM [symbol] SRTPEVTCVVVDVSHEDPEVKFNWYVDGVEVHNAKT
KPREEQYNSTYRVVSVLIVL [symbol] QDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKLT
VDKSRWQQGNVFSCSVMHEALHN [symbol] YTQKSLSLSPGK
HMW-MAA IgG-IgG-Fc dFcRn C220S Heavy Chain
(SEQ ID NO: 172)
EVQLVQSGGGLVQPGGSLKLSCAVSGFTFSNYWMNWVRQAPGKGLEWVGEIRLKS
NNFGRYYAESVKGRFTISRDDSKNTAYLQMNSLKTEDTAVYYCTSYGNYVGHYFD
HWGQGTLVTVSSASTQSPSVFPLTRCCKNIPSNATSVTLGCLATGYFPEPVMVTWDT
GSLNGTTMTLPATTLTLSGHYATISLLTVSGAWAKQMFTCRVAHTPSSTDWVDNKT
FSVCSRDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKCADSNPRG
VSAYLSRPSPFDLFIRKSPTITCLVVDLAPSKGTVNLTWSRASGKPVNHSTRKEEKQR
NGTLTVTSTLPVGTRDWIEGETYQCRVTHPHLPRALMRSTTKTSGPRAAPEVYAFAT
PEWPGSRDKRTLACLIQNFMPEDISVQWLHNEVQLPDARHSTTQPRKTKGSGFFVFS
RLEVTRAEWEQKDEFICRAVHEAASPSQTVQRAVSVNPGKRSEPKS [symbol] DKTHTCPPCP
APELLGGPSVFLFPPKPKDTLM [symbol] SRTPEVTCVVVDVSHEDPEVKFNWYVDGVEVHNAKT
KPREEQYNSTYRVVSVLIVL [symbol] QDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYSKLT
VDKSRWQQGNVFSCSVMHEALHN [symbol] YTQKSLSLSPGK
HMW-MAA Kappa Light Chain
(SEQ ID NO: 173)
DIQLTQSPSFLSASVGDRVTITCKASQNVDTNVAWYQQKPGKAPKPLLFSASYRYTG
VPSRFSGSGSGTDFTLTISSLQPEDFATYFCQQYNSYPLTFGGGTKVEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC

CHO Transient Expression of IgE-IgG (IGEG) Variants

Endotoxin-free DNA encoding the differing IGEG constructs were transiently co-transfected into Freestyle™ CHO-S cells (ThermoFisher, Loughborough, UK) using OC-400 processing assemblies and the MaxCyte STX® electroporation system (MaxCyte Inc., Gaithersburg, USA). Following cell recovery, cells were pooled and diluted at 3×10⁶ cells/mL into CD Opti-CHO medium (ThermoFisher) containing 8 mM L-Glutamine (ThermoFisher) and 1×Hypoxanthine-Thymidine (ThermoFisher). 24 hours post-transfection, the culture temperature was reduced to 32° C. and 30% (of the starting volume) Efficient Feed B (ThermoFisher), 3.3% FunctionMAX™ TiterEnhancer (ThermoFisher) and 1 mM Sodium Butyrate (Sigma, Dorset, UK) were added. Cultures were fed at Day 7 by the addition of 15% (of the current volume) CHO CD Efficient Feed B (ThermoFisher) and 1.65% FunctionMAX™ TiterEnhancer (ThermoFisher). All transfections were cultured for up to 14 days prior to harvesting supernatants.

Purification and Analysis of IGEG Variants

Following culture harvest, antibody supernatants were filtered to remove remaining cell debris and supplemented with 10×PBS to neutralise pH. The majority of IGEG purifications (including dFcRn IGEGs) were performed using IgE CaptureSelect™ affinity resin (ThermoFisher Scientific) in batch binding mode. Affinity resin was equilibrated in PBS pH 7.2, then incubated with each sample for 2 hours at room temperature with rotation followed by a series of PBS washes. All samples were eluted in 50 mM Sodium Citrate, 50 mM Sodium Chloride pH 3.5 and buffered exchanged into PBS pH 7.2. Samples were quantified by OD_{280 nm} using an extinction coefficient (E_{c (0.1%)}) based on the predicted amino acid sequence.

Selected IGEG constructs (e.g. Trastuzumab IGEG containing either Cys220 or Ser220) were purified using Protein A to demonstrate retention of Protein A binding. Following culture harvest, antibody supernatants were filtered to remove remaining cell debris and supplemented with 10×PBS to neutralise pH. Antibodies were then purified from supernatants using 1 mL Hitrap Mab Select PrismA columns (Cytiva, Little Chalfont, UK) previously equilibrated with PBS pH 7.2. Following the sample loading, the columns were washed with PBS pH 7.2 and protein eluted with 0.1 M sodium citrate, pH 3.0. Fractions were collected, and pH adjusted with 1 M Tris-HCl, pH 9.0 followed by buffered exchanged into PBS pH 7.2. Samples were quantified by OD_{280 nm} using an extinction coefficient (E_{c (0.1%)}) based on the predicted amino acid sequence.

All IGEG antibody variants were further purified using a HiLoad™ 26/60 Superdex™ 200 pg preparative SEC column (GE Healthcare, Little Chalfont, UK) using PBS pH 7.2 as the mobile phase. Peak fractions from purifications containing monomeric protein were pooled, concentrated and filter sterilised before quantification by A_{280 nm} using an extinction coefficient (E_{c (0.1%)}) based on the predicted amino acid sequence.

Purified materials were then analysed by analytical SE-HPLC and SDS-PAGE. Analytical SEC was performed using an Acquity UPLC Protein BEH SEC Column, 200 Å, 1.7 μm, 4.6 mm×150 mm (Waters, Elstree, UK) and an Acquity UPLC Protein BEH SEC guard column 30×4.6 mm, 1.7 μm, 200 Å (Waters, Elstree, UK) connected to a Dionex Ultimate 300016 HPLC system (ThermoFisher Scientific, Hemel Hempstead, UK). The method consisted of an isocratic elution over 10 minutes and the mobile phase was 0.2 M potassium phosphate pH 6.8, 0.2 M potassium chloride. The flow rate was 0.35 mL/minute. Detection was carried out by UV absorption at 280 nm. Following purification, all IGEG antibody variants were shown to contain >95% monomeric species.

Single Cycle Kinetic Analysis of IGEG Variants to Cognate Antigen

Binding analysis of HMW-MAA IGEG variants to its cognate antigen by Biacore analysis was not possible due to the lack of conformationally appropriate antigens. Binding was, instead, analysed by flow cytometry.

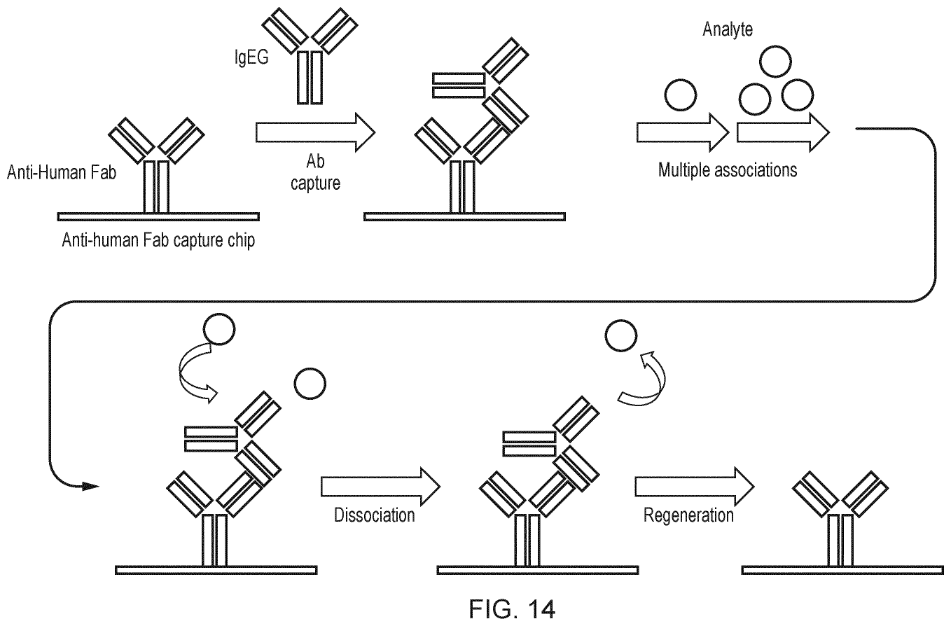

In order to assess the binding of all of the purified Trastuzumab IGEG variants to human Her2 antigen, single cycle kinetic analysis was performed on purified antibodies. Kinetic experiments were performed at 25° C. on a Biacore T200 running Biacore T200 Control software V2.0.1 and Evaluation software V3.0 (Cytiva, Uppsala, Sweden). See FIG. 14 for a schematic of the process.

HBS-EP+(Cytiva, Uppsala, Sweden), supplemented with 1% BSA (Sigma, Dorset, UK) was used as running buffer as well as for ligand and analyte dilutions. Purified antibodies were diluted in running buffer to 10 μg/mL. At the start of each cycle, antibodies were loaded onto F_{c}2, F_{c}3 and F_{c}4 of an anti-Fab (consisting of a mixture of anti-kappa and anti-lambda antibodies) CM5 sensor chip (Cytiva, Little Chalfont, UK). Antibodies were captured at a flow rate of 10 μl/min to give an immobilisation level (R_{L}) of ˜45 RU. The surface was then allowed to stabilise.

Single cycle kinetic data was obtained using recombinant human Her2 antigen (Sino Biological, Beijing, China) as the analyte injected at a flow rate of 40 μl/min to minimise any potential mass transfer effects. A four point, three-fold dilution range from 1.1 nM to 30 nM of antigen in running buffer was used without regeneration between each concentration. The association phases were monitored for 240 seconds for each of the four injections of increasing concentrations of antigen and a single dissociation phase was measured for 600 seconds following the last injection of antigen. Regeneration of the sensor chip surface was conducted using two injections of 10 mM glycine pH 2.1.

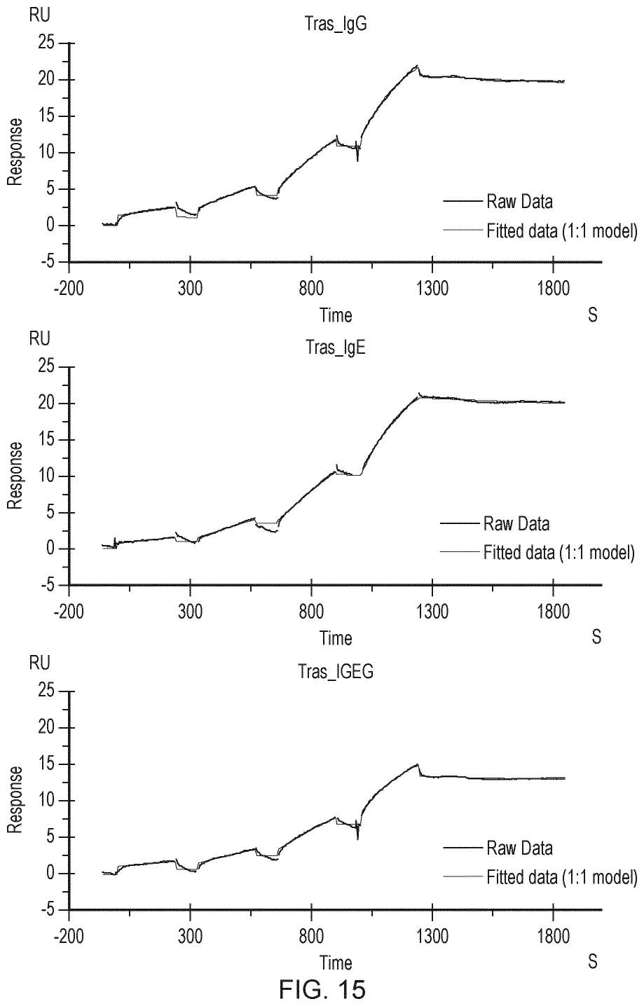

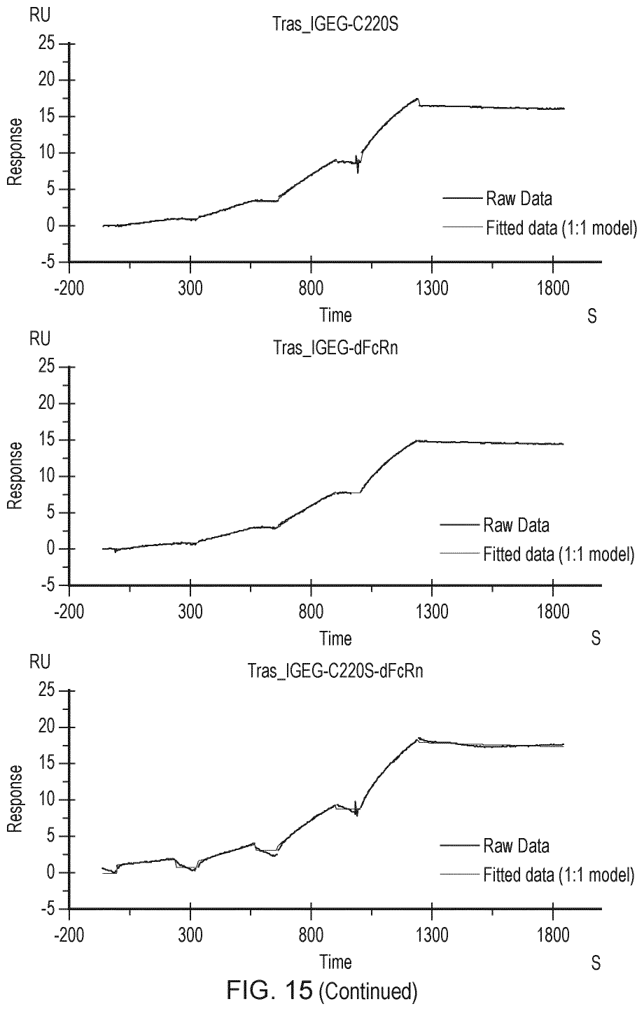

The signal from the reference channel F_{c}1 (no antibody captured) was subtracted from that of F_{c}2, F_{c}3 and F_{c}4 to correct for bulk effect and differences in non-specific binding to a reference surface. The signal from each antibody blank run (antibody captured but no antigen) was subtracted to correct for differences in surface stability (see FIG. 15). Each Trastuzumab construct tested showed similar binding to human Her2 (Table 6).

TABLE 6
Binding parameters of Trastuzumab-IGEG variants to Her2 antigen,
as determined using Biacore single cycle kinetics.

| Antibody | kₐ (1/Ms) | k_{d} (1/s) | K_{D} (M) |
|---|---|---|---|
| Trastuzumab_IgG | 1.72E+05 | 7.81E−05 | 4.54E−10 |
| Trastuzumab_IgE | 2.95E+05 | 7.22E−05 | 2.45E−10 |
| Trastuzumab_IGEG | 1.56E+05 | 5.83E−05 | 3.74E−10 |
| Trastuzumab_IGEG-C220S | 1.69E+05 | 4.82E−05 | 2.85E−10 |
| Trastuzumab_IGEG-dFcRn | 1.64E+05 | 4.16E−05 | 2.53E−10 |
| Trastuzumab_IGEG- | 1.38E+05 | 5.82E−05 | 4.23E−10 |
| C220S-dFcRn | | | |

Assessment of IGEG Variant Binding to human Fc Receptors

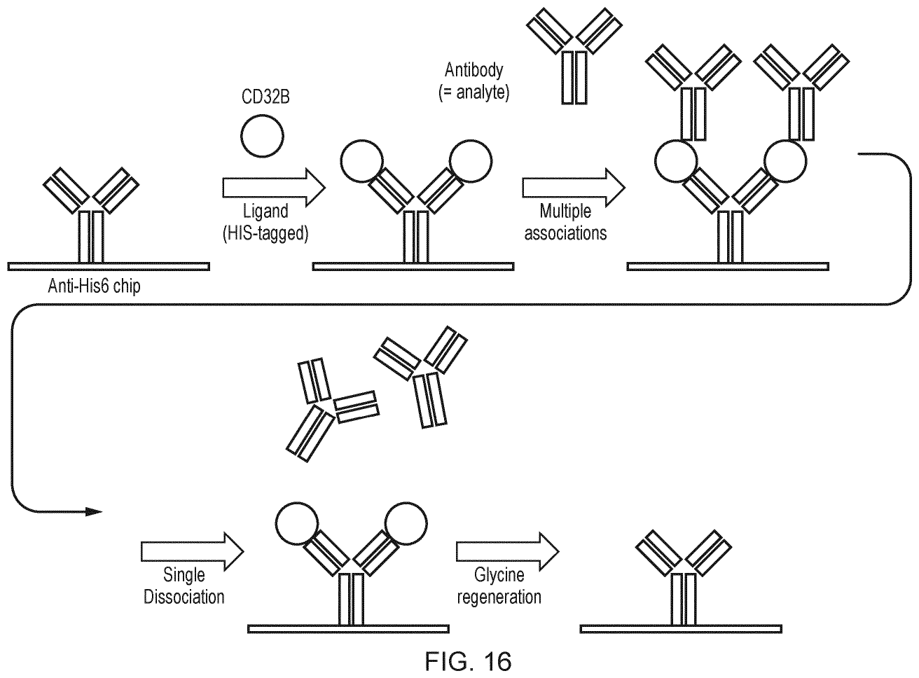

Binding of purified IGEGs to high and low affinity Fc gamma receptors and the high affinity Fc epsilon receptor was assessed by single cycle analysis using a Biacore T200 (serial no. 1909913) instrument running Biacore T200 Evaluation Software V3.0.1 (Uppsala, Sweden) running at a flow rate of 30 μl/min. All of the human Fc gamma receptors (hFcγRI together with the low affinity receptors hFcγRIIIa (both 176F and 176V polymorphisms) and hFcγRIIIb) were obtained from Sino Biological (Beijing, China) and hFcεR1 was obtained from R&D Systems (Minneapolis, USA). FcRs were captured on a CM5 sensor chip pre-coupled using a His capture kit (Cytiva, Uppsala, Sweden) using standard amine chemistry. A schematic detailing the assay used to assess antibody binding to Fc gamma receptors can be found in FIG. 16.

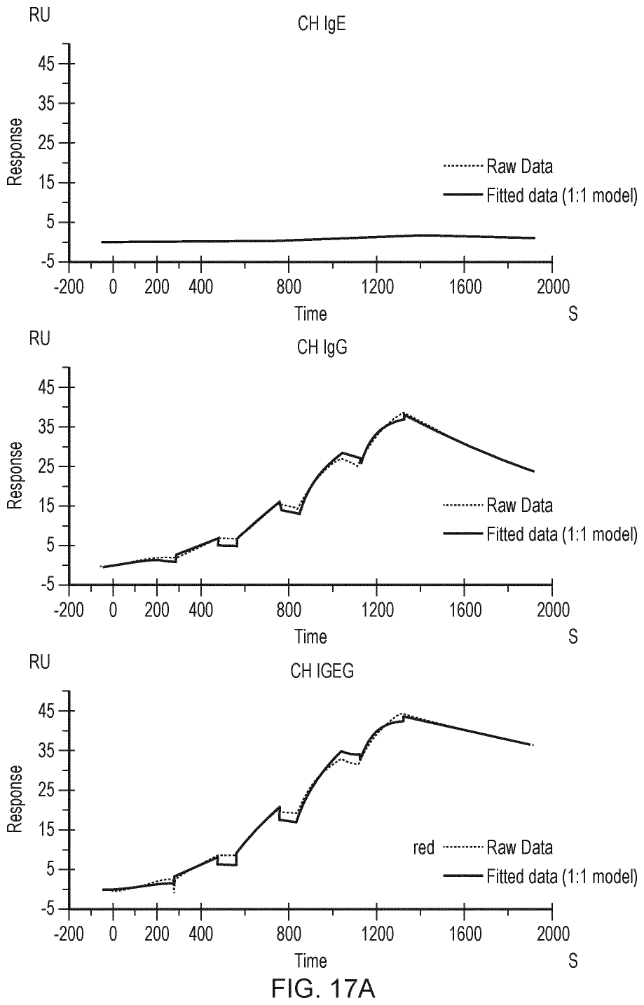

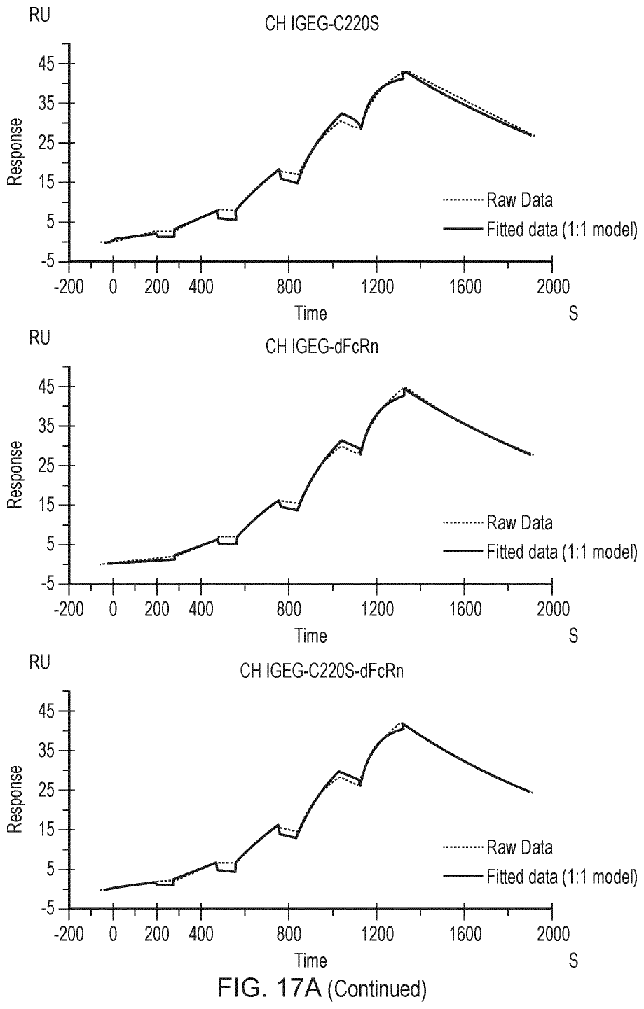

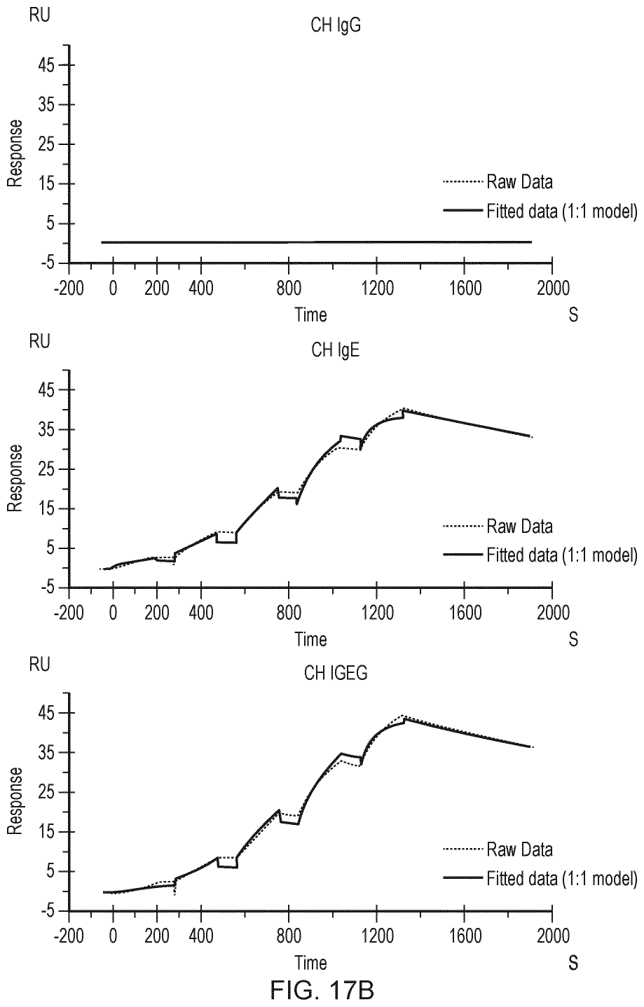

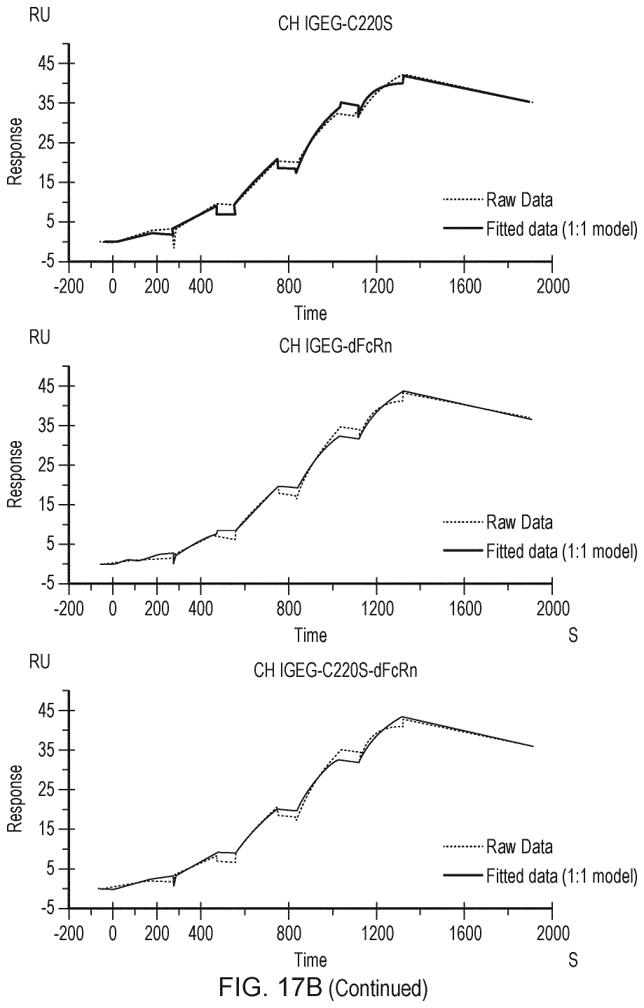

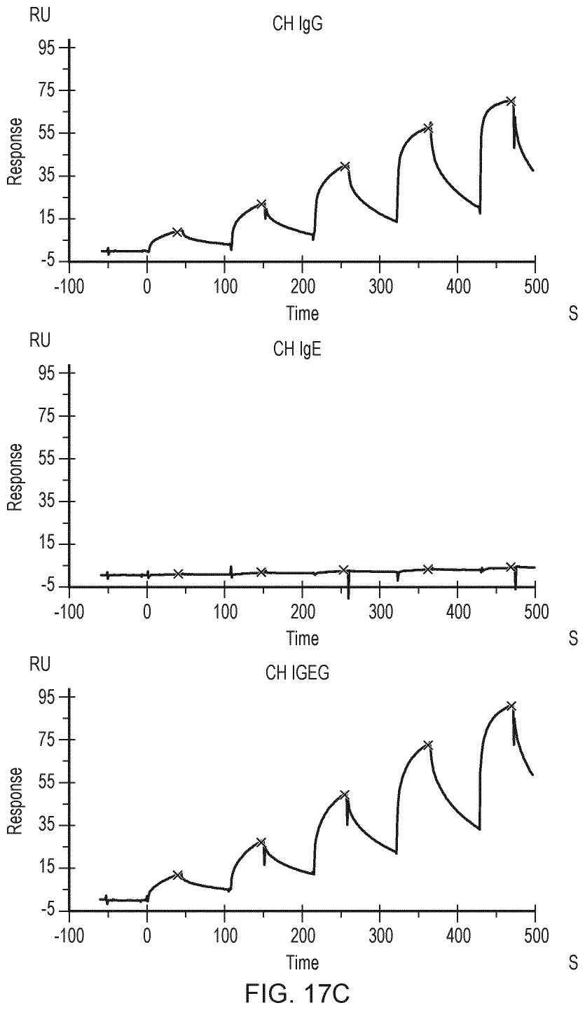

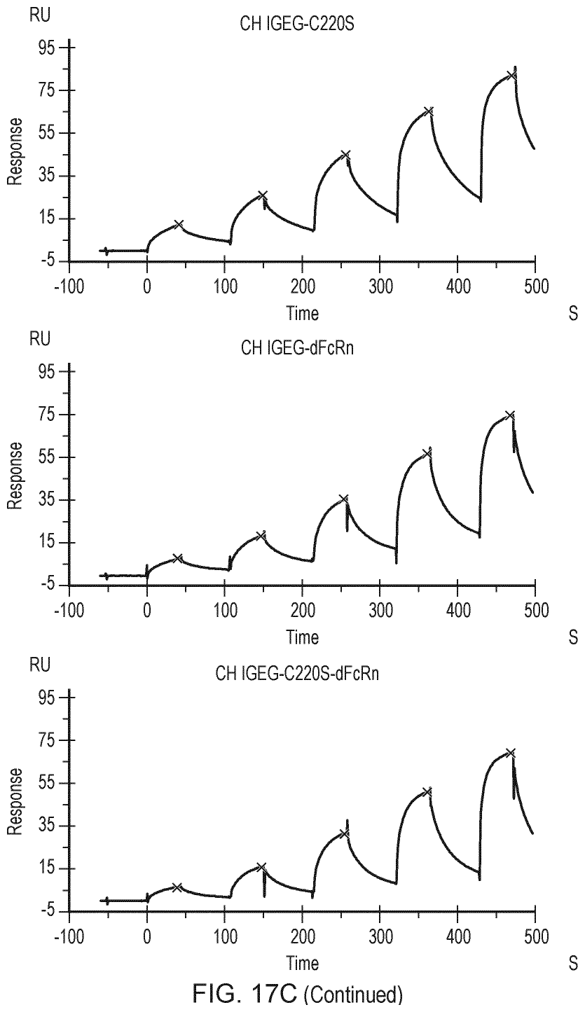

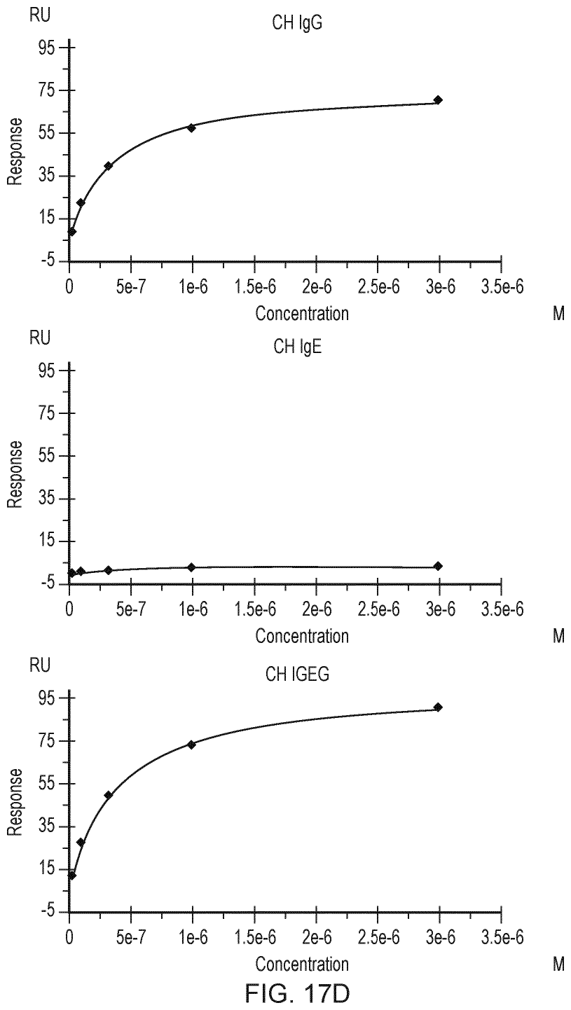

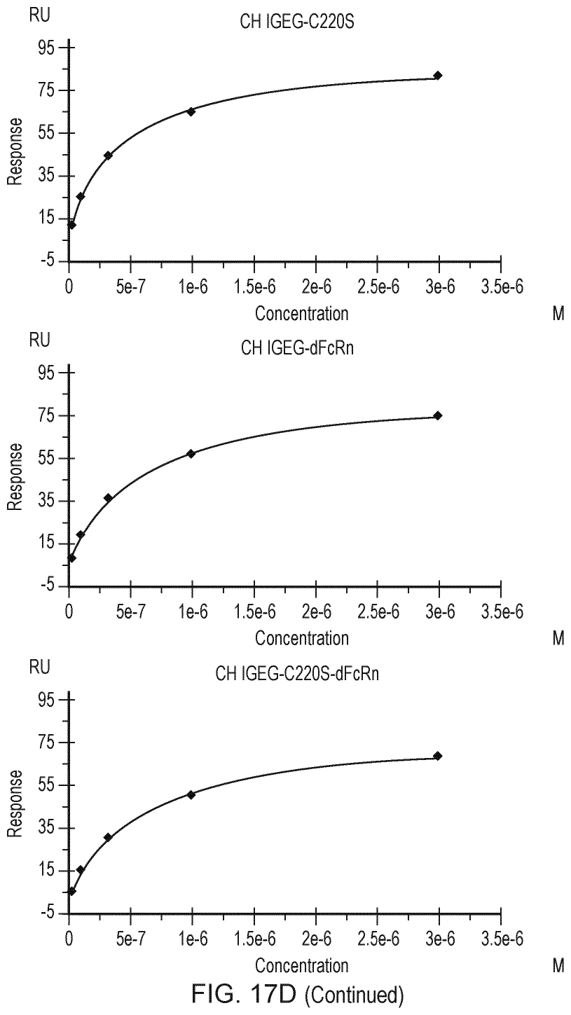

At the start of each cycle His-tagged Fc receptors diluted in HEPES buffered saline containing 0.05% v/v Surfactant P20 (HBS-P+) were loaded to a specified RU level (Table 7). A five point, three-fold dilution range of test antibody without regeneration between each concentration was used for each receptor tested. The target RU loaded for each Fc receptor, association and dissociation times used for test antibody binding together with the concentration range used for each test antibody are shown in (Table 7). In all cases, antibodies were passed over the chip in increasing concentrations followed by a single dissociation step. Following dissociation, the chip was regenerated with two injections of Glycine pH 1.5. The signal from the reference channel F_{c}1 (blank) was subtracted from that of the F_{c} loaded with receptor to correct for differences in non-specific binding to the reference surface. High affinity interactions were analysed using 1:1 fit (see FIGS. 17a and 17b for example data), whereas the low affinity interactions were analysed using a steady state model (see FIGS. 17c and 17d for example data). Table 8 shows a summary of the data obtained. IGEG variants bound to both the Fcgamma receptors tested and to Fcepsilon receptor. IgG control found to the Fcgamma receptors and not to Fcepsilon, whereas conversely, IgE control found to the Fcepsilon receptor and not to the Fcgamma receptors tested.

TABLE 7
Experimental parameters (as defined within the experimental setup) used for the
assessment of binding of IGEG variants to Fc gamma and Fc epsilon receptors using Biacore
single cycle kinetics.

| | Binding | RU | Concentration | | | |
|---|---|---|---|---|---|---|
| Name | affinity | loaded | Range (nM) | Association(s) | Dissociation(s) | Analysis |
| FcγRI | High | 30 | 0.411 to 33.33 | 200 | 600 | 1:1 |
| | | | | | | Affinity |
| FcγRIIIA_{176Phe} | Low | 20 | 98.8 to 8000 | 45 | 25 | Steady |
| | | | | | | State |
| FcγRIIIA_{176Val} | Low | 20 | 98.8 to 8000 | 45 | 25 | Steady |
| | | | | | | State |
| FcγRIIIB | Low | 60 | 98.8 to 8000 | 45 | 25 | Steady |
| | | | | | | State |
| Fcε Ria | High | 30 | 0.411 to 33.33 | 200 | 600 | 1:1 |
| | | | | | | Affinity |

TABLE 8
1:1 (FcgRI and FceRIa ) or Steady state affinity (FcγRIIIA_{176Phe}, FcγRIIIA_{176Val} and FcγRIIIB) summary data for the binding of
Trastuzumab and HMW-MAA-IGEG variants to Fc gamma and Fc epsilon receptors, as determined using Biacore single cycle kinetics.

| | | | Human CD16A | | Human CD16A | | | | | |
|---|---|---|---|---|---|---|---|---|---|---|
| | Human CD64 | | 176 Phe | | 176 Val | | Human CD16B | | | |
| | (FcgRI) | | (FCγRIIIA_{176Phe}) | | (FcγRIII_{176Val}) | | (FcγRIIIB) | | Human FceRIa | |
| | | Relative | | Relative | | Relative | | Relative | | Relative |
| Antibody | K_{D} (M) | binding | K_{D} (M) | Binding | K_{D} (M)* | Binding | K_{D} (M)* | Binding | K_{D} (M) | binding |
| Control IgG1 | 2.47E−09 | ++++ | 2.19E−06 | ++ | 7.20E−07 | +++ | 3.89E−06 | ++ | — | — |
| Trastuzumab_IgE | — | — | — | — | — | — | — | — | 4.11E−10 | +++++ |
| Trastuzumab_IGEG | 2.35E−09 | ++++ | 6.64E−07 | +++ | 2.20E−07 | +++ | 1.70E−06 | ++ | 5.38E−10 | +++++ |
| Trastuzumab_IGEG-C220S | 2.37E−09 | ++++ | 6.95E−07 | +++ | 2.33E−07 | +++ | 1.73E−06 | ++ | 5.57E−10 | +++++ |
| Trastuzumab_IGEG-dFcRn | 3.27E−09 | ++++ | 1.32E−06 | ++ | 4.38E−07 | +++ | 2.54E−06 | ++ | 5.65E−10 | +++++ |
| Trastuzumab_IGEG-C220S-dFcRn | 3.55E−09 | ++++ | 1.42E−06 | ++ | 4.88E−07 | +++ | 3.08E−06 | ++ | 5.77E−10 | +++++ |
| HMW-MAA_IgG | 2.65E−09 | ++++ | 8.42E−07 | +++ | 3.32E−07 | +++ | 2.02E−06 | ++ | — | — |
| HMW-MAA_IgE | — | — | — | — | — | — | — | — | 5.08E−10 | +++++ |
| HMW-MAA_IGEG | 1.63E−09 | ++++ | 6.65E−07 | +++ | 3.86E−07 | +++ | 1.48E−06 | ++ | 5.99E−10 | +++++ |
| HMW-MAA_IGEG-C220S | 1.67E−09 | ++++ | 7.75E−07 | +++ | 4.00E−07 | +++ | 1.87E−06 | ++ | 4.93E−10 | +++++ |
| HMW-MAA_IGEG-dFcRn | 2.02E−09 | ++++ | 9.60E−07 | +++ | 5.35E−07 | +++ | 2.03E−06 | ++ | 5.56E−10 | +++++ |
| HMW-MAA_IGEG-C220S-dFcRn | 2.18E−09 | ++++ | 1.16E−06 | +++ | 6.01E−07 | +++ | 2.78E−06 | ++ | 5.35E−10 | +++++ |

Assessment of IGEG Variant Binding to Human FcRn

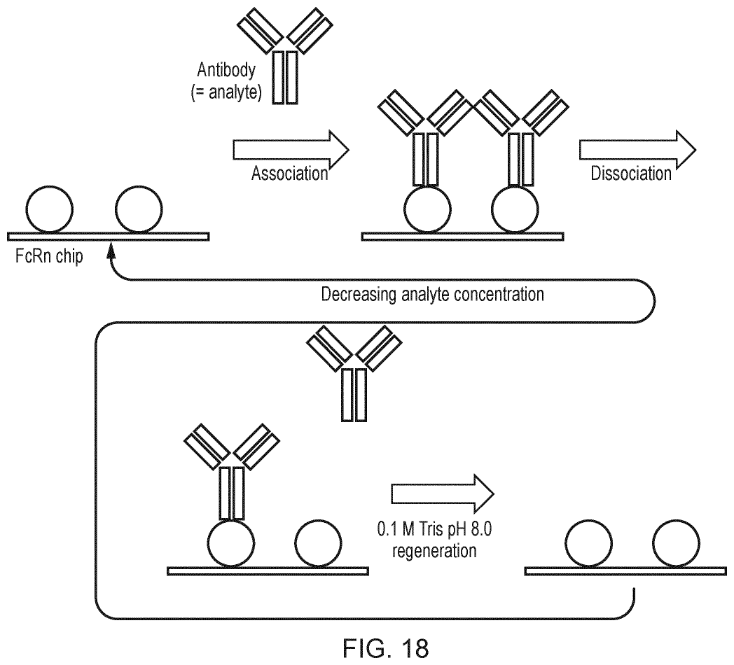

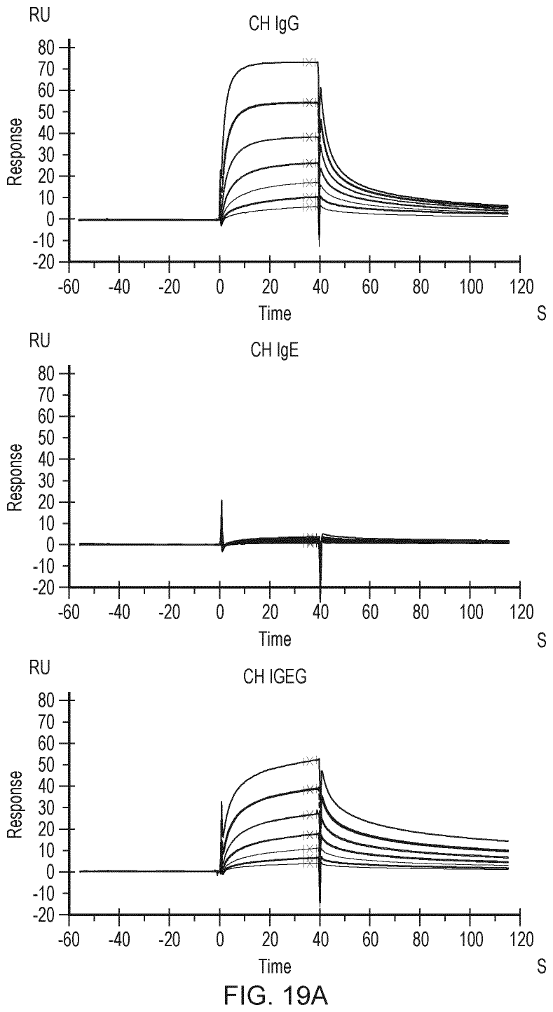

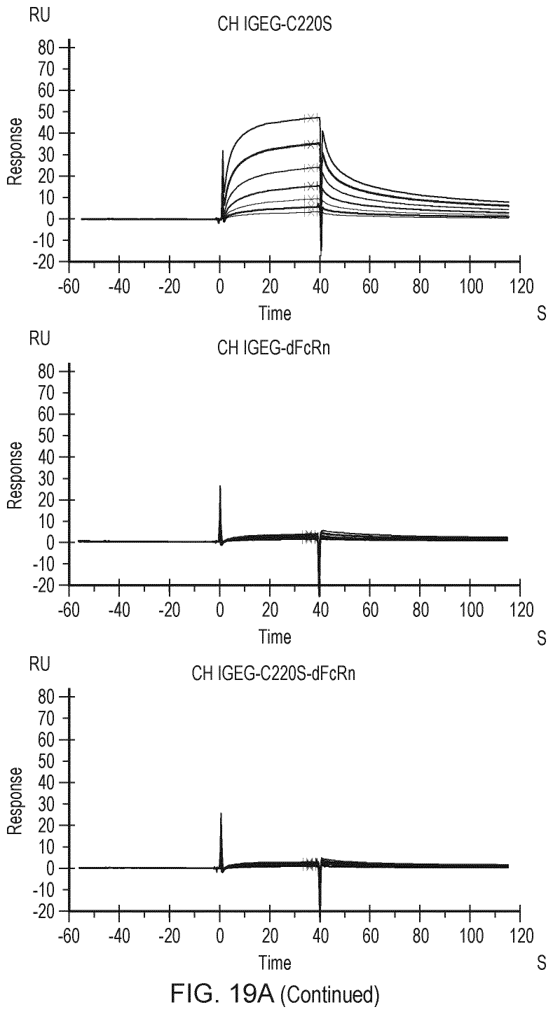

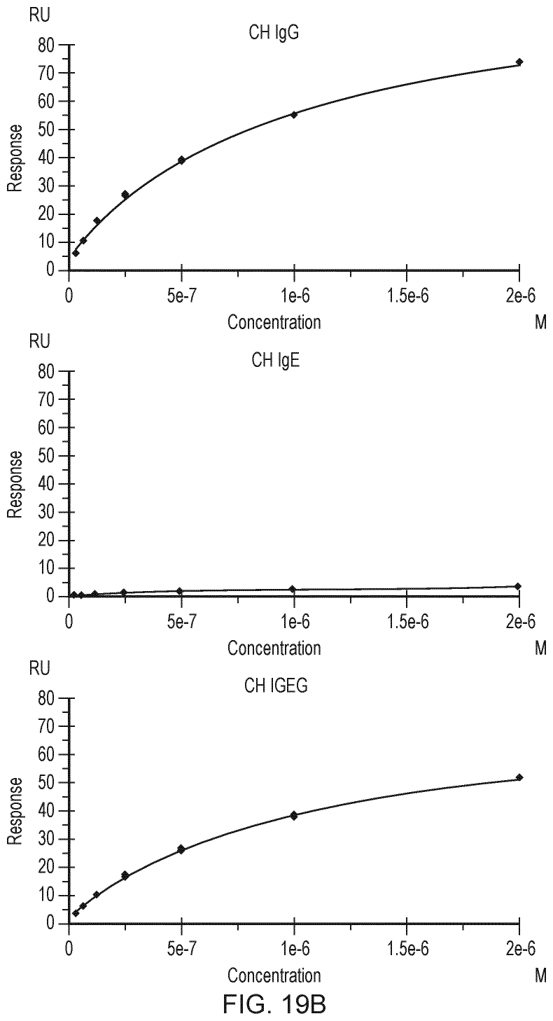

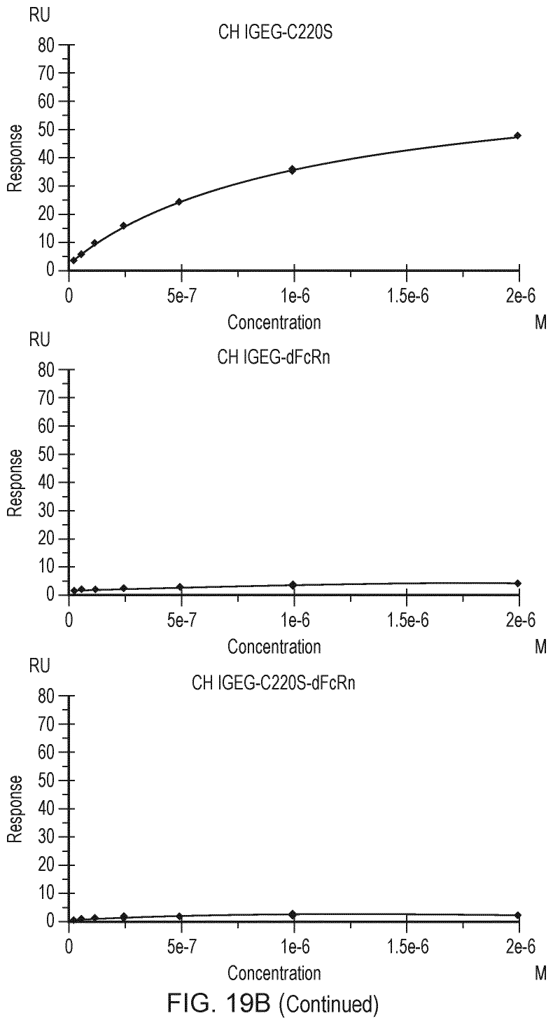

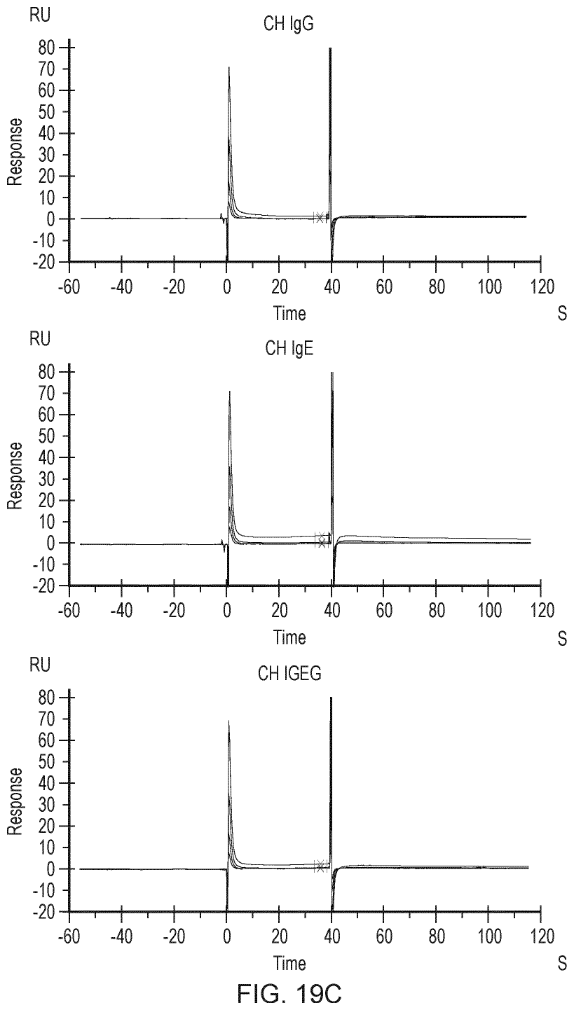

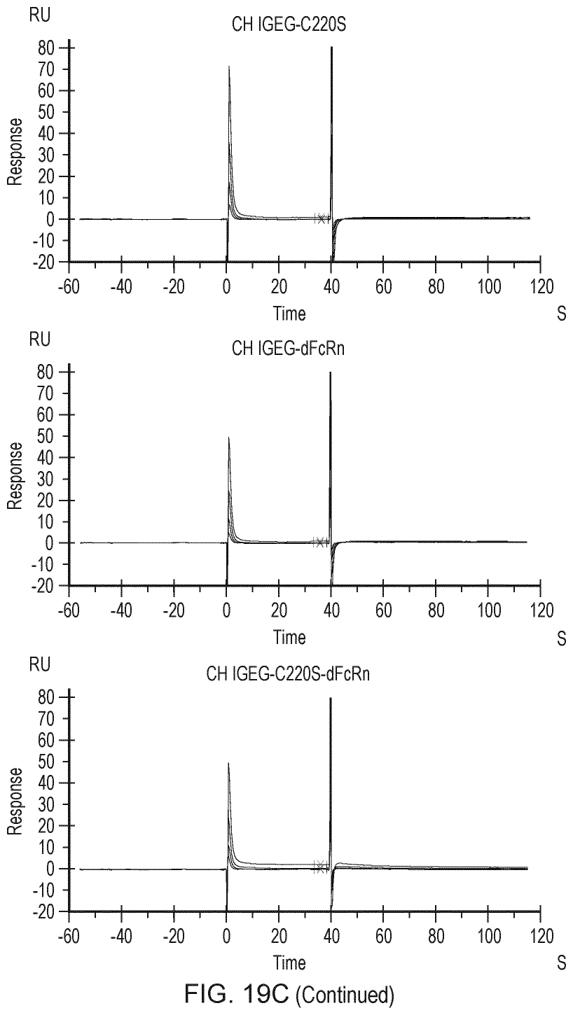

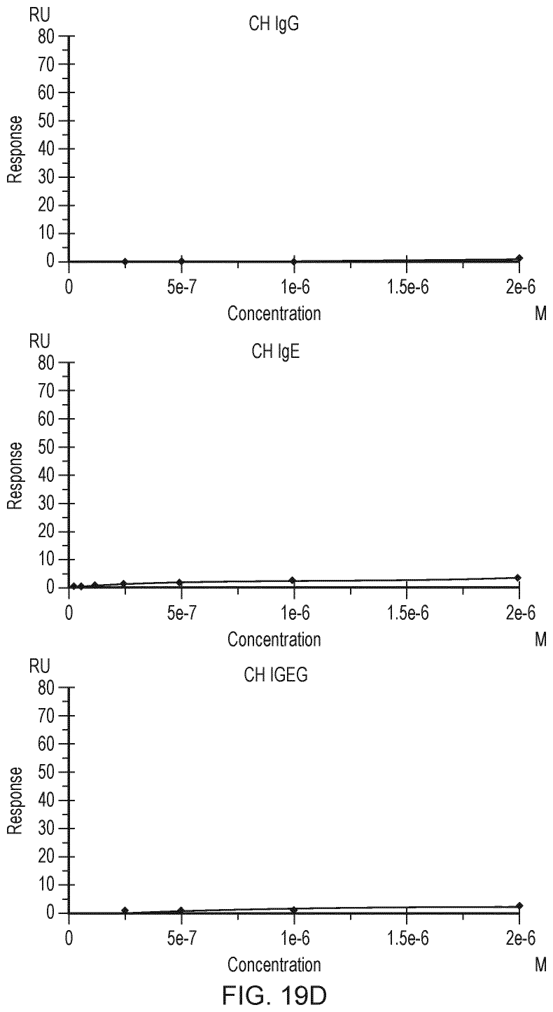

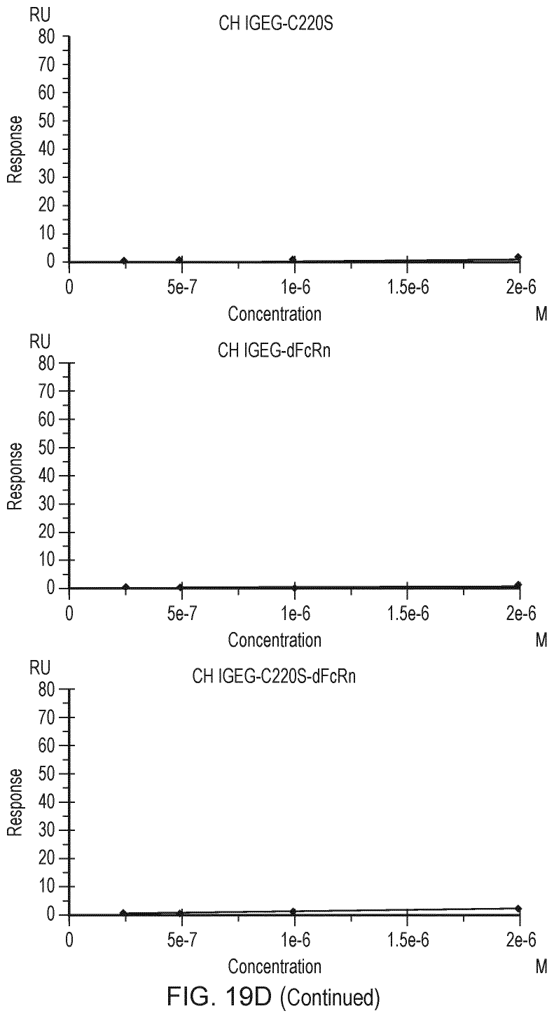

The binding of the purified antibodies to FcRn was assessed by steady state affinity analysis using a Biacore T200 (serial no. 1909913) instrument running Biacore T200 Evaluation Software V3.0.1 (Uppsala, Sweden). hFcRn (Sino Biological, Beijing, China) was coupled onto a Series S CM5 (carboxymethylated dextran) sensor chip (Cytiva, Uppsala, Sweden) at 10 μg/mL in sodium acetate pH 5.5 using standard amine coupling. Purified HMW-MAA antibodies were titrated in a seven point, two fold dilution from 31.25 nM to 2000 nM in PBS containing 0.05% Polysorbate 20 (P20) at pH 6.0 or a four three point, two-fold dilution from 250 nM to 2000 nM in PBS containing 0.05% Polysorbate 20 (P20) at pH 7.4. Antibodies were passed over the chip with increasing concentrations at a flow rate of 30 μl/min and at 25° C. The injection time was 40 s per concentration and the dissociation time was 75 s. Following a single dissociation, the chip was regenerated with 0.1 M Tris pH 8.0 FIG. 18 shows a schematic of the assay used to assess used to assess antibody binding to FcRn. Interactions were analysed using a steady state model (see FIGS. 19a to 19d for example data). Table 9 shows a summary of the data obtained. IGEG variants bound to FcRn at pH 6.0 with the exception of those in which the FcRn binding site has been removed (dFcRn) and which failed to bind FcRn. IgG control found to FcRn as expected whereas IgE did not show any binding to FcRn.

TABLE 9
Steady state affinity summary data for the binding of Trastuzumab
and HMW-MAA-IGEG variants to FcRn at pH 6.0 or pH 7.4,
as determined using Biacore single cycle kinetics.

| | FcRn pH 6.0 | FcRn pH 7.4 |
|---|---|---|
| Antibody | K_{D} (M) | K_{D} (M) |
| Control IgG1 | 6.12E−07 | — |
| Trastuzumab_IgE | — | — |
| Trastuzumab_IGEG | 4.77E−07 | — |
| Trastuzumab_IGEG-C220S | 5.06E−07 | — |
| Trastuzumab_IGEG-dFcRn | — | — |
| Trastuzumab_IGEG-C220S-dFcRn | — | — |
| HMW-MAA_IgG | 9.58E−07 | — |
| HMW-MAA_IgE | — | — |
| HMW-MAA_IGEG | 1.02E−06 | — |
| HMW-MAA_IGEG-C220S | 1.05E−06 | — |
| HMW-MAA_IGEG-dFcRn | — | — |
| HMW-MAA_IGEG-C220S-dFcRn | — | — |

UNcle Biostability Platform Analysis of IGEG Variants

IGEG variants were analysed for thermal stability using the UNcle biostability platform (Unchained labs, Pleasanton, USA). Thermal ramp stability experiments (Tm and Tagg) are well established methods for ranking proteins and formulations for stability. A protein's denaturation profile provides information about its thermal stability and represents a structural ‘fingerprint’ for assessing structural and formulation buffer modifications. A widely used measure of the thermal structural stability of a protein is the temperature at which it unfolds from the native state to a denatured state. For many proteins, this unfolding process occurs over a narrow temperature range and the mid-point of this transition is termed ‘melting temperature’ or ‘Tm’. To determine the melting temperature of a protein, UNcle measures the fluorescence of Sypro Orange (which binds to exposed hydrophobic regions of proteins) as the protein undergoes conformational changes.

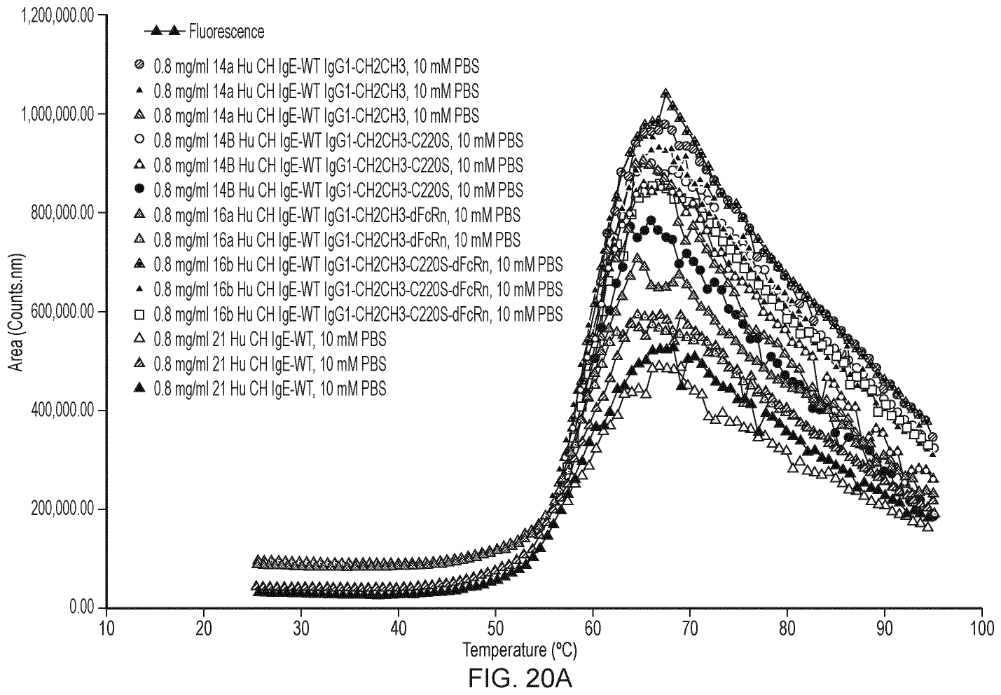

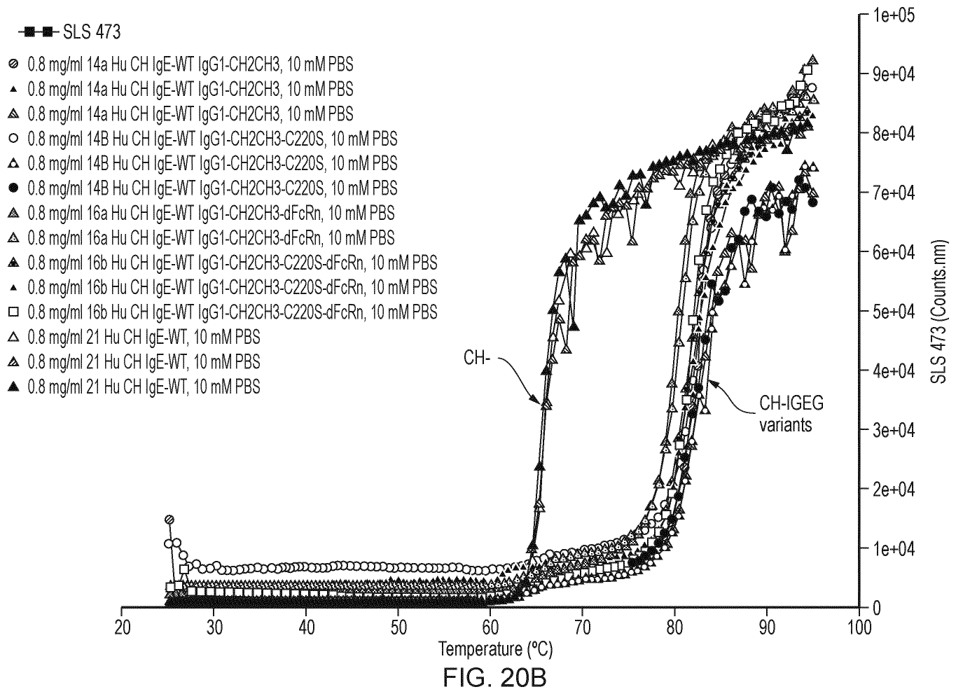

Samples for each variant were formulated in PBS and Sypro Orange at a final concentration of 0.8 mg/mL. 9 μL of each sample mixture was loaded in duplicate into UNi microcuvettes. Samples were subjected to a thermal ramp from 25-95° C., with a ramp rate of 0.3° C./minute and excitation at 473 nm. Full emission spectra were collected from 250-720 nm, and the area under the curve between 510-680 nm was used to calculate the inflection points of the transition curves (Tₒₙₛₑₜ and Tₘ). Monitoring of static light scattering (SLS) at 473 nm allowed the detection of protein aggregation, and T_{agg} (onset of aggregation) was calculated from the resulting SLS profiles. Data analysis was performed using UNcle™ software version 4.0 and summarised in Table 10. Tm1 values were broadly consistent within each set of variants and between IgE and IGEG variants (FIG. 20a), however, the IGEG variants showed a significant improvement in static light scattering profile compared to the equivalent IgE variants alone (FIG. 20b).

TABLE 10
Summary of thermal stability values for the purified IGEG
variants, as determined using the UNcle biostability platform.

| | | | T_{agg} |
|---|---|---|---|
| | Tₘ1 | Tₒₙₛₑₜ | (° C.) |
| | (° C.) | (° C.) | (473 nm) |
| Antibody | Average | Average | Average |
| Trastuzumab IGEG | 57.5 | 50.4 | 76.7 |
| Trastuzumab IGEG-C220S | 57.6 | 50.3 | 78.2 |
| Trastuzumab IGEG-dFcRn | 58.1 | 51.7 | 76.7 |
| Trastuzumab IGEG-C220S-dFcRn | 57.5 | 51.5 | ND |
| Trastuzumab IgE-WT | 56.6 | 45.7 | 66 |
| HMW-MAA IGEG | 59.4 | 52.0 | 77.4 |
| HMW-MAA IGEG-C220S | 59.1 | 51.3 | 77.6 |
| HMW-MAA IGEG-dFcRn | 58.9 | 50.0 | 75.7 |
| HMW-MAA IGEG-C220S-dFcRn | 59.5 | 51.4 | 76.7 |
| HMW-MAA IgE-WT | 57.2 | 48.2 | 63.5 |

### Example 7—Assessment of IGEG Variant Binding to A375 Cells

Binding of the HMW-MAA (CSPG4) antibody variants detailed in Example 6 to HMW-MAA was assessed using A375 cells HMW-MAA.

Method

Harvesting A375 Cells

A375 cells were cultured using standard methods. When A375 cells were confluent, the cells were harvested. In brief, cells were washed with PBS before incubation with TrypLE™ at 37° C. for 10 minutes to detach the cells from the flask. Cells were resuspended in 10 mL of media and centrifuged for 3 minutes at 250 g. Cells were then resuspended in 1 mL FACS buffer and counted on the Cellometer® to determine the cell number and viability. Following this, cells were diluted to 1×10⁶ cells per mL with FACS buffer, and 100 μL of this cell suspension plated per well on a plate.

Binding Assay

Binding of purified IGEGs to A375 cells (ATCC, Virginia, US) was assessed by flow cytometry using a Attune® NxT Acoustic Focusing Cytometer running Attune Software V3.1.2 (ThermoFisher Scientific, Loughborough, UK). A375 cells were incubated with the primary antibodies (as described in Example 6) for 30 min at 4° C. followed by incubation with FITC conjugated Goat anti-human anti-IgG or IgE secondary antibodies (Vector Laboratories, California, US) at 10 μg/ml for a further 30 minutes at 4° C. Cells were washed and resuspended in FACS buffer and then acquired on the Attune® NxT Acoustic Focusing Cytometer. The data was analysed using FlowJo™ Software Version 10 (Becton, Dickinson and Company, New Jersey, US) and GraphPad Prism 8 (GraphPad Software, California, US).

Results

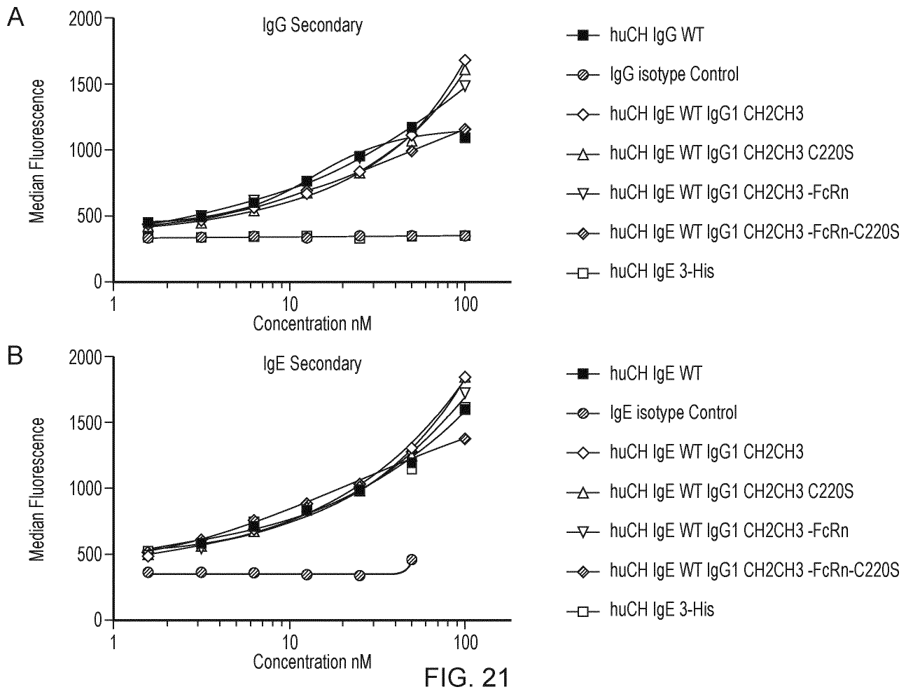

As demonstrated in FIGS. 21a and 21b, all HMW-MAA antibodies and variants bound to A375 cells.

### Example 8: ADCC and ADCP Assays

Assays were performed to determine the effects of the described antibodies on levels of both antibody-dependent cell-mediated phagocytosis (ADCP) and antibody dependent cell-mediated cytotoxicity (ADCC), the two main mechanisms by which immune effector cells can kill tumour cells. The trastuzumab antibody variants described in Example 6 were compared to Trastuzumab IgE and Herceptin IgG antibodies.

Method

ADCC and ADCP assays were performed using methods similar to those existing in the art (for example, see Three-colour flow cytometric method to measure antibody-dependent tumour cell killing by cytotoxicity and phagocytosis. J Immunol Methods. 2007 Jun. 30; 323(2):160-71) using U-937 effector cells and SK-BR-3 target cells.

The day prior to performing the assay, Her2-expressing tumour cells (SK-BR-3) were stained. To do this, SK-BR-3 cells were detached from the plate using TrypLE, washed with complete RPMI media (RPMI 1640 media supplemented with pen/strep and 10% HI FBS) before adding to serum-free HBSS. 0.75 μL 0.5 mM carboxyflourescein succinimidyl ester (CSFE) in HBSS was added per 1×10⁶ cells and cells incubated at 37° C. for 10 minutes. After washing, cells were plated and incubated overnight.

The next day, U-937 effector cells were passaged, counted using Trypan blue and resuspended in complete RPMI media to provide 1.5×10⁶ cells per mL. The CFSE-labelled SK-BR-3 cells were detached by TrypLE treatment, washed, counted, and re-suspended in complete RPMI media to provide 0.5×10⁶ cells per mL. The Trastuzumab IgE, Herceptin IgG, Trastuzumab-IGEG, Trastuzumab-IGEG-C220S, and IgG isotype antibodies detailed in Example 6 were then diluted to a starting concentration of 120 nM and then serially diluted by a factor of six. 25 μL of each antibody dilution was added to a 96-well plate in duplicate along with 50 μL of the SK-BR-3 cell suspension (equivalent to 25000 cells) and 25 μL of the U-937 effector cell suspension (equivalent to 37500 cells). Appropriate control wells lacking one or more of: CSFE staining, U-397 cells, SK-BR-3 cells, viable SK-BR-3 cells (replaced by heat-shocked SK-BR-3 cells) or test antibody were included in the assay. The plate was then incubated for 3 hours at 37° C., centrifuged and washed with FACS buffer (PBS+2% FCS) twice before resuspending in 100 μL FACS with 2 μL CD89 APC-conjugated labelling antibody. Control wells were resuspended in FACS buffer alone. After 30 minutes at 4° C., the plate was centrifuged and washed again with FACS buffer twice before resuspending the cells in 100 μL FACS buffer containing propidium iodide (PI) stain (5 μL per 100 μL). Control wells were resuspended in FACS buffer and incubated for 15 minutes at room temperature.

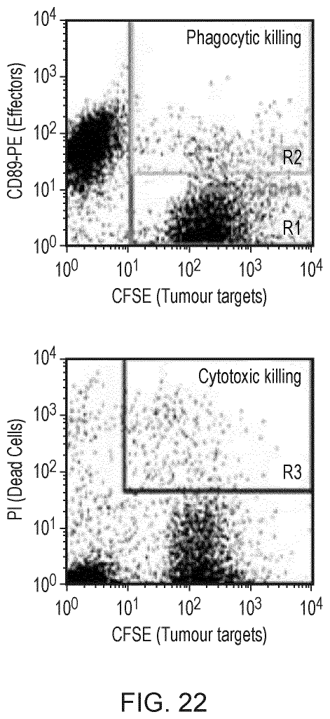

50,000 cells/tube were then acquired on the Attune™ NxT Acoustic Focusing Cytometer. Compensation was set-up using control wells. R1, R2, R3 gating was applied in analysis software (Flow Jo) (FIG. 22) and cell counts obtained per gate. Calculations were then performed to determine the cytotoxicity (ADCC) or phagocytic (ADCP) activity.

Results

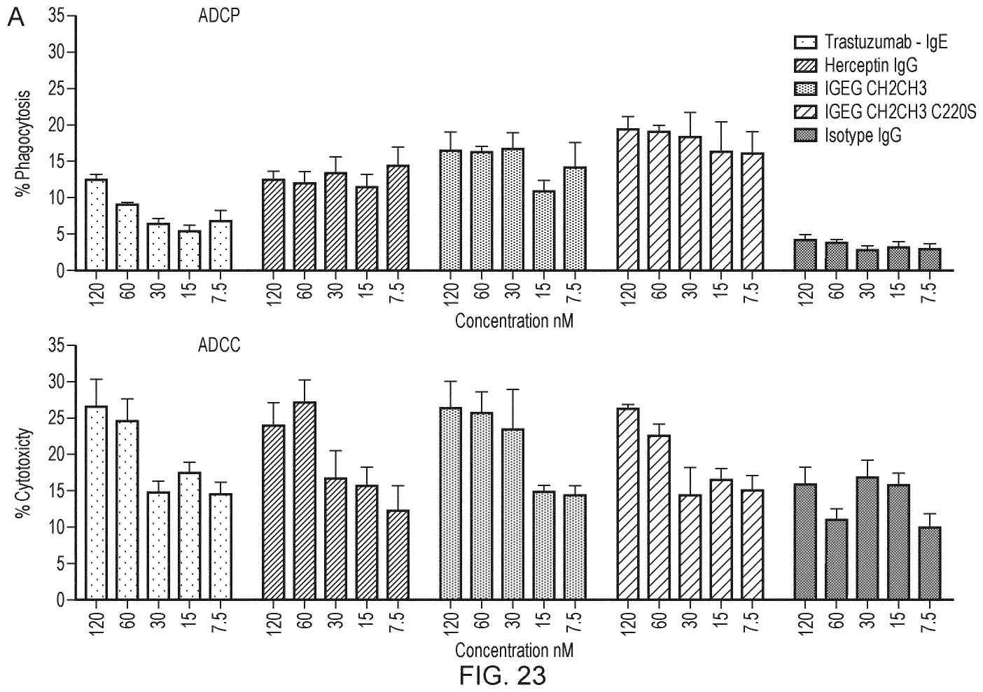

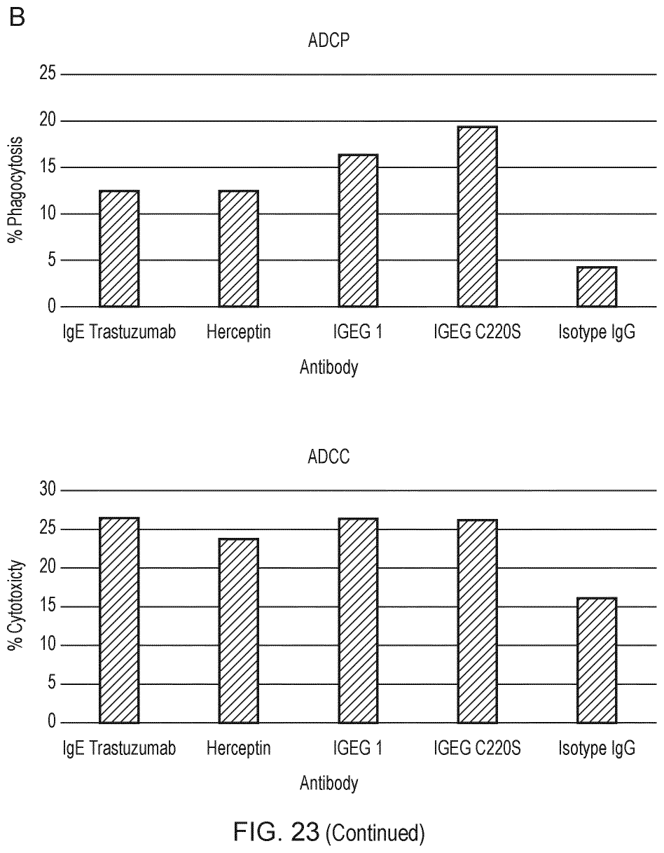

As demonstrated in FIG. 23, the Trastuzumab-IGEG (IGEG-CH2CH3) antibody appears to result in higher levels of phagocytosis than the Herceptin IgG and Trastuzumab IgE antibodies across all concentrations tested (120-7.5 nM). The Trastuzumab-IGEG-C200S (IGEG-CH2CH3-C220S) antibody appears to result in higher levels of phagocytosis than the Herceptin IgG and Trastuzumab IgE antibodies. In addition, the results demonstrate that the Trastuzumab IgE, Herceptin IgG and both IGEG antibodies had comparable effects on cytotoxicity.

The present application claims priority from UK patent application no. 1914165.4, filed 1 Oct. 2019, UK patent application no. 1917059.6, filed 22 Nov. 2019 and UK patent application no. 2008248.3, filed 2 Jun. 2020, the contents of which are incorporated herein by reference. All publications mentioned in the above specification are herein incorporated by reference. Various modifications and variations of the described embodiments of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1-43. (canceled)

44. A fusion protein that binds an Fcε receptor and an Fcγ receptor, said fusion protein comprising:
(i) one or more heavy chain constant domains, or variants or functional fragments thereof, derived from an Immunoglobulin E (IgE); and
(ii) one or more constant domains, or variants or functional fragments thereof, derived from an Immunoglobulin G (IgG).

45. The fusion protein of claim 44, comprising a Cε3 domain, or a variant or functional fragment thereof.

46. The fusion protein of claim 45, comprising a Cε2 domain, a Cε3 domain, and a Cε4 domain, or variants or functional fragments thereof.

47. The fusion protein of claim 44, comprising a Cγ2 domain, or a variant or functional fragment thereof.

48. The fusion protein of claim 47, further comprising a Cγ3 domain, or a variant or functional fragment thereof.

49. The fusion protein of claim 48, further comprising all or part of an IgG hinge region.

50. The fusion protein of claim 44, comprising a tetrameric IgE and at least one binding site for one or more Fcγ receptors.

51. The fusion protein of claim 50, wherein the at least one binding site for one or more Fcγ receptors is fused to the C terminus of an IgE heavy chain.

52. The fusion protein of claim 44, wherein the IgG is IgG1.

53. The fusion protein of claim 44, wherein said fusion protein binds to FcγRIIIa.

54. The fusion protein of claim 44, wherein said fusion protein binds to FcεRI.

55. The fusion protein of claim 44, comprising an amino acid sequence having at least 85%, 90%, 95%, or 99% sequence identity with a sequence selected from the group consisting of SEQ ID NOs:1 to 5.

56. The fusion protein of claim 44, comprising an amino acid sequence having at least 85%, 90%, 95%, or 99% sequence identity with a sequence selected from the group consisting of SEQ ID NOs: 9, 10, 11, 23, 24, 25, 26, 163-166, 169-172, 174, 175, and 176.

57. The fusion protein of claim 56, comprising:
(i) a variant of SEQ ID NO; 10 comprising the substitution(s) Ile23Ala and/or His80Ala;
(ii) a variant of SEQ ID NO; 11 comprising the substitution His95Ala; or
(iii) a variant of SEQ ID NO; 9 comprising the substitution Cys5Ser.

58. The fusion protein of claim 44, comprising:
(i) an IgE amino acid sequence having at least 85%, 90%, 95%, or 99% sequence identity with each of SEQ ID NOs: 3, 4, and/or 5; and
(ii) an IgG amino acid sequence having at least 85%, 90%, 95%, or 99% sequence identity with; (a) each of SEQ ID NOs; 9, 10, and/or 11; or (b) each of SEQ ID NOs: 174, 175, and 176.

59. The fusion protein of claim 58, wherein the IgG amino acid sequence is fused at the C terminus of the IgE amino acid sequence.

60. The fusion protein of claim 44, wherein said fusion protein binds specifically to a cancer antigen.

61. The fusion protein of claim 44, comprising all six of the sequences from one of the following groups of SEQ ID NOs: SEQ ID NOs: 27-32, SEQ ID NOs: 33-38, SEQ ID NOs: 39-45, SEQ ID NOs: 46-51, SEQ ID NOs: 52-57, SEQ ID NOs: 58-63, SEQ ID NOs: 64-69, SEQ ID NOs: 70-75, SEQ ID NOs: 76-81, SEQ ID NOs: 82-87, SEQ ID NOs: 88-93, SEQ ID NOs: 94-99, or SEQ ID NOs: 100-105.

62. The fusion protein of claim 44, comprising a variable domain with an amino acid sequence having at least 85%, 90%, 95%, or 99% sequence identity with a sequence selected from the group consisting of SEQ ID NO: 1, 160, 161, 162, 177, and 178.

63. The fusion protein of claim 44, wherein said fusion protein binds to FcγRI and/or FcγRIIIb.

64. The fusion protein of claim 44, wherein said fusion protein comprises a modified IgG hinge region lacking a free cysteine residue.

65. The fusion protein of claim 44, wherein said fusion protein shows increased thermal stability compared to an IgE.

66. The fusion protein of claim 44, wherein said fusion protein does not bind to FcRn.

67. The fusion protein of claim 44, wherein said fusion protein comprises a modified IgG CH2 and/or CH3 domain lacking one or more isoleucine or histidine residues associated with FcRn binding.

68. A pharmaceutical composition comprising a fusion protein of claim 44 and a pharmaceutically acceptable excipient, diluent, or carrier.


SEQUENCE LISTING (179 sequences)

SEQ ID NO: 1 - protein, 120 aa, Artificial Sequence [sequence omitted]

SEQ ID NO: 2 - protein, 103 aa, Artificial Sequence
  WT IgE_CH1
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFS

SEQ ID NO: 3 - protein, 107 aa, Artificial Sequence
  WT IgE_CH2
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 4 - protein, 108 aa, Artificial Sequence
  WT IgE_CH3
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 5 - protein, 110 aa, Artificial Sequence
  WT IgE_CH4
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 6 - protein, 7 aa, Artificial Sequence
  IgE Loop 1
LAPSKGT

SEQ ID NO: 7 - protein, 6 aa, Artificial Sequence
  IgE Loop 2
RNGTLT

SEQ ID NO: 8 - protein, 7 aa, Artificial Sequence
  IgE Loop 3
HPHLPRA

SEQ ID NO: 9 - protein, 15 aa, Artificial Sequence
  Wild type IgG hinge region
EPKSCDKTHTCPPCP

SEQ ID NO: 10 - protein, 110 aa, Artificial Sequence
  Wild type IgG CH2 domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 11 - protein, 107 aa, Artificial Sequence
  Wild type IgG CH3 domain
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 7 aa, Artificial Sequence
  IgG Fc?R-binding Loop 1
VSHEDPE

SEQ ID NO: 13 - protein, 6 aa, Artificial Sequence
  IgG Fc?R-binding Loop 2
YNSTYR

SEQ ID NO: 14 - protein, 7 aa, Artificial Sequence
  IgG Fc?R-binding Loop 3
NKALPAP

SEQ ID NO: 15 - protein, 108 aa, Artificial Sequence
  IgE_CH3 containing IgG FcgammaR-binding Loop 1
DSNPRGVSAYLSRPSPFDLFIRKSPTITCLVVDVSHEDPEVNLTWSRASGKPVNHSTRKEEKQRNGTLTVTSTLPVGTRDWIEGETYQCRVTHPHLPRALMRSTTKTS

SEQ ID NO: 16 - protein, 108 aa, Artificial Sequence
  IgE_CH3 containing IgG FcgammaR-binding Loop 2
DSNPRGVSAYLSRPSPFDLFIRKSPTITCLVVDLAPSKGTVNLTWSRASGKPVNHSTRKEEKQYNSTYRVTSTLPVGTRDWIEGETYQCRVTHPHLPRALMRSTTKTS

SEQ ID NO: 17 - protein, 108 aa, Artificial Sequence
  IgE_CH3 containing IgG FcgammaR-binding Loop 3
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTNKALPAPLMRSTTKTS

SEQ ID NO: 18 - protein, 108 aa, Artificial Sequence
  IgE_CH3 containing IgG Fc?R-binding Loop 1 + Loop 2
DSNPRGVSAYLSRPSPFDLFIRKSPTITCLVVDVSHEDPEVNLTWSRASGKPVNHSTRKEEKQYNSTYRVTSTLPVGTRDWIEGETYQCRVTHPHLPRALMRSTTKTS

SEQ ID NO: 19 - protein, 108 aa, Artificial Sequence
  IgE_CH3 containing IgG Fc?R-binding Loop 1 + Loop 3
DSNPRGVSAYLSRPSPFDLFIRKSPTITCLVVDVSHEDPEVNLTWSRASGKPVNHSTRKEEKQRNGTLTVTSTLPVGTRDWIEGETYQCRVTNKALPAPLMRSTTKTS

SEQ ID NO: 20 - protein, 108 aa, Artificial Sequence
  IgE_CH3 containing IgG Fc?R-binding Loop 2 + Loop 3
DSNPRGVSAYLSRPSPFDLFIRKSPTITCLVVDLAPSKGTVNLTWSRASGKPVNHSTRKEEKQYNSTYRVTSTLPVGTRDWIEGETYQCRVTNKALPAPLMRSTTKTS

SEQ ID NO: 21 - protein, 108 aa, Artificial Sequence; identical to SEQ ID NO: 20
  IgE_CH3 containing IgG Fc?R-binding Loop 2 + Loop 3

SEQ ID NO: 22 - protein, 108 aa, Artificial Sequence
  IgE_CH3 containing IgG Fc?R Loop 1 + Loop 2 + Loop 3
DSNPRGVSAYLSRPSPFDLFIRKSPTITCLVVDVSHEDPEVNLTWSRASGKPVNHSTRKEEKQYNSTYRVTSTLPVGTRDWIEGETYQCRVTNKALPAPLMRSTTKTS

SEQ ID NO: 23 - protein, 237 aa, Artificial Sequence
  IgE_CH4 plus IgG1 Hinge-CH2 (containing RS linker)
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 24 - protein, 344 aa, Artificial Sequence
  IgE_CH4 plus IgG1 Hinge-CH2-CH3 (containing RS linker)
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 675 aa, Artificial Sequence
  heavy chain of the IgE plus IgG1 Hinge-CH2 construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 26 - protein, 782 aa, Artificial Sequence
  Heavy chain of the IgE plus IgG1 Hinge-CH2-CH3 construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 8 aa, Artificial Sequence
  Alemtuzumab CDR H1
GFTFTDFY

SEQ ID NO: 28 - protein, 10 aa, Artificial Sequence
  Alemtuzumab CDR H2
IRDKAKGYTT

SEQ ID NO: 29 - protein, 12 aa, Artificial Sequence
  Alemtuzumab CDR H3
AREGHTAAPFDY

SEQ ID NO: 30 - protein, 6 aa, Artificial Sequence
  Alemtuzumab CDR L1
QNIDKY

SEQ ID NO: 31 - protein, 3 aa, Artificial Sequence
  Alemtuzumab CDR L2
NTN

SEQ ID NO: 32 - protein, 9 aa, Artificial Sequence
  Alemtuzumab CDR L3
LQHISRPRT

SEQ ID NO: 33 - protein, 5 aa, Artificial Sequence
  Atezolizumab CDR H1
DSWIH

SEQ ID NO: 34 - protein, 10 aa, Artificial Sequence
  Atezolizumab CDR H2
WISPYGGSTY

SEQ ID NO: 35 - protein, 7 aa, Artificial Sequence
  Atezolizumab CDR H3
RHWPGGF

SEQ ID NO: 36 - protein, 7 aa, Artificial Sequence
  Atezolizumab CDR L1
DVSTAVA

SEQ ID NO: 37 - protein, 6 aa, Artificial Sequence
  Atezolizumab CDR L2
SASFLY

SEQ ID NO: 38 - protein, 9 aa, Artificial Sequence
  Atezolizumab CDR L3
QQYLYHPAT

SEQ ID NO: 39 - protein, 5 aa, Artificial Sequence
  Avelumab CDR H1
SYIMM

SEQ ID NO: 40 - protein, 10 aa, Artificial Sequence
  Avelumab CDR H2
SIYPSGGITF

SEQ ID NO: 41 - protein, 9 aa, Artificial Sequence
  Avelumab CDR H3
IKLFTVTTV

SEQ ID NO: 42 - protein, 8 aa, Artificial Sequence
  Avelumab CDR L1
VGGYNYVS

SEQ ID NO: 43 - protein, 6 aa, Artificial Sequence
  Avelumab CDR L2
DVSNRP

SEQ ID NO: 44 - protein, 6 aa, Artificial Sequence
  Avelumab CDR L2
DVSNRP

SEQ ID NO: 45 - protein, 10 aa, Artificial Sequence
  Avelumab CDR L3
SSYTSSSTRV

SEQ ID NO: 46 - protein, 8 aa, Artificial Sequence
  Bevacizumab CDR H1
GYTFTNYG

SEQ ID NO: 47 - protein, 8 aa, Artificial Sequence
  Bevacizumab CDR H2
INTYTGEP

SEQ ID NO: 48 - protein, 16 aa, Artificial Sequence
  Bevacizumab CDR H3
AKYPHYYGSSHWYFDV

SEQ ID NO: 49 - protein, 6 aa, Artificial Sequence
  Bevacizumab CDR L1
QDISNY

SEQ ID NO: 50 - protein, 3 aa, Artificial Sequence
  Bevacizumab CDR L2
FTS

SEQ ID NO: 51 - protein, 9 aa, Artificial Sequence
  Bevacizumab CDR L3
QQYSTVPWT

SEQ ID NO: 52 - protein, 10 aa, Artificial Sequence
  Certolizumab CDR H1
GYVFTDYGMN

SEQ ID NO: 53 - protein, 18 aa, Artificial Sequence
  Certolizumab CDR H2
GWINTYIGEPIYADSVKG

SEQ ID NO: 54 - protein, 11 aa, Artificial Sequence
  Certolizumab CDR H3
ARGYRSYAMDY

SEQ ID NO: 55 - protein, 11 aa, Artificial Sequence
  Certolizumab CDR L1
KASQNVGTNVA

SEQ ID NO: 56 - protein, 6 aa, Artificial Sequence
  Certolizumab CDR L2
SASFLY

SEQ ID NO: 57 - protein, 8 aa, Artificial Sequence
  Certolizumab CDR L3
QQYNIYPL

SEQ ID NO: 58 - protein, 8 aa, Artificial Sequence
  Cetuximab CDR H1
GFSLTNYG

SEQ ID NO: 59 - protein, 7 aa, Artificial Sequence
  Cetuximab CDR H2
IWSGGNT

SEQ ID NO: 60 - protein, 13 aa, Artificial Sequence
  Cetuximab CDR H3
ARALTYYDYEFAY

SEQ ID NO: 61 - protein, 6 aa, Artificial Sequence
  Cetuximab CDR L1
QSIGTN

SEQ ID NO: 62 - protein, 3 aa, Artificial Sequence
  Cetuximab CDR L2
YAS

SEQ ID NO: 63 - protein, 9 aa, Artificial Sequence
  Cetuximab CDR L3
QQNNNWPTT

SEQ ID NO: 64 - protein, 5 aa, Artificial Sequence
  Durvalumab CDR H1
RYWMS

SEQ ID NO: 65 - protein, 10 aa, Artificial Sequence
  Durvalumab CDR H2
NIKQDGSEKY

SEQ ID NO: 66 - protein, 10 aa, Artificial Sequence
  Durvalumab CDR H3
EGGWFGELAF

SEQ ID NO: 67 - protein, 8 aa, Artificial Sequence
  Durvalumab CDR L1
RVSSSYLA

SEQ ID NO: 68 - protein, 6 aa, Artificial Sequence
  Durvalumab CDR L2
DASSRA

SEQ ID NO: 69 - protein, 9 aa, Artificial Sequence
  Durvalumab CDR L3
QQYGSLPWT

SEQ ID NO: 70 - protein, 10 aa, Artificial Sequence
  Efalizumab CDR H1
GYSFTGHWMN

SEQ ID NO: 71 - protein, 20 aa, Artificial Sequence
  Efalizumab CDR H2
GIMIHPSDSETRYNQKFKDI

SEQ ID NO: 72 - protein, 16 aa, Artificial Sequence
  Efalizumab CDR H3
ARIGIYFYGTTYFDYI

SEQ ID NO: 73 - protein, 11 aa, Artificial Sequence
  Efalizumab CDR L1
RASKTISKYLA

SEQ ID NO: 74 - protein, 6 aa, Artificial Sequence
  Efalizumab CDR L2
SGSTLQ

SEQ ID NO: 75 - protein, 8 aa, Artificial Sequence
  Efalizumab CDR L3
QQHNEYPL

SEQ ID NO: 76 - protein, 11 aa, Artificial Sequence
  Omalizumab CDR H1
GYSITSGYSWN

SEQ ID NO: 77 - protein, 17 aa, Artificial Sequence
  Omalizumab CDR H2
ASITYDGSTNYADSVKG

SEQ ID NO: 78 - protein, 14 aa, Artificial Sequence
  Omalizumab CDR H3
ARGSHYFGHWHFAV

SEQ ID NO: 79 - protein, 15 aa, Artificial Sequence
  Omalizumab CDR L1
RASQSVDYDGDSYMN

SEQ ID NO: 80 - protein, 6 aa, Artificial Sequence
  Omalizumab CDR L2
AASYLE

SEQ ID NO: 81 - protein, 8 aa, Artificial Sequence
  Omalizumab CDR L3
QQSHEDPY

SEQ ID NO: 82 - protein, 10 aa, Artificial Sequence
  Panitumumab CDR H1
GGSVSSGDYY

SEQ ID NO: 83 - protein, 7 aa, Artificial Sequence
  Panitumumab CDR H2
IYYSGNT

SEQ ID NO: 84 - protein, 11 aa, Artificial Sequence
  Panitumumab CDR H3
VRDRVTGAFDI

SEQ ID NO: 85 - protein, 6 aa, Artificial Sequence
  Panitumumab CDR L1
QDISNY

SEQ ID NO: 86 - protein, 3 aa, Artificial Sequence
  Panitumumab CDR L2
DAS

SEQ ID NO: 87 - protein, 9 aa, Artificial Sequence
  Panitumumab CDR L3
QHFDHLPLA

SEQ ID NO: 88 - protein, 8 aa, Artificial Sequence
  Pertuzumab CDR L1
GFTFTDYT

SEQ ID NO: 89 - protein, 8 aa, Artificial Sequence
  Pertuzumab CDR H2
VNPNSGGS

SEQ ID NO: 90 - protein, 12 aa, Artificial Sequence
  Pertuzumab CDR H3
ARNLGPSFYFDY

SEQ ID NO: 91 - protein, 6 aa, Artificial Sequence
  Pertuzumab CDR L1
QDVSIG

SEQ ID NO: 92 - protein, 3 aa, Artificial Sequence
  Pertuzumab CDR L2
SAS

SEQ ID NO: 93 - protein, 9 aa, Artificial Sequence
  Pertuzumab CDR L3
QQYYIYPYT

SEQ ID NO: 94 - protein, 8 aa, Artificial Sequence
  Rituximab CDR H1
GYTFTSYN

SEQ ID NO: 95 - protein, 8 aa, Artificial Sequence
  Rituximab CDR H2
IYPGNGDT

SEQ ID NO: 96 - protein, 13 aa, Artificial Sequence
  Rituximab CDR H3
ARSTYYGGDWFNV

SEQ ID NO: 97 - protein, 5 aa, Artificial Sequence
  Rituximab CDR L1
SSVSY

SEQ ID NO: 98 - protein, 3 aa, Artificial Sequence
  Rituximab CDR L2
ATS

SEQ ID NO: 99 - protein, 9 aa, Artificial Sequence
  Rituximab CDR L3
QQWTSNPPT

SEQ ID NO: 100 - protein, 8 aa, Artificial Sequence
  Trastuzumab CDR H1
GFNIKDTY

SEQ ID NO: 101 - protein, 8 aa, Artificial Sequence
  Trastuzumab CDR H2
IYPTNGYT

SEQ ID NO: 102 - protein, 13 aa, Artificial Sequence
  Trastuzumab CDR H3
SRWGGDGFYAMDY

SEQ ID NO: 103 - protein, 6 aa, Artificial Sequence
  Trastuzumab CDR L1
QDVNTA

SEQ ID NO: 104 - protein, 3 aa, Artificial Sequence
  Trastuzumab CDR L2
SAS

SEQ ID NO: 105 - protein, 9 aa, Artificial Sequence
  L3
QQHYTTPPT

SEQ ID NO: 106 - protein, 5 aa, Artificial Sequence
  Adalimumab CDR H1
DYAMH

SEQ ID NO: 107 - protein, 17 aa, Artificial Sequence
  Adalimumab CDR H2
AITWNSGHIDYADSVEG

SEQ ID NO: 108 - protein, 12 aa, Artificial Sequence
  Adalimumab CDR H3
VSYLSTASSLDY

SEQ ID NO: 109 - protein, 11 aa, Artificial Sequence
  Adalimumab CDR L1
RASQGIRNYLA

SEQ ID NO: 110 - protein, 7 aa, Artificial Sequence
  Adalimumab CDR L2
AASTLQS

SEQ ID NO: 111 - protein, 9 aa, Artificial Sequence
  Adalimumab CDR L3
QRYNRAPYT

SEQ ID NO: 112 - protein, 10 aa, Artificial Sequence
  Basiliximab CDR H1
GYSFTRYWMH

SEQ ID NO: 113 - protein, 17 aa, Artificial Sequence
  Basiliximab CDR H2
AIYPGNSDTSYNQKFEG

SEQ ID NO: 114 - protein, 8 aa, Artificial Sequence
  Basiliximab CDR H3
DYGYYFDF

SEQ ID NO: 115 - protein, 10 aa, Artificial Sequence
  Basiliximab CDR L1
SASSSRSYMQ

SEQ ID NO: 116 - protein, 7 aa, Artificial Sequence
  Basiliximab CDR L2
DTSKLAS

SEQ ID NO: 117 - protein, 7 aa, Artificial Sequence
  Basiliximab CDR L3
HQRSSYT

SEQ ID NO: 118 - protein, 10 aa, Artificial Sequence
  Belimumab CDR H1
GGTFNNNAIN

SEQ ID NO: 119 - protein, 17 aa, Artificial Sequence
  Belimumab CDR H2
GIIPMFGTAKYSQNFQG

SEQ ID NO: 120 - protein, 14 aa, Artificial Sequence
  Belimumab CDR H3
SRDLLLFPHHALSP

SEQ ID NO: 121 - protein, 11 aa, Artificial Sequence
  Belimumab CDR L1
QGDSLRSYYAS

SEQ ID NO: 122 - protein, 7 aa, Artificial Sequence
  Belimumab CDR L2
GKNNRPS

SEQ ID NO: 123 - protein, 11 aa, Artificial Sequence
  Belimumab CDR L3
SSRDSSGNHWV

SEQ ID NO: 124 - protein, 10 aa, Artificial Sequence
  Daclizumab CDR H1
GYTFTSYRMH

SEQ ID NO: 125 - protein, 17 aa, Artificial Sequence
  Daclizumab CDR H2
YINPSTGYTEYNQKFKD

SEQ ID NO: 126 - protein, 7 aa, Artificial Sequence
  Daclizumab CDR H3
GGGVFDY

SEQ ID NO: 127 - protein, 10 aa, Artificial Sequence
  Daclizumab CDR L1
SASSSISYMH

SEQ ID NO: 128 - protein, 7 aa, Artificial Sequence
  Daclizumab CDR L2
TTSNLAS

SEQ ID NO: 129 - protein, 9 aa, Artificial Sequence
  Daclizumab CDR L3
HQRSTYPLT

SEQ ID NO: 130 - protein, 6 aa, Artificial Sequence
  Infliximab CDR H1
IFSNHW

SEQ ID NO: 131 - protein, 10 aa, Artificial Sequence
  Infliximab CDR H2
RSKSINSATH

SEQ ID NO: 132 - protein, 7 aa, Artificial Sequence
  Infliximab CDR H3
NYYGSTY

SEQ ID NO: 133 - protein, 7 aa, Artificial Sequence
  Infliximab CDR L1
FVGSSIH

SEQ ID NO: 134 - protein, 7 aa, Artificial Sequence
  Infliximab CDR L2
KYASESM

SEQ ID NO: 135 - protein, 5 aa, Artificial Sequence
  Infliximab CDR L3
QSHSW

SEQ ID NO: 136 - protein, 10 aa, Artificial Sequence
  Natalizumab CDR H1
GFNIKDTYIH

SEQ ID NO: 137 - protein, 17 aa, Artificial Sequence
  Natalizumab CDR H2
RIDPANGYTKYDPKFQG

SEQ ID NO: 138 - protein, 14 aa, Artificial Sequence
  Natalizumab CDR H3
EGYYGNYGVYAMDY

SEQ ID NO: 139 - protein, 11 aa, Artificial Sequence
  Natalizumab CDR L1
KTSQDINKYMA

SEQ ID NO: 140 - protein, 7 aa, Artificial Sequence
  Natalizumab CDR L2
YTSALQP

SEQ ID NO: 141 - protein, 8 aa, Artificial Sequence
  Natalizumab CDR L3
LQYDNLWT

SEQ ID NO: 142 - protein, 12 aa, Artificial Sequence
  Palivizumab CDR H1
GFSLSTSGMSVG

SEQ ID NO: 143 - protein, 16 aa, Artificial Sequence
  Palivizumab CDR H2
DIWWDDKKDYNPSLKS

SEQ ID NO: 144 - protein, 10 aa, Artificial Sequence
  Palivizumab CDR H3
SMITNWYFDV

SEQ ID NO: 145 - protein, 10 aa, Artificial Sequence
  Palivizumab CDR L1
KCQLSVGYMH

SEQ ID NO: 146 - protein, 7 aa, Artificial Sequence
  Palivizumab CDR L2
DTSKLAS

SEQ ID NO: 147 - protein, 9 aa, Artificial Sequence
  Palivizumab CDR L3
FQGSGYPFT

SEQ ID NO: 148 - protein, 10 aa, Artificial Sequence
  Ranibizumab CDR H1
GYDFTHYGMN

SEQ ID NO: 149 - protein, 17 aa, Artificial Sequence
  Ranibizumab CDR H2
WINTYTGEPTYAADFKR

SEQ ID NO: 150 - protein, 13 aa, Artificial Sequence
  Ranibizumab CDR H3
YPYYYGTSHWFDV

SEQ ID NO: 151 - protein, 11 aa, Artificial Sequence
  L1
SASQDISNYLN

SEQ ID NO: 152 - protein, 7 aa, Artificial Sequence
  Ranibizumab CDR L2
FTSSLHS

SEQ ID NO: 153 - protein, 9 aa, Artificial Sequence
  Ranibizumab CDR L3
QQYSTVPWT

SEQ ID NO: 154 - protein, 8 aa, Artificial Sequence
  HMW-MAA CDR H1
GFTFSNYW

SEQ ID NO: 155 - protein, 10 aa, Artificial Sequence
  HMW-MAA CDR H2
IRLKSNNFGR

SEQ ID NO: 156 - protein, 13 aa, Artificial Sequence
  HMW-MAA CDR H3
TSYGNYVGHYFDH

SEQ ID NO: 157 - protein, 6 aa, Artificial Sequence
  HMW-MAA CDR L1
QNVDTN

SEQ ID NO: 158 - protein, 3 aa, Artificial Sequence
  HMW-MAA CDR L2
SAS

SEQ ID NO: 159 - protein, 9 aa, Artificial Sequence
  HMW-MAA CDR L3
QQYNSYPLT

SEQ ID NO: 160 - protein, 214 aa, Artificial Sequence
  WT IgE_VL
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 123 aa, Artificial Sequence
  HMW-MAA Variable Domain (Heavy Chain)
EQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 162 - protein, 107 aa, Artificial Sequence
  HMW-MAA Variable Domain (Light Chain)
DIELTQSPKFMSTSVCDRVSVTCKASQNVDTNVAWYQQKPGQSPEPLLFSASYRYTGVPDRFTGSGSGTDFTLTISNVQSEDLAEYFCQQYNSYPLTFGGGTKLEIK

SEQ ID NO: 163 - protein, 782 aa, Artificial Sequence; identical to SEQ ID NO: 26
  Trastuzumab IgE-IgG-Fc Heavy Chain

SEQ ID NO: 164 - protein, 782 aa, Artificial Sequence
  Trastuzumab IgE-IgG-Fc C220S Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 782 aa, Artificial Sequence
  Trastuzumab IgG-IgG-Fc dFcRn Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 166 - protein, 782 aa, Artificial Sequence
  Trastuzumab IgG-IgG-Fc dFcRn C220S Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 167 - protein, 214 aa, Artificial Sequence; identical to SEQ ID NO: 160
  Kappa Trastuzumab Light Chain

SEQ ID NO: 168 - protein, 550 aa, Artificial Sequence
  HMW-MAA IgE Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 169 - protein, 784 aa, Artificial Sequence
  HMW-MAA IgE IgG-Fc Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 784 aa, Artificial Sequence
  HMW-MAA IgE-IgG-Fc C220S Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 784 aa, Artificial Sequence
  HMW-MAA IgG-IgG-Fc dFcRn Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 172 - protein, 784 aa, Artificial Sequence
  HMW-MAA IgG-IgG-Fc dFcRn C220S Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 173 - protein, 214 aa, Artificial Sequence
  HMW-MAA Kappa Light Chain
DIQLTQSPSFLSASVGDRVTITCKASQNVDTNVAWYQQKPGKAPKPLLFSASYRYTGVPSRFSGSGSGTDFTLTISSLQPEDFATYFCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 15 aa, Artificial Sequence
  Modified IgG hinge region
EPKSSDKTHTCPPCP

SEQ ID NO: 175 - protein, 110 aa, Artificial Sequence
  Modified IgG CH2 domain
APELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAK

SEQ ID NO: 176 - protein, 107 aa, Artificial Sequence
  Modified IgG CH3 domain
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 177 - protein, 122 aa, Artificial Sequence
  HMW-MAA Alternative Variable Domain (Heavy Chain)
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSS

SEQ ID NO: 178 - protein, 107 aa, Artificial Sequence
  HMW-MAA Alternative Variable Domain (Light Chain)
DIQLTQSPSFLSASVGDRVTITCKASQNVDTNVAWYQQKPGKAPKPLLFSASYRYTGVPSRFSGSGSGTDFTLTISSLQPEDFATYFCQQYNSYPLTFGGGTKVEIK

SEQ ID NO: 179 - protein, 548 aa, Artificial Sequence
  Trastuzumab IgE Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK